(19) United States
(12) Patent Application Publication
(10) Pub. No.: US 2017/0356016 A1
(43) Pub. Date: Dec. 14, 2017

(54) MODIFIED MICROORGANISMS AND METHODS FOR PRODUCTION OF USEFUL PRODUCTS

(71) Applicant: ZUVASYNTHA LIMITED

(72) Inventors: Michelle GRADLEY, Welwyn Garden City, Hertfordshire (GB); Alex PUDNEY, Welwyn Garden City, Hertfordshire (GB); Dana HELDT, Welwyn Garden City, Hertfordshire (GB)

(21) Appl. No.: 15/515,441

(22) PCT Filed: Sep. 30, 2015
(86) PCT No.: PCT/EP2015/072552

§ 371 Date: Mar. 29, 2017

(30) Foreign Application Priority Data
Sep. 30, 2014 (GB) 1417268.8

(51) Int. Cl. C12P 7/24; C12N 9/02; C12N 9/88; C12N 15/52; C12P 7/18

(52) U.S. Cl. CPC C12P 7/24; C12N 15/52; C12P 7/18; C12Y 401/02004; C12Y 401/01001; C12N 9/88; C12N 9/0008; C12Y 102/01003; C07K 2319/00

(57) ABSTRACT

Non-naturally occurring microbial organisms and related methods, processes and materials are for microbial organisms that include a genetic modification which enhances production of 3-hydroxybutanal or a downstream product of 3-hydroxybutanal such as 1,3-butanediol from endogenous central metabolic intermediates such as acetyl CoA or pyruvate which are converted to acetaldehyde. Two molecules of acetaldehyde are condensed to form the 3-hydroxybutanal using an aldolase capable of accepting acetaldehyde as both the acceptor and donor in an aldol condensation. The aldolase may be a deoxyribose phosphate aldolase type enzyme, and is typically introduced into the organisms. Energetically favorable pathways produce 3-hydroxybutanal or downstream products thereof.

### TECHNICAL FIELD

The current invention relates generally to microorganisms, and related materials and methods, which have been modified to enhance their ability to produce commodity chemicals, for example, 1,3-butanediol and derivatives thereof, which can be produced in the microorganisms via the intermediates acetaldehyde and 3-hydroxybutanal.

### BACKGROUND ART

1,3-butanediol (1,3-BDO) is a four carbon diol which has a number of uses, including in the food, chemical and manufacturing industries.

1,3-BDO has traditionally been produced from petroleum derived acetylene via its hydration. The resulting acetaldehyde is then converted to 3-hydroxybutanal which is subsequently reduced to form 1,3-BDO. In more recent years, acetylene has been replaced by the less expensive ethylene as a source of acetaldehyde. However, as crude oil has become relatively more expensive than natural gas, many ethylene cracking operations are using lighter natural gas feedstocks to earn higher margins, leading to significantly lower quantities of C4 chemicals and rising prices.

Increasing the flexibility of inexpensive and readily available feedstocks while minimizing the environmental impact of chemical production are two goals of a sustainable chemical industry. Feedstock flexibility relies on the introduction of methods that enable access and use of a wide range of materials as primary feedstocks for chemical manufacturing. The reliance on petroleum based feedstocks for either acetylene or ethylene warrants the development of renewable, or cheaper, or non-petroleum derived feedstock based routes to 1,3-butanediol, butadiene and other valuable chemicals such as methylethylketone.

Publications in which microorganisms have been modified in such a way as to affect 1,3-BDO accumulation include the following: US20130109064; US20120329113; EP2495305A1; U.S. Pat. No. 8,268,607; US20110201068; US20100330635 and WO2014036140.

Nevertheless it can be seen that developing microorganisms and methods of their use to ferment sustainable and/or cheaper than traditional petroleum based feedstocks to 1,3-butanediol and other important chemicals, would provide a contribution to the art.

### DISCLOSURE OF THE INVENTION

The present invention relates to the engineering of organisms to imbue or enhance the ability to convert the central metabolic intermediates acetyl CoA and pyruvate to the common pathway intermediate acetaldehyde, which is then subject to an enzymatically catalysed aldol condensation, ultimately yielding 1,3-butanediol or other products. More specifically, in modified organisms of the invention, acetaldehyde derived from acetyl CoA or pyruvate as the primary pathway product is supplied as the substrate for an aldolase capable of the condensation of two molecules of acetaldehyde to form 3-hydroxybutanal.

The 3-hydroxybutanal, which is the product of this aldol condensation, can be directed to other products or other intermediates which can then in turn enter other natural or unnatural metabolic pathways.

Example intermediates include 2-hydroxyisobutyryl CoA, 3-hydroxybutyryl CoA, Crotonyl CoA, Crotonaldehyde, Butyryl CoA, Butanal, Acetoacetyl CoA, and acetoacetate.

Desirable downstream products include 2-hydroxisobutyrate, Crotyl alcohol, Crotonic acid, Butanol, Butyrate, 3-hydroxybutyrate, 1,3-butanediol, 3-hydroxybutylamine, Polyhydroxybutyrate, Acetone, and Isopropanol.

These products can, where desired, be recovered and used to make yet further commodities—for example butadiene, methacrylic acid, 2-methyl-1,4-butanediol, methyltetrahydrofuran, isoprene.

Any of these intermediate products, downstream products, and commodities may be referred to herein as “downstream products” or “products” herein for brevity.

A preferred product is 1,3-butanediol (1,3-BDO).

The modified organisms of the invention are typically microorganisms capable of using renewable or inexpensive feedstocks or energy sources such as sunlight, carbohydrates, methanol, synthesis gas (syngas) and\or other gaseous carbon sources such as methane to generate the appropriate metabolic intermediates.

As explained in more detail hereinafter, imbuing or enhancing the production of acetaldehyde from the central metabolic intermediate, or increasing its availability to the aldolase, will typically involve one or more of:

(i) introducing a heterologous gene encoding an enzyme having an activity utilised in generation of acetaldehyde from one or more of the central metabolic intermediates;
(ii) up-regulating at least one endogenous enzyme having an activity utilised in generation of acetaldehyde from one or more of the central metabolic intermediates; and/or
(iii) down-regulating or inactivating an endogenous enzyme which utilises acetaldehyde as a substrate (thereby making the acetaldehyde more available to the aldolase).

Typically, the aldolase capable of the in vivo condensation of two molecules of acetaldehyde to 3-hydroxybutanal will itself also be the product of genetic engineering e.g. via the introduction of a heterologous aldolase as described below.

A genetic modification combining these changes thus serves to increase the flux of central metabolic intermediates to the 3-hydroxybutanal via the acetaldehyde intermediate. In preferred embodiments this 3-hydroxybutanal is subsequently directed to a downstream product as described below.

Thus in one aspect there is provided a non-naturally occurring microbial organism which includes a genetic modification in its genome which enhances production of 3-hydroxybutanal by the microbial organism from at least one endogenous central metabolic intermediate via a 3-hydroxybutanal synthetic pathway in which two molecules of acetaldehyde are condensed to form 3-hydroxybutanal using an aldolase capable of accepting an aldehyde as both the acceptor and donor in an aldol condensation.

As will be well understood by those skilled in the art, “enhanced production” or production of an “increased amount” in the context of an intermediate product of a pathway should not be taken as requiring an increase in the absolute concentration, or steady stage concentration, of a product, in the microbial cell, although that may well result from increased production. Rather it will be understood to include a faster production of the product in question (i.e. a higher pathway flux through it) even where the product does not accumulate, but is subsequently converted to a further product.

Preferred organisms are those in which the modification enhances production of 1,3-butanediol (1,3-BDO) via a 1,3-BDO synthetic pathway in which the 3-hydroxybutanal is reduced to 1,3-BDO. In the disclosure below, therefore, particular emphasis is given to this embodiment. However it will also be understood that the invention applies likewise to modifications and pathways in which 3-hydroxybutanal is converted to other downstream products and, unless context demands otherwise, each of the embodiments relating to 1,3-BDO will be understood to apply mutatis mutandis to these other products. The genetic modification will be such that said modified organism produces a greater flux of or through 3-hydroxybutanal (and hence also of or through a downstream intermediate) to a product thereof such as 1,3-BDO) compared to a corresponding reference microbial organism not including said genetic modification, when grown on the same feedstock or energy source under the same conditions. For example the modified organism may produce at least 2, 3, 4, 5, 6, 7, 8, 9, 10, 20, 30, 40, 50, 60, 70, 80, 90, 100, 150, 200 times as much 3-hydroxybutanal or, more preferably, downstream product such as 1,3-BDO compared to the reference organism under the same conditions.

“Non-naturally occurring” in the present disclosure denotes the fact that the relevant modification which increases the flux to 3-hydroxybutanal or downstream product such as 1,3-BDO is introduced to a reference organism by human intervention.

As explained above, a microbial organism of the invention preferably includes one or more of the following modifications within its genome:

(i) a modification which increases the flux of a feedstock described herein to acetaldehyde, or makes acetaldehyde more available to the aldolase,
(ii) a modification which increases the flux of a feedstock described herein to 3-hydroxybutanal or a downstream product thereof.

For example the invention embraces a modification which confers on the microorganism the capability to convert a feedstock described herein to 1,3-BDO, wherein the microorganism lacks the ability to carry out that conversion in the absence of said modification. It also embraces a modification which increases the flux of a feedstock described herein to 1,3-BDO, in a microorganism where that flux is initially very low or negligible.

The modification typically relates to an aldolase enzyme as described herein, as well as a pathway providing acetaldehyde to it.

Preferably said enzyme is deoxyribose phosphate aldolase, EC 4.1.2.4 (“DERA”) or a variant thereof, or other enzyme sharing the ability to accept an aldehyde (acetaldehyde) as both the acceptor and donor in an aldol condensation

It will be appreciated that “genetic modification” can include more than one modification of the genome of the microbial organism in question.

Microbial organisms of the present invention may include any of the following genetic modifications in respect of the aldolase:

(i) introduction of at least one heterologous gene encoding the enzyme;
(ii) up-regulation of at least one endogenous gene encoding the enzyme.

A preferred embodiment is a microbial organism wherein said modification is introduction of a heterologous nucleic acid encoding the enzyme.

Optionally the heterologous gene encoding the enzyme may encode a fusion protein encoding also one or more other enzymes present in a 3-hydroxybutanal pathway—for example those involved in the provision of the aldolase substrate acetaldehyde. Optionally the heterologous gene encoding the enzyme may encode a fusion protein encoding also one or more other enzymes present in a downstream product pathway—for example those involved in the conversion of 3-hydroxybutanal to another product or intermediate.

The term “3-hydroxybutanal pathway” or “3-hydroxybutanal synthetic pathway” in the present context refers to a series of enzymatically catalysed reactions occurring in a cell which convert one or more principle chemical starting materials or substrates (feedstocks) to central metabolic intermediates comprising one or more of: pyruvate or acetyl CoA which are in turn converted to the common pathway intermediate acetaldehyde which is condensed to form 3-hydroxybutanal. A “3-hydroxybutanal (synthetic) pathway” may also include an activity involved in the conversion of 3-hydroxybutanal directly or indirectly to a downstream product derived from 3-hydroxybutanal, such as 1,3-BDO.

The term “3-hydroxybutanal pathway” enzyme should be construed accordingly i.e. an enzyme providing an activity in a “3-hydroxybutanal pathway”.

Thus an example of a “3-hydroxybutanal pathway” is a “1,3-BDO pathway” in which a series of enzymatically catalysed reactions occurring in a cell which convert one or more principle chemical starting materials or substrates (feedstocks) to 1,3-BDO via central metabolic intermediates comprising one or more of: pyruvate or acetyl CoA which are in turn converted to the common pathway intermediate acetaldehyde which is condensed to form the 1,3-BDO precursor, 3-hydroxybutanal, which is in turn converted to 1,3-BDO.

The conversion of central metabolic intermediates to the common pathway intermediate acetaldehyde may require 1 or more steps (e.g. 2, 3, 4 steps).

The invention embraces the introduction of all enzymes relevant to the 3-hydroxybutanal (e.g. 1,3-BDO) pathway, including those relating to early substrate utilisation and generation of the central metabolic intermediates themselves, as well as those involved in conversion of the central metabolic intermediates to the common intermediate.

Thus in addition to a modification relating to an aldolase, microbial organisms may include one or more other modifications within its genome:

For example said microbial organism may comprise two, three, four, five, six, seven, eight, nine, ten or more exogenous nucleic acids, each encoding a 3-hydroxybutanal (e.g, 1,3-BDO) pathway enzyme.

As explained in more detail below, the invention also embraces the knockout or other impairment of enzyme activities which would otherwise direct flux away from the pathway of choice e.g. direct flux of acetaldehyde away from the aldolase.

In one aspect the invention provides, inter alia, a non-naturally occurring microorganism that through genetic engineering gains the ability to produce 1,3-BDO or other downstream product derived from 3-hydroxybutanal from acetyl-coA, or gains the ability to produce an increased flux of 1,3-BDO or other downstream product derived from 3-hydroxybutanal from acetyl-coA, such that the 1,3-BDO or other downstream product accumulates and can be recovered or further converted enzymatically or chemically without recovery

As explained in more detail hereinafter, acetyl CoA may optionally be utilised via acetate. By way of non-limiting example, acetate can then be converted to acetaldehyde via carboxylic acid reductase activity, for example, EC 1.2.7.5 or EC. 1.2.99.6, ATP or ferredoxin driven or EC 1.2.1.30 or EC 1.2.1.3. Or can be converted to acetyl CoA via EC 6.2.1.1 or EC 2.8.3.8 and subsequently converted to acetaldehyde via EC 1.2.1.10.

Alternatively acetyl CoA may be utilised via pyruvate (see below, using enzymes such as EC 1.2.7.1 and EC 4.1.1.1) or via direct synthesis of acetaldehyde from acetyl CoA using an aldehyde dehydrogenase (acylating), for example, acetaldehyde dehydrogenase EC 1.2.1.10.

In another aspect the invention provides, inter alia, a non-naturally occurring microorganism that through genetic engineering gains the ability to produce 1,3-BDO or other downstream product derived from 3-hydroxybutanal from pyruvate, or gains the ability to produce an increased flux of 1,3-BDO or other downstream product derived from 3-hydroxybutanal from pyruvate, such that the 1,3-BDO or other downstream product derived from 3-hydroxybutanal accumulates and can be recovered or further converted enzymatically or chemically without recovery.

As explained in more detail hereinafter, pyruvate can be converted to acetaldehyde via acetyl CoA using enzymes such as EC 1.2.7.1 or EC 1.2.1.51 or EC 1.2.4.1 and EC 1.2.1.10. Alternatively, pyruvate can be converted to acetaldehyde, directly via pyruvate decarboxylase (EC 4.1.1.1).

It will be appreciated that although “pyruvate” may be referred to herein, depending on the pH and other conditions, it may likewise be present as pyruvic acid, and therefore all these descriptors are used interchangeably, unless context demands otherwise. This applies mutatis mutandis to other salts or acids described herein—e.g. acetic acid etc.

Also provided is a process or method for producing a microbial organism according to the invention, which comprises making a genetic modification as described herein.

The invention further provides a method for increasing the flux of 1,3-BDO or other downstream product derived from 3-hydroxybutanal produced by a microbial organism, which method comprises introducing one or more of the genetic modifications described herein into its genome.

Thus in some embodiments the present invention relates, amongst other things, to the generation of microorganisms that are effective at producing 1,3-butanediol from alternative substrates to traditional petroleum-based products.

Methods of producing such a microorganism will typically comprise the step expressing, or causing or allowing the expression of, a heterologous nucleic acid (for example, encoding at least an aldolase as described herein) within the host, following an earlier step of introducing the nucleic acid into the host or an ancestor of either. Suitable heterologous nucleic acids are discussed hereinafter. In other embodiments the methods may include the step of up-regulating native enzymes using genetic engineering and\or repressing enzymes to reduce flux to competing pathways.

Since the central intermediates acetyl CoA and pyruvate are present in all microbial systems, the actual choice of microbe utilised in the present invention will generally be based on the choice of feedstock or energy source which it is desired to use, along with the amenability of the microbe to genetic modification or introduction of a 1,3-BDO (or other downstream product derived from 3-hydroxybutanal) pathway. Preferred processes disclosed herein involve sustainable manufacturing practices that utilise renewable feedstocks, though other feedstocks which may provide cost or environmental benefits compared to traditional petroleum products may also be used, for example natural gas derived methanol.

For example the processes disclosed herein may utilise feedstocks such as syngas, CO₂, CO, and H₂, methane and methanol (shale gas or biomass/waste derived) to reduce energy intensity and cost and lower greenhouse gas emissions. Other feedstocks are discussed elsewhere herein.

Syngas is a mixture of primarily H₂ and CO that can be obtained via gasification of any organic feedstock, such as coal, coal oil, natural gas, biomass, or waste organic matter.

It will be appreciated that—unless context demands otherwise—where the term “syngas” is used, the embodiments of the invention will apply mutatis mutandis to other mixtures of carbon dioxide, carbon monoxide and/or hydrogen, and other substrates such as methane and methanol.

Thus the present invention preferably utilises microorganisms capable of utilizing syngas or other gaseous carbon sources (CO₂, CO) with or without methanol, methane or sugar co-utilisation or by use of methanol, methane or sugars directly as sole feedstocks. Or waste streams containing acetate. Photosynthetic organisms (e.g. algae) capable of using sunlight as an energy source are also expressly included.

In another aspect of the invention there is disclosed a method for producing 1,3-BDO or other downstream product derived from 3-hydroxybutanal that includes culturing the aforementioned non-naturally occurring microbial organisms under conditions and for a sufficient period of time to produce 1,3-BDO or other downstream product derived from 3-hydroxybutanal.

The term “cultured” or “culturing” on a feedstock as used herein is being used in a general sense to mean that the microbial organism utilises the feedstock in question for the production of the relevant product, and should not be taken to imply that the biomass of the microbial organism actually increases during the process.

In another aspect of the invention there is provided a process for producing 1,3-BDO or other downstream product derived from 3-hydroxybutanal, which process comprises culturing a microbial organism of the invention on a reaction feedstock as described herein so that it metabolises the feedstock to produce 1,3-BDO or other downstream product derived from 3-hydroxybutanal from central metabolic intermediates.

In some embodiments the microbe may be cultured in the presence of an additional energy source e.g. a carbohydrate such as a hexose, or sunlight.

The processes of the invention may further comprise recovering some or all of the 1,3-BDO or other downstream product derived from 3-hydroxybutanal e.g. by one or more of electrodialysis, solvent extraction, distillation, or evaporation. However more preferably 1,3-BDO or other downstream product derived from 3-hydroxybutanal may be converted chemically or enzymatically in situ to a downstream product or products, which may in turn be recovered by similar means.

The processes of the invention may further comprise converting the 1,3-BDO or other downstream product derived from 3-hydroxybutanal into a pharmaceutical, cosmetic, food, feed or chemical product, which may optionally be an unsaturated alcohol, alkene, carboxylic acid, ether, ester, or ketone e.g. methylethyl ketone, 1-butanol, 2-butanol, butadiene, isoprene and so on.

Thus in various aspects the invention provides non-naturally occurring microorganisms comprising one or more heterologous proteins conferring to the microorganism the capability to convert central intermediates to 1,3-BDO or other downstream product derived from 3-hydroxybutanal as described herein. Alternatively the heterologous protein may be directed at increasing the flux of reaction feedstocks such as syngas or other substrates described herein to 1,3-BDO or other downstream product derived from 3-hydroxybutanal, in a microorganism where that flux is initially very low or negligible under relevant industrial culture conditions.

In various aspects the invention provides a non-naturally occurring microorganism which has been modified to up-regulate (increase expression of) a native protein, or to modify the localisation of a native protein, or to modify the activity or specificity of a native protein, thereby conferring to the microorganism the capability to convert syngas or other substrates described herein to 1,3-BDO or other downstream product derived from 3-hydroxybutanal, wherein the microorganism lacks the ability to carry out that conversion in the absence of said modification. Alternatively the heterologous protein may be directed at increasing the flux of metabolic intermediates from the feedstock being utilised in a microorganism where that flux is initially very low or negligible.

Thus the invention provides a non-naturally occurring microbial organism having a genetically modified 3-hydroxybutanal or 1,3-BDO biosynthetic pathway and the competence to metabolise syngas or other feedstocks or energy source described herein to produce 1,3-BDO or other downstream product derived from 3-hydroxybutanal.

Some aspects and embodiments of the invention will now be discussed in more detail:

### Metabolic Pathways Leading to Central Metabolites, and Increased Production or Availability of Acetaldehyde

Pyruvate and acetyl CoA are products of a considerable range of different central metabolic pathways for assimilation of carbon. They are converted to important cellular building blocks essential for life. In the present invention they are utilised within a 1,3-BDO biosynthetic pathway, which pathway is at least in part the result of genetic engineering of the microbial organism.

As explained above, typically an organism is selected according to the feedstock it is desired to utilise. The organism may be selected to have in its genome a particular metabolic pathway leading to acetyl CoA and\or pyruvate. Example metabolic pathways include:
the Wood-Ljungdahl pathway (FIG. 5)
the ribulose monophosphate (RuMP) pathway (FIG. 4)
the reverse TCA cycle (FIG. 6)
the serine cycle (FIG. 7)
glycolysis and the pentose phosphate pathway
the Calvin cycle via 3-phosphoglycerate (FIG. 9)
other pathways such as the 3-hydroxypropionate/4-hydroxybutyrate cycle

Pathways generating these central metabolic intermediates are well reported and well understood by those skilled in the art. The Wood-Ljungdahl pathway, reverse TCA cycle, the serine cycle, the RuMP pathway and the Calvin cycle, are examples of C1 (gas and liquid) fixation pathways. In some case these pathways can be used alongside glycolysis.

For example, the Wood-Ljungdahl pathway is important for redox balancing by using the reducing equivalents generated from glycolysis and pyruvate decarboxylation to acetyl CoA, to fix the released 2 CO₂ into a further molecule of acetyl CoA.

The serine or the RuMP pathways are generally used by methanotropic and methylotrophic organisms for assimilation of C1 feedstocks such as methanol, methane and CO₂. These pathways are well described and well known in the art. The product of the RuMP pathway is pyruvate which would normally be converted primarily to biomass.

Intercepting pyruvate via, for example, decarboxylation to acetaldehyde would redirect flux towards 1,3-butanediol synthesis using the described invention. The Calvin cycle is used by photosynthetic organisms such as algae for assimilation of CO₂ using light energy.

The serine cycle primarily produces acetyl CoA which normally enters the ethylmalony CoA pathway for synthesis of C4 building blocks for biomass synthesis. If acetyl-CoA is required as the biosynthetic precursor of membrane fatty acids or the storage compound poly 3-hydroxybutyrate for example, then the EMC pathway is not required for oxidation of acetyl-CoA, (Anthony, C. 2011. Science Progress, 94, 109). Hence, acetyl CoA can be tapped off to other more useful compounds such as 1,3-butanediol via conversion of acetyl CoA to acetaldehyde using a pathway described herein.

The same principle can be used for all metabolic pathways producing pyruvate or acetyl CoA as the product. Another example would be the reverse TCA cycle which again produces acetyl CoA from fixation of two molecules of CO₂. All these central metabolic pathways producing acetyl CoA or pyruvate are well understood in the art.

A diverse range of microorganisms with known pathways capable of utilising syngas, or gases such as CO, CO₂ and H₂.

Microorganisms which are CO utilisers are termed “carboxydotrophic microorganisms”. Such organisms can be aerobes and anaerobes.

Anaerobic examples of these microorganisms fall into 3 main groups: those producing mainly acids (e.g. acetic acid, termed “acetogens”), those producing mainly methane and those producing mainly hydrogen. The first group is of particular interest in the present invention:

Carboxydotrophic acetogens are acetogenic microorganisms capable of utilising the syngas components CO and H₂ via the Wood-Ljungdahl pathway (FIG. 2) producing the key intermediate acetyl CoA.

The Wood-Ljungdahl pathway is well known in the art (see FIG. 5) and can be separated into two branches: the methyl branch (reductive branch) and the carbonyl branch. The methyl branch converts syngas (CO or CO₂) to methyl-tetrahydrofolate (methyl-THF) whereas the carbonyl branch supplies a molecule of CO which along with methyl-THF is converted to acetyl-CoA.

“Acetogens” as used herein refers to anaerobic organisms able to reduce CO₂/CO to acetate via this pathway. Acetogens can grow on a variety of different substrates such as, for example, hexoses [glucose, fructose and xylose], C2 and C1 compounds [gas and liquid] including methanol (see FIG. 2), CO₂/H₂ and CO gases. Acetogens are also known to utilise acetate directly.

Over one hundred acetogenic species, representing twenty-two genera, have been isolated so far from various habitats such as sediments, sludge, soils and the intestinal tracts of many animals, including termites and humans. From the twenty-two genera, Clostridium and Acetobacterium harbour the most known acetogenic species (Drake et al., Ann. N. Y. Acad. Sci 1125:100-108 (2008)). However it should be noted that the Wood-Ljungdahl pathway is actually not restricted to acetogens and is present in many anaerobic bacteria as a means of fixing CO₂.

While defined as strict anaerobes some acetogens have been isolated from different aerobic or microaerobic environments (Annals New York Academy of Sci. 2008, 1125, 100). It has been demonstrated that these types of bacteria are equipped with an assortment of oxidative stress enzymes and that certain acetogens can even reduce oxygen by different means (Tirado-Acevedo, O. PhD thesis 2010). Clostridium ljungdahlii has been shown to tolerate up to 8% oxygen in the gas phase (ibid).

Acetogens are becoming a significant focus for the biotech industry, as important bulk chemicals can potentially be produced from autotrophic growth at the expense of CO₂ via syngas fermentation or via coupling with methanol, which can serve as a source of carbon and energy in the absence of syngas. in the presence of a more oxidised substrate such as CO₂.

Acetogens can utilise hexoses (e.g. glucose, fructose and xylose) and other sugars as substrates.

In many acetogens acetate is the primary product of hexose consumption:

1C₆H₁₂O₆→3CH₃COOH

The pathway of hexose consumption starts with their oxidation via the Embden-Meyerhof-Parnas pathway to pyruvate, which is then oxidized by pyruvate:ferredoxin oxidoreductase to acetyl-CoA, reduced ferredoxin, and CO₂. The acetyl-CoA is then converted to acetate via acetyl phosphate.

This oxidative branch of the pathway to pyruvate is coupled to the synthesis of 4 mol of ATP by SLP:

C₆H₁₂O₆+4ADP+4Pi→2CH₃COOH+2CO₂₊₄ATP+8 [H]

Second, the reducing equivalents 8[H], gained during glycolysis and pyruvate:ferredoxin-oxidoreductase are reoxidized by reducing the two mol of CO₂ to another mol of acetate via the Wood-Ljungdahl pathway

2 CO₂+8 [H]+nADP+nPi→CH₃COOH+nATP. The exact amount of net ATP generated other than from acetyl CoA to acetate via SLP, is not yet known.

During growth on sugars or other organic substrates, CO₂ can be formally regarded as an electron sink and per se, there is no need that the Wood-Ljungdahl pathway is coupled to energy conservation. Energy is gained during glycolysis, and the redox balance is maintained by operation of the Wood-Ljungdahl pathway (FIG. 2). However, it is important to note that the Wood-Ljungdahl pathway also enables growth on CO₂ and hydrogen or carbon monoxide or on methanol and CO₂ or formate. Particularly in the case of the gaseous substrates it must be coupled to net ATP synthesis. The overall free energy change of the reaction (ΔGₒ′=−95 kJ/mol) could allow for the synthesis of 1 to 2 mol of ATP.

The energetics of this pathway are finely balanced. One mole of ATP is produced by SLP in the acetate kinase reaction via acetyl phosphate from acetyl CoA, but one mole of ATP is consumed in the formyl-H₄F synthetase reaction. Therefore, the net ATP gain by SLP is zero, and ion gradient-driven phosphorylation is known to occur as well contributing to the generation of more ATP.

The above discussion clearly highlights under most circumstances the important role of SLP level phosphorylation ATP synthesis via conversion of acetyl CoA to acetate, to balance the Wood-Ljungdahl pathway and indeed considerable flux to acetate is seen in growing acetogens. Driving flux away from acetate synthesis to any more useful chemical in high yield, impacts to varying degrees on an acetogen's energy balance as acetate synthesis is a key ATP synthesis step. Even when growing on methanol and CO₂ where the energetics may be more favourable, in many cases preventing synthesis of acetate may still have some impact. Further, the metabolism of many acetogens has evolved primarily for the efficient synthesis of either biomass or acetate from the central intermediate acetyl CoA. Hence, industrial products derived directly from acetyl CoA or acetate may be preferred for process development using acetogens.

Employing an aldolase capable of accepting the product acetaldehyde as a substrate (both as acceptor and donor) permits the efficient generation of useful chemical products from acetate which acetogens naturally accumulate in high yield and high concentration.

Routes for utilising acetyl-CoA derived acetate for generation of acetaldehyde are explained in more detail below. A preferred route of acetaldehyde generation would be based on utilisation of ferredoxin driven aldehyde ferredoxin oxidoreductase. This enzymatic conversion does not require ATP so may be particularly important for bacteria growing on C1 gases such as CO, CO₂/H₂ or syngas for the energetics reason described above. Since the natural SLP step involving the conversion of acetyl CoA to acetate can be conserved, the energetics of the Wood-Ljungdahl pathway would be unchanged.

However other routes for acetate reduction, including those which require ATP, may be utilised where the energetics of the system permit. Overall, the ability to create a C4 compound such as 3-hydroxybutanal from a C2 compound such as acetaldehyde without the normal requirement for energy, is highly desirable when efficiently exploiting microbial metabolism for industrial chemical synthesis.

As noted above, organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved.

Thus in one embodiment the invention provides a non-naturally occurring microorganism having the Wood-Ljungdahl pathway and the capability of utilising syngas naturally and that through genetic engineering gains the ability to produce 1,3-BDO or gains the ability to produce an increased flux of 1,3-BDO.

Many acetogens have been shown to grow in the presence methanol if another more oxidised co substrate such as CO₂, is present. The provision of reducing equivalents is crucial to drive this process to either natural products such as acetate, or unnatural acetogen products such as 1,3-butanediol.

Hydrogen is a major source of reducing equivalents, but equally for example, dissimilation of methanol can also generate reducing equivalents (6[H]) and ATP energy. Further, as shown in FIG. 2, methanol utilisation confers an energetic advantage because it provides a preformed methyl group for synthesis of acetyl CoA eliminating the need for an ATP for conversion of formate to formyl-THF catalysed by formyl-THF synthetase (Bainotti, A. E and Nishio, N. 2000. J. Appl. Microbiol, 88, 191). The specific energetic requirements of the transfer of the methyl group from methanol is not clearly understood. However, catalytic amounts of ATP are cited in the literature and Stupperich, E and Konle, R, 1993, Appl. Environ. Microbiol. 59, 3110, describe a requirement of approximately 0.3 ATP per methyl group transfer. Hence, a saving of approximately 0.7ATP may be achieved when a preformed methyl group is provided instead of CO₂. If only a catalytic amount of ATP is required, the energy benefit may be greater. However, as highlighted, a clear understanding of methanol utilising energetics is not yet established in the art.

In the case of CO₂, additional sources include, but are not limited to, production of CO₂ as a byproduct in ammonia and hydrogen plants, where methane is converted to CO₂; combustion of wood and fossil fuels; production of CO₂ as a byproduct of fermentation of sugar in the brewing of beer, whisky and other alcoholic beverages, or other fermentative processes; thermal decomposition of limestone, CaCO₃, in the manufacture of lime, CaO; production of CO₂ as byproduct of sodium phosphate manufacture; and directly from natural carbon dioxide springs, where it is produced by the action of acidified water on limestone or dolomite.

The ability of acetogens to utilise methanol requires specific methyltransferases. Where such aceteogen methyltransferases are not naturally present, an acetogen can be engineered with heterologous methyltransferases and other associated proteins to allow it to utilise methanol as well as the other feedstocks discussed above.

Examples of enzymes required to give an acetogen the ability to grow on methanol include:

methanol methyltransferase (MtaB)
Corrinoid protein (MtaC),
Methyltetrahydrofolate:corrinoid protein methyltransferase (MtaA),
Methyltetrahydrofolate:corrinoid protein methyltransferase (AcsE),
Corrinoid iron-sulfur protein (AcsD)

The methylotrophs and methanotrophs also naturally grow on methanol and/or methane, utilising for example, the RuMP (FIG. 4) or serine cycle (FIG. 7) pathways for C1 metabolism. As for the Wood-Ljungdahl pathway, both the serine cycle of C1 metabolism and the RuMP pathway are well described and well understood in the art.

Thus in one embodiment the invention provides a non-naturally occurring microorganism having the RUMP or serine cycle pathway encoded in its genome and the capability of utilising methanol or methane naturally and that through genetic engineering gains the ability to produce 1,3-BDO or gains the ability to produce an increased flux of 1,3-BDO.

Photosynthetic organisms such as microalgae or cyanobacteria are autotrophs or heterotrophs able to utilise sunlight (light energy) for CO₂ fixation via the Calvin cycle. A product is glyceraldehyde-3-phosphate which can be converted to sugar or to pyruvate and acetyl CoA.

Many diverse microorganisms are heterotrophic and can utilise sugars as a source of carbon and energy via glycolytic pathways such as the Entner doudoroff pathway, Embden meyerhof pathway or pentose phosphate pathway. All sugar assimilation pathways are well understood in the art. A product of these pathways is pyruvate which may be converted to acetyl CoA for example, for entry into the TCA cycle for supply of cellular building blocks such as malate, oxaloacetate, succinate or fumarate.

As discussed above in relation to acetogens, many organisms not normally considered heterotrophs (e.g. acetogens or methylotrophs) are also capable of heterotrophic growth if sugars are supplied.

Photoheterotrophs are heterotrophic phototrophs—that is, they are organisms that use light for energy, but cannot use carbon dioxide as their sole carbon source, instead using carbohydrates, fatty acids, and alcohols and so on. Examples of photoheterotrophic organisms include purple non-sulfur bacteria, green non-sulfur bacteria, and heliobacteria. Thus feedstocks when utilised in the present invention may where desired may include or indeed consist of sugars as all or part of the source of carbon and\or energy

Enzymes suitable for converting the metabolic intermediates to acetaldehyde are discussed in more detail in the Examples below, and in FIG. 3. Briefly

Route 1 proceeds from acetyl CoA through acetate, (a natural product of acetogenic microorganisms), to acetaldehyde via carboxylic acid reductase activity (Activity A), using for example, EC 1.2.7.5 or EC. 1.2.99.6, ATP or ferredoxin driven or EC 1.2.1.30 or EC 1.2.1.3.

Route 2 involves direct synthesis of acetaldehyde from acetyl CoA (Activity B) using an aldehyde dehydrogenase (acylating), for example, acetaldehyde dehydrogenase EC 1.2.1.10.

Route 3 involves the conversion of pyruvate to acetaldehyde via acetyl CoA (Activity C and B) using enzymes such as EC 1.2.7.1 or EC 1.2.1.51 or EC 1.2.4.1 and EC 1.2.1.10.

Route 4 involves the conversion of pyruvate to acetaldehyde, (Activity D) directly via for example, pyruvate decarboxylase (EC 4.1.1.1).

Route 5 involves the conversion of acetyl CoA to acetaldehyde via pyruvate (Activity E and D) using enzymes such as EC 1.2.7.1 and EC 4.1.1.1.

Route 6 involves the conversion of acetate to acetaldehyde via acetyl CoA (Activity F and B) using enzymes such as EC 6.2.1.1 or EC 2.8.3.8 and EC 1.2.1.10.

It will be appreciated that in any given organism of the invention, more than one of these routes may be utilised.

The enzyme aldehyde ferredoxin oxidoreductase (EC 1.2.7.5) can be found in many acetogens and other organisms and has been shown to be capable of reducing unactivated carboxylic acids to the corresponding aldehyde (White, H et al. Biol. Chem Hoppe Seler 1991, 372 (11) 999; White, H and Simon, H. Arch. Microbiol, 1992, 158, 81; Fraisse. L and Simon, H. Arch. Microbiol. 1988, 150, 381; Basen et. al. 2014. PNAS, 111 (49), 17618). Kopke, M. et al. PNAS, 2010, 107, 15305, describes genes capable of reduction of acetate to acetaldehyde in the acetogen Clostridium ljungdahlii.

Alternative means of generating acetaldehyde in acetogens for supply to an aldolase which could be used separately or in conjunction with reduction of acetate are as follows:

Acetaldehyde dehydrogenase (EC 1.2.1.10) or any aldehyde dehydrogenase capable of converting acetyl CoA to acetaldehyde directly may be used. As explained above, in this case the SLP step from conversion of acetyl CoA to acetate would be lost. Some compensation for this loss could be achieved from ion gradient phosphorylation from the Wood-Ljungdahl pathway when growing on gases such as CO, CO₂/H₂ or syngas. ATP may also be synthesised via NAD(P) reduction coupled to reduced ferredoxin, but growth on methanol and CO₂ or another more oxidised co substrate may be most suited due to the potential more favourable energetics and potential for supply of reducing equivalents and ATP from methanol dissimilation. Another example of an alternative would involve a carboxylic acid reductase (CAR) enzyme (EC 1.2.99.6). These enzymes catalyse reduction of carboxylic acids to the corresponding aldehyde via activation with ATP. The energetics of this route would be similar to that described for acetaldehyde synthesis from acetyl CoA via acetaldehyde dehydrogenase. The use of a carboxylic acid reductase in a 1,3-butanediol pathway for synthesis of a corresponding aldehyde is described in U.S. Pat. No. 8,268,607, albeit that the pathway for synthesis is different.

The other enzymes utilised in these routes are discussed in more detail in Examples hereinafter.

### Down Regulation of Native Enzymes

The modified organisms of the invention may be engineered to target (down-regulate, knockout or inhibit) the activity of enzymes which may otherwise direct the flux of intermediates in the 3-hydroxybutanal pathway to other products or biomass. Methods of targeting genes in this way are known in the art, and also discussed below.

In acetogens, if the bioenergetics allow loss of ATP synthesis from acetyl CoA conversion to acetate, acetate accumulation can be reduced by targeting of phosphotransacetylase (pta) or acetate kinase (ack) genes. This can enhance the level of acetyl CoA, which can be utilised directly or via pyruvate. Thus where utilising Route 2, it may be desired to target EC 2.3.1.8 (phosphotransacetylase) or EC 2.7.2.1 (acetate kinase) or both.

Where utilising Route 3 or 4 or 5 or 6, it may be desired to target LDH activity (EC 1.1.1.27 or 1.1.1.37; the latter is malate dehydrogenase but is known to accept pyruvate as a substrate) to prevent loss of pyruvate to lactate, and \or (where appropriate) to target pyruvate formate lyase (EC 2.3.1.54). In all cases the purpose is to prevent or minimise loss of pyruvate to other products.

Alternatively or additionally it may be desired to increases the availability of acetaldehyde to the aldolase by down-regulation or inactivation of an endogenous enzyme (e.g. an alcohol dehydrogenase) which utilises acetaldehyde as a substrate for some other purpose e.g. production of ethanol. Increasing the availability to the aldolase of the acetaldehyde increases production of the 3-hydroxybutanal from the aldolase.

For way of further example, it may particularly be desired to target any alcohol dehydrogenase with a preference for reduction of acetaldehyde to ethanol relative to reduction of 3-hydroxybutanal to 1,3-BDO. These acetaldehyde to ethanol enzymes are generally classified in EC 1.1.1.1.

### Aldolase Enzymes

In the modified organisms of the invention, acetaldehyde derived from acetyl CoA or pyruvate is used to supply the substrate for a DERA type aldolase (deoxyribose phosphate aldolase, EC 4.1.2.4, DERA or DERA like′ enzyme) capable of the condensation of two molecules of acetaldehyde to form 3-hydroxybutanal (via “Reaction G” in FIG. 3). Example enzymes are given in Table 6.

The natural deoxyribose phosphate aldolase (DERA) reaction is:

2-deoxy-D-ribose 5-phosphate [symbol] D-glyceraldehyde 3-phosphate+acetaldehyde

The phosphorylated substrate is preferred but most wild type enzymes will catalyse the condensation of two non-phosphorylated aldehyde molecules. Primarily acetaldehyde and another aldehyde. An example of a DERA which accepts phosphorylated and non-phosphorylated substrates with approximately equal preference is described by Zhong-Yu, Y. et al. (J. Ind. Microbiol Biotech. 2013, 40, 29).

Evolution and development of DERA for synthesis of key pharmaceutical intermediates has received considerable focus over the past 25 years (DeSantis, G et al. Bioorg & Medicinal Chem. 2003, 11, 43). However, it has not previously been suggested to integrate a DERA type enzyme into an unnatural pathway for synthesis of 1,3-butanediol or other downstream products.

Under certain conditions many DERA enzymes are also capable of catalysing a tandem sequential condensation of three acetaldehydes, which will preferably be avoided in the present context. Fortunately the undesired sequential reaction involving two aldol condensations of acetaldehyde (FIG. 8) is generally not the dominant reaction for DERA catalysed aldol condensations. The desired monoaldol product (3-hydroxybutanal) accumulates and high levels of wild type DERA enzymes are required to drive the reaction to accept a second acetaldehyde addition (Green Chemistry in the Pharmaceutical industry, 2010, John Wiley and sons). Hence the interception of the monoaldol (3-hydroxybutanal) and direction to other products is feasible, for example, by reduction to 1,3-butanediol. Furthermore, DERA enzymes are known (and can be generated) which are very inefficient at catalysing the second aldol condensation, thereby producing just the monoaldol 3-hydroxybutanal (see e.g. U.S. Pat. No. 7,402,710) which describes a DERA enzyme from the organism Pyrobaculum aerophilum). U.S. Pat. No. 7,402,710 describes synthesis of C4 hydroxyaldehydes such as 3-hydroxybutanal but not in the context of a metabolic pathway or where acetaldehyde is supplied by an enzyme in the pathway. In U.S. Pat. No. 7,402,710, acetaldehyde is added exogenously to an isolated DERA enzyme preparation

Many DERAs are known to be inactivated at aldehyde concentrations above 100 mM and may be sensitive to concentrations below this concentration, for both acetaldehyde and 3-hydroxybutanal and this has been a limitation for application of DERA for synthesis of statin intermediates via sequential condensation of chloroacetaldehyde and two molecules of acetaldehyde (Green Chemistry in the Pharmaceutical industry, 2010, John Wiley and sons). In the context of the current invention DERA is used as part of an unnatural pathway for synthesis of 1,3-butanediol and other valuable chemicals where the substrate acetaldehyde is provided via de novo synthesis from a preceding pathway enzyme. Thus aldehyde concentrations in the processes of the invention will never approach 100 mM, and sensitivity to this concentration of acetaldehyde is therefore immaterial. Both acetaldehyde and 3-hydroxybutanal are intermediates in the pathway and accumulation of these intermediates will be avoided by ensuring adequate activity of pathway enzymes to maximise carbon flux to 1,3-butanediol or other target chemicals.

Wild type DERA aldolase has been overexpressed in E. coli and run as a high intensity process for synthesis of chiral lactol intermediates for the statin pharmaceuticals (Oslaj, M. et al Plos one, 8 (5), 1). The process involves a fed batch approach involving the condensation of a 2-substituted acetaldehyde and acetaldehyde to the corresponding lactols in a tandem sequential synthesis. Although this process was run as a whole cell system, the reactants were fed to the cells and were not generated in situ. Furthermore there were no modifications made which would have enhanced production or availability of endogenous acetaldehyde from central metabolic intermediates.

The processes of the present invention do not utilise batch feeding of the microbial organisms with 2-substituted acetaldehyde and/or acetaldehyde.

### Fusions

As explained above, the aldolase such as DERA may be provided as a fusion protein encoding also one or more other enzymes involved in the provision of the aldolase substrate acetaldehyde, or linked to such other enzymes using chemical or other means (e.g. scaffoldins or dockerins). Examples include fusions of DERA with an acetaldehyde dehydrogenase or pyruvate decarboxylase or a carboxylic acid reductase such as AOR which catalyse reactions B, D and A described herein (see Tables 2, 4, 1). Example 11 demonstrates the production of a DERA-EutE fusion.

In other embodiments, the aldolase such as DERA may be provided as a fusion protein encoding also one or more other enzymes involved in a downstream product pathway, or linked to such other enzymes using chemical or other menas (e.g. scaffoldins or dockerins). The enzyme may, for example, be one involved in the conversion of 3-hydroxybutanal to another product or intermediate. Examples include enzymes listed in Table 7.

### Production of 1-3 BDO and Downstream Products, and Utilities for 1,3-BDO

Reduction of the aldehyde moiety of the aldolase catalysed acetaldehyde condensation product 3-hydroxybutanal to the corresponding alcohol prevents further condensation of a third acetaldehyde. 3-Hydroxybutanal is reduced to (via “Reaction H” in FIG. 3) 1,3-butanediol by an alcohol dehydrogenase or aldehyde reductase, for example, using enzymes categorised in EC 1.1.1.-.

This reaction is preferably catalysed by a medium chain alcohol dehydrogenase or aldehyde reductase, ideally which shows preference for alcohols of C4 or greater, for example see Appl. Environ. Microbiol, 2000, 66, 5231. More specifically the enzyme preferably shows a preference for reduction of 3-hydroxybutanal to 1,3-BDO relative to reduction of acetaldehyde to ethanol (Example 9). An alcohol dehydrogenase described by Wales, M and Fewson, C. Microbiol 1994, 140, 173 again shows preference for longer chain alcohols. Although measured in the oxidative direction, the dehydrogenase also accepts 1,4-butanediol as a substrate. 2,3-butanediol is not a substrate, clearly demonstrating the desired primary alcohol as opposed to secondary alcohol specificity for application to 3-hydroxybutanal reduction. Other enzymes which it may be desired to utilise for conversion of 3-Hydroxybutanal to 1,3-butanediol are described in Example 3 and Table 7 hereinafter.

The use of a 3-hydroxybutanal reductase (alcohol dehydrogenase) within a 1,3-butanediol pathway is described in US20130109064 but there is no suggestion therein of the presently disclosed pathway for synthesis of 3-hydroxybutanal.

1,3-BDO has numerous utilities in industry. For example, 1,3-BDO is commonly used as an organic solvent for food flavoring agents. It is also used as a co-monomer for polyurethane and polyester resins and is widely employed as a hypoglycemic agent. Optically active 1,3-BDO is a useful starting material for the synthesis of biologically active compounds and liquid crystals. Another use of 1,3-butanediol is that its dehydration affords 1,3-butadiene and other important chemicals such as methylethyl ketone (Ichikawa et al., J. Molecular Catalysis A-Chemical, 231:181-189 (2005); Ichikawa et al., J. Molecular Catalysis A-Chemical, 256:106-112 (2006)), 1,3-butadiene is an important chemical used to manufacture synthetic rubbers (e.g. tyres), latex, and resins. 1,3-Butadiene and further examples of products produced by chemical conversion of 1,3-butanediol are shown in FIG. 1.

Production of Other Downstream Products from 3-Hydroxybutanal

It will be appreciated that the aldol condensation product 3-hydroxybutanal can also be directed to products other than 1,3-BDO. In that sense, 3-hydroxybutanal can be considered a branch point for a number of possible unnatural DERA-based pathways leading to a variety of immediate or further downstream products.

By way of non-limiting example, 3-Hydroxybutanal can be converted (e.g. oxidised, reduced) to:
3-hydroxybutyrate
1,3-butanediol
   which can in turn be utilised or recovered per se, or converted to other products via other natural or unnatural metabolic pathways. 3-hydroxybutyrate has utility as a biodegradable plastics monomer.

Further, 3-hydroxybutanal can be also be converted to metabolic intermediates such as 3-hydroxybutyryl CoA using for example butanal dehydrogenase (EC 1.2.1.57) or another aldehyde dehydrogenase such as EC1.2.1.10 which can allow metabolic access to a range of other products. Aldehyde dehydrogenases have been mutated to improve their preference for C4 aldehydes relative to C2 aldehydes (e.g. acetaldehyde). For example Baker et al. describe a mutant with a preference for butanal relative to acetaldehyde, Biochemistry. 2012 Jun. 5; 51(22):4558-67. Epub 2012 May 21. This enzyme may have utility in the conversion of 3-hydroxybutanal to 3-hydroxybutytyl CoA. Several other enzymes have a natural preference for a C4 aldehyde. Yan, R. T and Chen, J. S. 1990 Appl Environ Microbiol 56, (9) 2591. Any of these enzymes could, if desired, be further engineered to optimise their activity in generating 3-hydroxybutyryl CoA in the context of the present invention.

Downstream products from 3-hydroxybutyryl CoA include:
2-hydroxyisobutyric acid (an intermediate for methacrylic acid synthesis).
polyhydroxybutyrate (which also has utility in biodegradable plastics).
Crotyl alcohol (which can be converted enzymatically or chemically to 1,3-butadiene).

Butanol (which can be converted enzymatically or chemically to 1,3-butadiene) Other downstream products include Crotonic acid, butyrate, 3-hydroxybutyrate, 3-hydroxybutylamine, Polyhydroxybutyrate, Acetone, and isopropanol.

The synthesis of products from 3-hydroxybutyryl CoA is established biochemistry. For example as described in the following references. Toshiyuki, U. et al. 2014, mbio 5, (5), 1 (butyrate); Torben, H. et al. 2010, Appl. Microbiol Biotechnol. 88, 477 (2-hydroxyisobutyric acid); Nadya, Y. et. al. 2012. J. Biol. Chem. 287 (19) 15502 (2-hydroxyisobutyric acid); Rehm, B. H. 2007. Curr. Issues. Mol. Biol. 9 (1), 41 (polyhydroxybutyrate); Lee, S. Y et. al. 2008. Biotechnol Bioeng. 101 (2), 209 (butanol); U.S. Pat. No. 8,580,543; WO2013057194 (crotyl alcohol).

A route to 3-hydroxybutyryl CoA via acetate in acetogens allows for generation of this intermediate without sacrificing the ATP energy which would otherwise be lost if 3-hydroxybutyryl CoA was provided via, for example, acetyl CoA to acetoacetyl CoA. This is because preventing acetate formation from acetyl CoA loses the molecule of ATP generated from the acetate kinase reaction. Generation of acetoacetyl CoA is energetically unfavourable under most conditions. The pathway to 3-hydroxybutyryl CoA via acetate through the DERA pathway described herein, retains the energetics of acetogenesis. Hence, the same product is reached through a more energetically favourable route. The key intermediate branch point is the DERA product 3-hydroxybutanal.

### Genetic Modification and Hosts

Based on the guidance provided herein, those skilled in the art will understand that the number of encoding nucleic acids to introduce in an expressible form will reflect any 3-hydroxybutanal or downstream product derived therefrom (e.g. 1,3-BDO) pathway deficiencies of the selected microbial host. Therefore, a non-naturally occurring microorganism of the invention can have one, two, three, or more, up to all nucleic acids encoding the enzymes or proteins constituting a 3-hydroxybutanal or downstream product derived therefrom (e.g. 1,3-BDO) pathway revealed herein. In some examples, the non-naturally occurring microorganisms can also include other genetic modifications that facilitate or optimise 1,3-BDO (or other downstream product derived from 3-hydroxybutanal) biosynthesis or that confer other useful functions onto the host microorganism.

Preferred microbial organisms are discussed herein and include, inter alia, yeasts such as Saccharomyces cerevisiae, Kluveromyces lactis Candida boidinii, Pichia angusta, Ogataea polymorpha, Komagataella pastoris.

Other preferred microbial organisms include bacteria such as Moorella thermoacetica, Moorella thermoautotrophica, Thermoacetogenium phaeum, Thermoanaerobacter kivu, Acetobacterium woodii, Clostridium carboxidivorans, Clostridium drakei, Clostridium formicoaceticum, Clostridium glycolicum, Clostridium magnum, Clostridium mayombei, Clostridium ljungdahlii, Clostridium ragsdalei, Clostridium aceticum, Clostridium autoethanogenum, Clostridium scatologenes, Acetitomaculum ruminis, Acetogenium kivui, Eubacterium limosum, Oxobacter pfennigii, Acetobacterium tundrae, Acetobacterium noterae, Acetobacterium carbinolicum, Acetobacterium dehalogenans, Acetobacterium fimetarium, Acetobacterium malicum, Acetobacterium paludosum, Acetobacterium wieringae, Acetohalobium arabicum, Acetonema longum, Acetitomaculum ruminis, Acetoanaerobium noterae, Acetobacterium bakii, Butyribacterium metholytrophicum, Blautia hydrogenotrophica, Blautica coccoides, Blautia producta, Blautia schinkii, Peptostrepococcus productus, Sporomusa acidovorans, Sporomusa aerivorans, Sporomusa malonica, Sporomusa ovate, Sporomusa paucivorans, Sporomusa rhizae, Sporomusa silvacetica, Sporomusa spaeroides, Sporomusa termitida. Bacillus methanolicus, Methylobacterium extorquens, Methylobacillus flagellates, Methylobacillus glycogenes, Methylobacillus pratensis, Hydrogenobacter thermophilus, Acidomonas methanolica, Methylococcus capsulatus, Methylophilus methylotrophus, Methylophilus flavus, Methylophilus luteus, Methylacidiphilum infernorum, Methylibium petroleiphilum, Hydrogenibacillus schlegelii, Lactococcus. sp. Lactobacillus. sp., Bacillus sp. Geobacillus sp. Corynebacterium. sp. Klebsiella, oxytoca, Ralstonia. sp., Alcaligenes. sp. Cupriavidus. sp.

In one embodiment, the host is not E. coli.

Sources of encoding nucleic acids for use in the present invention can include any species where the encoded gene product is capable of catalysing the referenced reaction. Such species include both prokaryotic and eukaryotic organisms including, but not limited to, bacteria, including archaea and eubacteria, and eukaryotes, including yeast, plant, insect, animal and mammal, including human.

Exemplary sources of nucleic acids are described herein. However, with the large number of complete genome sequences available, the identification of genes encoding the requisite 1,3-BDO biosynthetic activity (e.g. the aldolase-type enzymes described herein) for one or more genes in related or distant species, including for example, homologs, orthologs, paralogs and non-orthologous gene displacements of known genes, and the interchange of genetic alterations between organisms is routine and well known to those skilled in the art, and can be carried out in the present context in the light of the teaching herein.

Consequently, in the light of the present disclosure, the metabolic modifications enabling biosynthesis of 3-hydroxybutanal or downstream product derived therefrom (e.g. 1,3-BDO) described herein with reference to a particular organism such as Moorella thermoacetica can be readily applied to other microorganisms. Those skilled in the art will know that a metabolic modification exemplified in one organism can be applied equally to other organisms.

Those skilled in the art will recognise that whenever a particular protein or nucleic acid is referred to herein e.g. with reference to an accession number or other deposit identification, that a functional variant of that sequence may also be used. Since the present invention is primarily concerned with enzyme activities, it will be appreciated that a functional variant will be one which catalyses the same substrate to product reaction as that catalysed by the enzyme referred to, but has a different sequence.

Non-limiting examples of variants include the following:

(i) Novel, naturally occurring, nucleic acids, isolatable using the recited or referred to sequence. These may include alleles (which will include polymorphisms or mutations at one or more bases), paralogues, isogenes, or other homologous genes belonging to the same families as the relevant enzymes. Also included are orthologues or homologues from different microbial or other species.

Thus, included within the scope of the present invention are uses of nucleic acid molecules which encode amino acid sequences which are homologues of the genes referred to herein. Homology may be at the nucleotide sequence and/or amino acid sequence level, as discussed below. A homologue from a different species or strain encodes a product which causes a phenotype similar to that caused by the recited sequence.

(ii) Artificial nucleic acids, which can be prepared by the skilled person in the light of the present disclosure. Such derivatives may be prepared, for instance, by site directed or random mutagenesis, or by direct synthesis. Preferably the variant nucleic acid is generated either directly or indirectly (e.g. via one or more amplification or replication steps) from an original nucleic acid having all or part of the sequence referred to herein.

Changes may be desirable for a number of reasons. For instance they may introduce or remove restriction endonuclease sites or alter codon usage.

Alternatively changes to a sequence may produce a derivative by way of one or more (e.g. several) of addition, insertion, deletion or substitution of one or more nucleotides in the nucleic acid, leading to the addition, insertion, deletion or substitution of one or more (e.g. several) amino acids in the encoded polypeptide.

Other desirable mutations may be random or site directed mutagenesis in order to alter or evolve the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three dimensional structure. In regions which are critical in determining the peptides conformation or activity such changes may confer advantageous properties on the polypeptide. Indeed, changes such as those described above may confer slightly advantageous properties on the peptide e.g. altered stability or specificity.

The term ‘variant’ nucleic acid as used herein encompasses all of these possibilities. When used in the context of polypeptides or proteins it indicates the encoded expression product of the variant nucleic acid.

Some of the aspects of the present invention relating to variants will now be discussed in more detail.

Sequence identity may be assessed as using BLASTp (proteins) or Megablast (nucleic acids) from NCBI (http://www.ncbi.nlm.nih.gov/blast/Blast.cgi) using default settings, as used in the Examples.

Variants of the sequences disclosed herein preferably share at least 55%, 56%, 57%, 58%, 59%, 60%, 65%, or 70%, or 80% identity, most preferably at least about 90%, 95%, 96%, 97%, 98% or 99% identity. Such variants may be referred to herein as “substantially homologous”.

Nucleic acid fragments may encode particular functional parts of the enzyme (i.e. encoding a biological activity of it). Thus the present invention provides for the production and use of fragments of the full-length polypeptides disclosed herein, especially active portions thereof. An “active portion” of a polypeptide means a peptide which is less than said full length polypeptide, but which retains its essential biological activity.

Generally speaking, those skilled in the art are well able to construct vectors and design protocols for the recombinant genetic manipulations described herein. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

A “vector” as used herein need not include a promoter or other regulatory sequence, particularly if the vector is to be used to introduce nucleic acid into cells for recombination into the genome.

However for expression purposes the nucleic acid in the vector will typically be under the control of, and operably linked to, an appropriate promoter or other regulatory elements for transcription in a microbial host cell. It may include a native promoter. In the case of cDNA this may be under the control of an appropriate promoter or other regulatory elements for expression in the host cell.

By “promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter. In one embodiment, the promoter is an inducible promoter. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

The present disclosure teaches how pathways may be engineered into an organism by selection of the appropriate enzymes, cloning their corresponding genes into a production host, optimising the stability and expression of these genes, attenuation or functional deletion of the competitive pathways, optimising fermentation conditions for the genetically engineered strain to produce the desired product, and assaying for product formation following fermentation.

The term “heterologous” is used broadly herein to indicate that the gene/sequence of nucleotides in question (e.g. encoding an aldolase) has been introduced into a host cell or an ancestor thereof, using genetic engineering, i.e. by human intervention. Nucleic acid heterologous to a host cell will be non-naturally occurring in cells of that type, variety or species. Thus the heterologous nucleic acid may comprise a coding sequence of or derived from a microorganism, placed within a different microorganism. A further possibility is for a nucleic acid sequence to be placed within a cell in which it or a homologue is found naturally, but wherein the nucleic acid sequence is linked and/or adjacent to nucleic acid which does not occur naturally within the cell, such as operably linked to one or more regulatory sequences, such as a promoter sequence, for control of expression.

“Transformed” in this context means that the nucleotide sequences of the heterologous nucleic acid alter one or more of the cell's characteristics and hence phenotype e.g. with respect to 3-hydroxybutanal or downstream product derived therefrom (e.g. 1,3-BDO).

“Nucleic acid” when used in the present invention may include cDNA, RNA, genomic DNA and modified nucleic acids or nucleic acid analogs (e.g. peptide nucleic acid). Where a DNA sequence is specified, e.g. with reference to a figure, unless context requires otherwise the RNA equivalent, with U substituted for T where it occurs, is encompassed. Nucleic acid molecules according to the present invention may be provided isolated and/or purified from their natural environment, in substantially pure or homogeneous form, or free or substantially free of other nucleic acids of the species of origin, and double or single stranded. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Nucleic acids may comprise, consist, or consist essentially of, any of the sequences discussed hereinafter.

In the methods herein any shuttle vectors available for Gram-positive bacteria that carry at least one nucleotide sequence homologous to one gene encoding the desired enzyme can be employed for transformation of M. thermoacetica or other microorganism of interest.

An expression plasmid is obtained by inserting at least a gene responsible for replication of the plasmid in Gram-positive and more specifically in Clostridia species or acetogens. The plasmid capable of introducing the desired gene into an acetogen is not particularly limited as long as it contains at least a gene responsible for replication and amplification in acetogenic bacteria. Specific examples thereof include pAK201 (Kim, A. and Blashek, H. P., Appl. Environ. Microbiol. 55 (2):360-365 (1988), pHB101 (Blaschek H. P. et. al, J. Bacterial. 147(1):262-266 (1981)), any of the series modular plasmids pMTL8000 (Heap, J. T. et al., J. Microbiol. Methods 78:79-85 (2009), pMS1, pMS2, pMS3, pMS4, pKV12 (Staetz, M. et al, Appl. Environ. Microbiol. 1033-1037 (1994), pUB110 (McKenzie et al., 1984), pIMP1 (Mermelstein, L et al. 1992), pITF (Dong, H. et al. 2010). A further example of a plasmid known to be suitable for use in acetogens is pMTL80000 (Kopke, M. et al., Appl. Environ. Microbiol. 3394-3403, 2014) available from The University of Nottingham, United Kingdom.

Novel shuttle vectors, which are chimeras of pUB110 or any of the above mentioned plasmids and a general E. coli cloning vectors such as pUC19 (Yanisch-Perron, C. et al., Gene 33:103-119 (1985)) or pBluescript II SK (+/−) can be easily generated and tested. These chimera plasmids are propagated in E. coli for plasmid isolation and employed for the genetic engineering work of M. thermoacetica or another acetogen or Gram-positive bacteria which is naturally sensitive towards the antibiotic gene expressed by the plasmid. If needed, sub-cloning can be employed to replace the antibiotic resistance cassettes on the existing plasmids with suitable ones based on the antibiotic sensitivity of the target organism. Standard techniques for DNA amplification using a high-fidelity DNA polymerase and molecular sub-cloning, including restriction enzyme digestion, ligation and E. coli transformation can be used for engineering of the plasmids (Sambrook, 1989).

The antibiotic resistance of M. thermoacetica has been tested in liquid cultures and on plates and selection conditions have been identified. In one embodiment kanamycin and chloramphenicol may be utilised as antibiotic markers for selection of the genetic engineered M. thermoacetica strains.

The operon or one gene of the operon encoding the required activity can be ligated into the multiple cloning site between two convenient restriction sites.

In order to achieve optimum gene(s) expression for the heterologous genes introduce, the heterologous genes can be codon optimised for the target organism with techniques well known to those skilled in the art.

To ease the detection and quantification of the gene product(s) expression, an N- or C-terminus tag sequence can be added to the gene sequences cloned as understood by those skilled in the art.

Many Clostridia species have been successfully transformed with prior methylated DNA vectors. The methylation of the transformable DNA protects it from being degraded by the host. In vivo methylation of the transformable DNA is achieved by its propagation in methylation E. coli strains such as Top10 (pAN2) (Kuit et al., Appl. Microbiol. Biotechnol. 94:729-741 (2012)).

Heterologous (or exogenous, the terms are used interchangeably) gene(s) can be introduced into the chosen host cell, exemplified herein by M. thermoacetica and Acetobacterium woodii, using techniques well known in the art including, but not limited to, conjugation, electroporation, chemical transformation, transduction, transfection and electrofusion. For electroporation and conjugation, published protocols of Clostridium perfringens, Clostridum. acetobutylicum, Clostridium. cellulolyticum and Acetobacterium woodii may be used.

In some embodiments it may be desired to target or inactivate genes in the host microbial cell, for example to increase flux of target metabolic intermediates and\or 1,3-BDO, or divert metabolic pathways away from biomass generation. An example is to minimise loss of pyruvate away from a 3-hydroxybutanal pathway.

To permanently inactivate genes in M. thermoacetica or other acetogens, a plasmid can be constructed for gene deletion by integrational mutagenesis or gene replacement techniques well known in the art. Integrational mutagenesis and gene replacement can selectively inactivate undesired genes from host genomes. Such methods have been developed and successfully used to create metabolically engineered mutants of Clostridial strains (Green et al., 1996). In this technique, a fragment of the target gene is cloned into a non-replicative vector with a selection marker, resulting in the non-replicative integrational plasmid. The partial gene in the non-replicative plasmid can recombine with the internal homologous region of the original target gene in the parental chromosome (double crossover), which results in the insertional inactivation of the target gene, ldh locus in this particular example. The use of gene replacement (by double recombination) is preferred to insertional inactivation (single recombination) since it permits the generation of more stable engineered strains, without the need to maintain selection of vectors. An example describing a double crossover in an acetogen is shown in Example 5.

Using this technique, in the same manner non-natural microorganisms can be generated having complete or partial deletion of one, two, three, four, five, or more genes in order to remove competitive pathways.

Reduction of expression of the target genes can also be used as an alternative to gene disruption. This may be achieved using expression of antisense RNA for the target gene, which will inhibit but not completely abolish gene expression. The antisense RNA system serves as a convenient approach of gene knock-down of a desired gene with the advantage that it can reduce expression of genes for which complete inactivation could be damaging or lethal to the organism.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al, (1988) Nature 334, 724-726.

The complete sequence corresponding to the coding sequence (in reverse orientation for anti-sense) need not be used. For example fragments of sufficient length may be used.

It is a routine matter for the person skilled in the art to screen fragments of various sizes and from various parts of the coding sequence to optimise the level of anti-sense inhibition. It may be advantageous to include the initiating ATG codon, and perhaps one or more nucleotides upstream of the initiating codon. A further possibility is to target a conserved sequence of a gene, e.g. a sequence that is characteristic of one or more genes, such as a regulatory sequence.

The sequence employed may be about 500 nucleotides or less, possibly about 400 nucleotides, about 300 nucleotides, about 200 nucleotides, or about 100 nucleotides. It may be possible to use oligonucleotides of much shorter lengths, 14-23 nucleotides, although longer fragments, and generally even longer than about 500 nucleotides are preferable where possible, such as longer than about 600 nucleotides, than about 700 nucleotides, than about 800 nucleotides, than about 1000 nucleotides or more.

It may be preferable that there is complete sequence identity in the sequence used for down-regulation of expression of a target sequence, and the target sequence, although total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a variant of such a sequence in the terms described above. The sequence need not include an open reading frame or specify an RNA that would be translatable.

The transformation, expression and application of antisense RNA inhibition tools have been demonstrated for mesophilic Clostridia such as: Clostridium acetobutylicim (Desai R. et al. Appl. Environ & Eviron Microbiol. 65(3):936-945 (1999)) Fierro-Monti I P et. al., J Bacteriol. 174(23):7642-7647 (1992)) and Clostridium cellulolyticum (Perret S, et al., Mol. Microbiol. 51(2):599-607 (2004)) as well as for termophiles such as Thermus thermophilus (Moreno, R. et al., J. Bacteriol., 7804-7806(2004) and may be applied herein.

An attractive approach for down-regulation expression of a target gene is to replace the native promoter with a less active promoter for example one from another gene. This can be achieved by double-recombination/gene replacement techniques well known in the art. Alternatively, expression can be reduced by altering the ribosome binding site or the spacing between the RBS and the translation initiation start codon, or using a less efficient start codon.

The results of these studies permit for phenotypic characterisation of the mutants generated as well as allow genetic engineering of M. thermoacetica or other acetogens. Further optimisation can be performed to develop genetic systems by varying methods, plasmids and conditions to achieve optimum results. Specifically, the metabolic modifications enabling biosynthesis of 1,3-BDO described herein with reference to a particular organism such as M. thermoacetica can be readily applied to other microorganisms, including prokaryotic and eukaryotic organisms alike.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, and the abstract appended hereto, inasmuch as it may be used by those skilled in the art to carry out the invention, is hereby specifically incorporated herein by cross-reference.

### FIGURES

FIG. 1. Example of chemical transformation of 1,3-butanediol into industrially important chemicals including butadiene and methylethyl ketone. Ichikawa et al., J. Molecular Catalysis A-Chemical, 256:106-112 (2006)

FIG. 2. Shows the Wood Ljungdahl pathway for synthesis of 3 acetyl CoA (3 acetate), from gaseous carbon sources with or without methanol, showing the entry point for methanol. Associated equations are: 4CH₃OH+2CO₂→3CH₃COOH; 12CO+6H₂O→3CH₃COOH+6CO₂; 12H₂+6CO₂→3CH₃COOH+6H₂O. The Wood Ljungdahl pathway can also fix CO₂ derived from the glycolytic pathway (pyruvate decarboxylation) using reducing equivalents derived from glycolysis and pyruvate decarboxylation.

FIG. 3. Shows metabolic pathways (route 1, 2, 3, 4, 5 and 6) for the synthesis of 1,3-butanediol from the central metabolic intermediates acetyl CoA or pyruvate, via the common intermediate acetaldehyde. Enzyme activities required to catalyse these steps are listed as Activity A, B, C, D, E, F, G, and H. Example gene sequences coding for these activities can be found in Tables 1, 2, 3, 4, 5, 6, and 7.

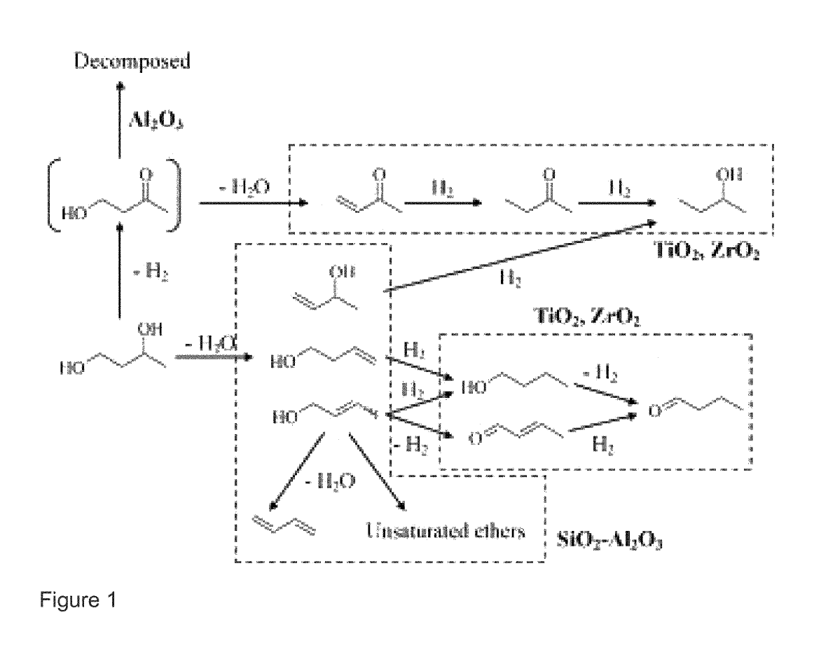

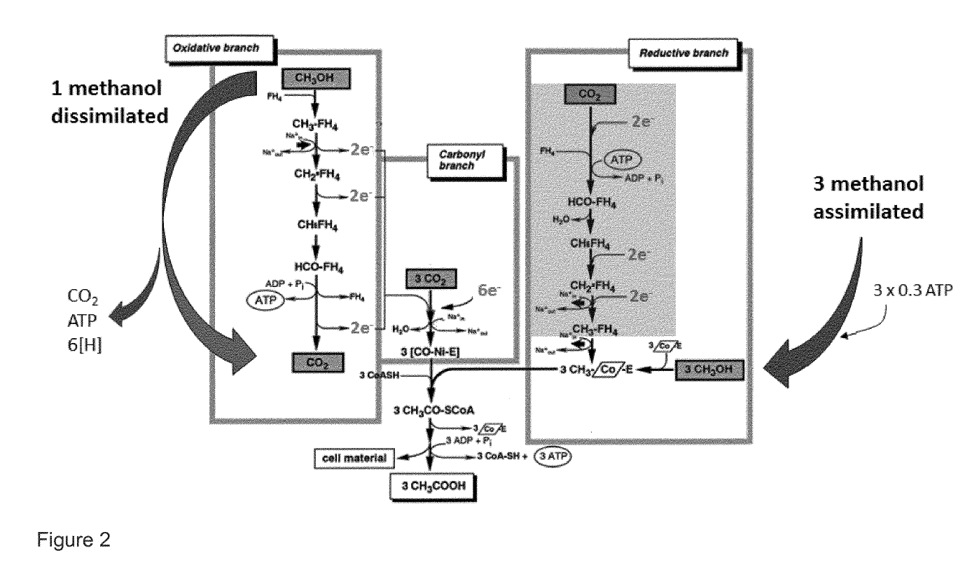

Route 1 proceeds from acetyl CoA through acetate (a natural product of acetogenic microorganisms) to acetaldehyde via carboxylic acid reductase activity, for example, EC 1.2.7.5 or EC. 1.2.99.6, ATP or ferredoxin driven or EC 1.2.1.30 or EC 1.2.1.3.

Route 2 involves direct synthesis of acetaldehyde from acetyl CoA using an aldehyde dehydrogenase (acylating), for example, acetaldehyde dehydrogenase EC 1.2.1.10.

Route 3 involves the conversion of pyruvate to acetaldehyde via acetyl CoA using enzymes such as EC 1.2.7.1 or EC 1.2.1.51 or EC 1.2.4.1 and EC 1.2.1.10.

Route 4 involves the conversion of pyruvate to acetaldehyde, directly via pyruvate decarboxylase (EC 4.1.1.1).

Route 5 involves the conversion of acetyl CoA to acetaldehyde via pyruvate using enzymes such as EC 1.2.7.1 and EC 4.1.1.1.

Route 6 involves the conversion of acetate to acetaldehyde via acetyl CoA using enzymes such as EC 6.2.1.1 or EC 2.8.3.8 and EC 1.2.1.10.

Two molecules of acetaldehyde are condensed to form 3-hydroxybutanal using an aldolase capable of accepting an aldehyde as both the acceptor and donor in an aldol condensation, for example, deoxyribose phosphate aldolase (DERA, EC 4.1.2.4). 3-Hydroxybutanal is reduced to 1,3-butanediol by an alcohol dehydrogenase or aldehyde reductase, for example, using enzymes categorised in EC 1.1.1.1, EC 1.1.1.2, EC 1.1.1.72 or EC 1.1.1.265 or EC 1.1.1.283.

FIG. 4. Shows the RuMP pathway and its association with the TCA cycle (modified from Appl. Environ Microbiol. 2003 69, 3986). Pyruvate is the primary product of the RuMP pathway which is converted to acetyl CoA prior to entry to the TCA cycle. Either pyruvate or acetyl CoA can be converted directly to the common intermediate acetaldehyde thereby supplying substrate for a DERA type aldolase capable of accepting acetaldehyde as both the donor and acceptor in an aldol condensation for synthesis of 1,3-butanediol.

FIG. 5. Shows the Wood Ljungdahl pathway. Either pyruvate or acetyl CoA can be converted directly to the common intermediate acetaldehyde thereby supplying substrate for a DERA type aldolase capable of accepting acetaldehyde as both the donor and acceptor in an aldol condensation for synthesis of 1,3-butanediol. Modified from Fung Min Liew, Michael Köpke and Sean Dennis Simpson (2013). Gas Fermentation for Commercial Biofuels Production, Liquid, Gaseous and Solid Biofuels—Conversion Techniques, Prof. Zhen Fang (Ed.), ISBN: 978-953-51-1050-7, InTech, DOI: 10.5772/52164. Acetate derived from acetyl CoA can also be directly reduced to acetaldehyde for supply to the aldolase.

FIG. 6. Shows the reverse TCA cycle. Either pyruvate or acetyl CoA can be converted directly to the common intermediate acetaldehyde thereby supplying substrate for a DERA type aldolase capable of accepting acetaldehyde as both the donor and acceptor in an aldol condensation for synthesis of 1,3-butanediol. Modified from Mar. Drugs. 2011, 9, 719.

FIG. 7. Shows the serine cycle. Acetyl CoA can be converted directly to the common intermediate acetaldehyde supplying substrate for a DERA type aldolase capable of accepting acetaldehyde as both the donor and acceptor in an aldol condensation for synthesis of 1,3-butanediol. Central metabolism also converts PEP (phosphoenol pyruvate) into pyruvate which can be decarboxylated to acetaldehyde as described previously.

FIG. 8. Shows the condensation of acetaldehyde catalysed by deoxyribose phosphate aldolase (DERA). The first condensation product 3-hydroxybutanal accumulates without further condensation, or is subject to a second acetaldehyde addition depending on the enzyme and the reaction conditions.

FIG. 9. Shows the Cavin cycle linked to sugar synthesis (or utilisation) and or conversion to pyruvate or acetyl CoA directly. Either pyruvate or acetyl CoA can be converted directly to the common intermediate acetaldehyde thereby supplying substrate for a DERA type aldolase capable of accepting acetaldehyde as both the donor and acceptor in an aldol condensation for synthesis of 1,3-butanediol.

FIG. 10. Shows Acetobacterium woodii grown on an agar plate containing 0.1 g/L MUG (4-Methylumbelliferyl-β-D-glucopyranosiduronic acid) demonstrating successful expression of a heterologous gene in an acetogen. This system can also act as a reporter to confirm expression of other heterologous genes.

### Key.

A1: Colony 1 of A woodii carrying plasmid pEP55
A2: Colony 2 of A woodii carrying plasmid pEP55
B1: Colony 1 of A woodii carrying plasmid pEP56
B2: Colony 2 of A woodii carrying plasmid pEP56
C: Negative Control, A. woodii carrying the pEP plasmid expressing an unrelated gene

FIG. 11. Cloning strategy to construct an A. woodii LDH knockout mutant by replacing the LDH gene with an Erythromycin resistance marker.

FIG. 12. Cloning strategy to construct an A. woodii LDH knockout mutant by disrupting the LDH gene via single cross-over recombination event and integration of the complete plasmid.

FIG. 13. Growth of A. woodii wildtype and A. woodii mutants in the presence of 20 mM Fructose and 40 mM DL-Lactate. Aw=A. woodii wildtype, Plasmid=A. woodii transformant harboring plasmid pUC19-Ery-pAMβ1, dLDH=double cross-over LDH knockout. SR=Single cross-over LDH knockout.

FIG. 14. Utilization of Fructose and Acetate production by A. woodii wildtype and A. woodii mutants.

Aw=A. woodii wild type, P=A. woodii transformant harboring plasmid pUC19-Ery-pAMβ1, dLDH=double cross-over LDH knockout. SR=Single cross-over LDH knockout.

FIG. 15. Utilization of Lactate and Acetate production A. woodii wildtype and A. woodii mutants.

Aw=A. woodii wild type, P=A. woodii transformant harboring plasmid pUC19-Ery-pAMβ1, dLDH=double cross-over LDH knockout. SR=Single cross-over LDH knockout.

FIG. 16. Representative mass spectrometry data for the product 1,3-butanediol produced from various pathway combinations incorporating DERA enzymes

FIG. 17. Examples of downstream products obtainable from 3-hydroxybutanal.

### EXAMPLES

Methods and Materials—Cloning, Expression and Activity Assay for Gene(s) for Engineering into Acetogens to Produce 1,3-Butanediol

The approach to construction of the 1,3-butanediol pathway in a chosen host will depend on the pathway genes already present in the host organism. Those endogenous genes considered suitable for pathway construction may be overexpressed to ensure adequate flux through the pathway to 1,3-butanediol.

Metabolic engineering steps required to generate a 1,3-butanediol production strain will depend on whether pyruvate or acetyl CoA or both are selected as the source of acetaldehyde. Subsequent conversion of acetaldehyde is common to all routes. For example, for Route 1, acetaldehyde is derived from acetyl CoA via acetate. Acetate is a natural acetogen product which can accumulate to 10 s grams per litre. For example 44 g/l was obtained from the acetogen Acetobacterium woodii growing on CO₂ and H₂ (Demlar, M. et al. Biotech. Bioeng. 2011, 108, 470). Overexpression of a carboxylic acid reductase, aldehyde ferredoxin oxidoreductase or other enzyme capable of acetate reduction (exemplary sequences given in Table 1) to acetaldehyde in the presence of sufficient reducing equivalents and ATP (if appropriate), allows conversion to acetaldehyde. Other than production of biomass for the fermentation, in this example it is desirable to optimise all carbon flux to acetate or acetyl CoA. Accumulation of by-products which are not required for biosynthesis, such as lactate is avoided by knockout of the respective genes e.g. lactate dehydrogenase (Example 5) overproduction of metabolites required for cell synthesis such as malate or fumarate is avoided by adequate, balanced, carbon flux to avoid bottle necks.

Direct conversion of acetyl CoA to acetaldehyde using acetaldehyde dehydrogenase (overexpression of an endogenous enzyme, or introduction of, for example eutE, Table 2) can operate in the absence of acetate accumulation (Route 2) or alongside acetate accumulation where flux is directed to acetaldehyde directly or via acetate. The route chosen may be influenced by the energetics requirement of organism which can be related to the feedstock provided. It is most preferable to convert a primary central metabolic intermediate to acetaldehyde directly. If the bioenergetics allow loss of ATP synthesis from acetyl CoA conversion to acetate, acetate accumulation can be prevented in an acetogen by knockout of one or more phosphotransacetylase (pta) or acetate kinase (ack) genes (Example 6 and 8). Furthermore, acetate accumulation may be prevented by natural regulation, or by mutation which directs flux away from acetate synthesis while maintaining Wood Ljungdahl pathway activity. For example growth of the acetogen Moorella thermoacetica (renamed from C. thermoaceticum) on CO and methanol in the presence of nitrate led to no acetate accumulation due to repression of key Wood Ljungdahl related gene expression (Seifritz, C. et al. J. Bacteriol. 1993, 175, 8008). In that example, sufficient ATP appeared to be provided from nitrate respiration. Acetyl CoA can also be converted to acetaldehyde via pyruvate (Route 5) using pyruvate synthase (EC 1.2.7.1, Table 3). In this example it is particularly desirable to avoid loss of carbon flux to products derived from pyruvate other than acetaldehyde (for example targeting of LDH may be desired), Example 5).

If pyruvate is the primary central metabolic intermediate, it is preferable to convert pyruvate to acetaldehyde directly (Route 4) via decarboxylation using example sequences in Table 4 and to optimise the flux by targeting of undesired pathways (for example LDH or pyruvate formate lyase). However, it may alternatively be preferred to allow conversion of pyruvate to acetyl CoA, Route 3 (the natural metabolic route prior to entry to the TCA cycle) or by using gene sequence examples shown in Table 3. It is desirable that the maximum amount of acetaldehyde be converted to 3-hydroxybutanal via an overexpressed endogenous or heterologous DERA (example sequences are shown in Table 6). Hence, loss to oxidation or reduction products (acetate or ethanol) should be avoided by knockout of undesired genes, for example, short chain alcohol dehydrogenases highly active on acetaldehyde, or non-acetylating acetaldehyde dehydrogenase (e.g. EC 1.2.1.5). Reduction of 3-hydroxybutanal is achieved by overexpression of an endogenous, or introduction of a heterologous alcohol dehydrogenase or aldehyde reductase which shows preference for C4 aldehydes (3-hydroxybutanal) relative to C2 aldehydes (acetaldehyde) e.g Example 9. Such examples are discussed above and example sequences shown in Table 7.

The introduction of a heterologous gene into an acetogen is described in Example 7, this method can be cross applied to the introduction of any heterologous gene, for example, a gene within a 1,3-butanediol pathway.

### Example 1—Routes for Acetaldehyde and 1,3-BDO Synthesis from Central Metabolites

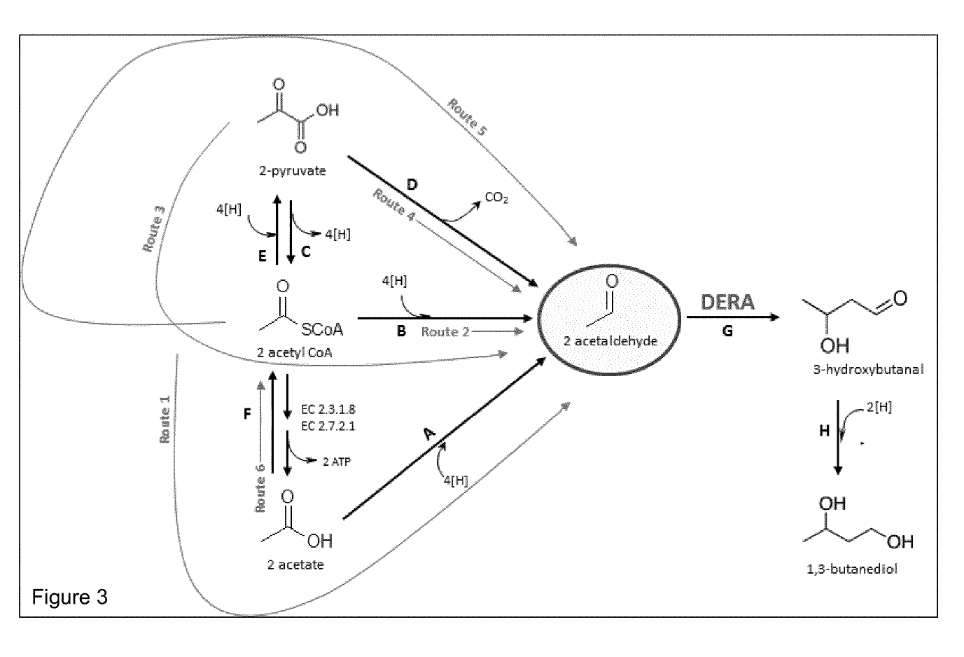

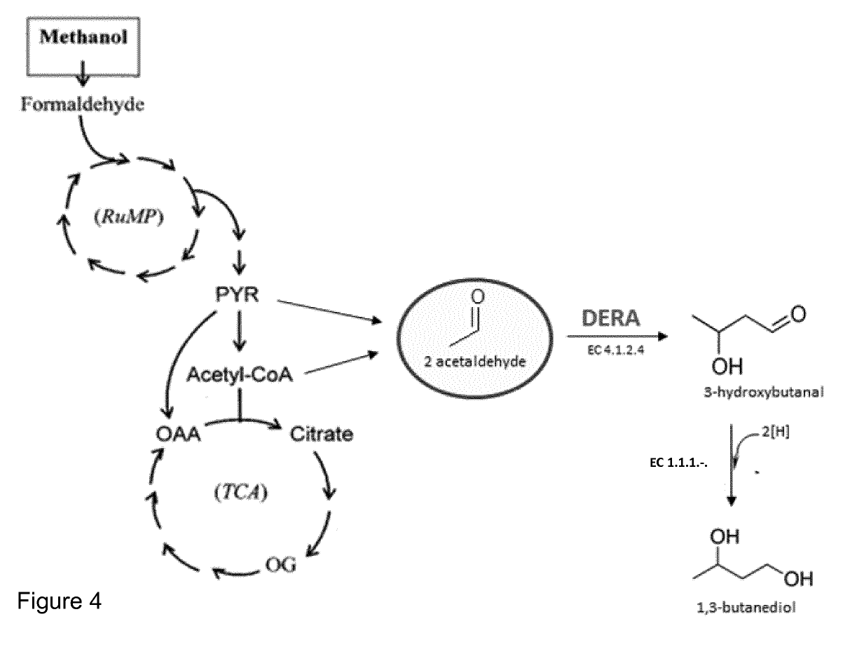

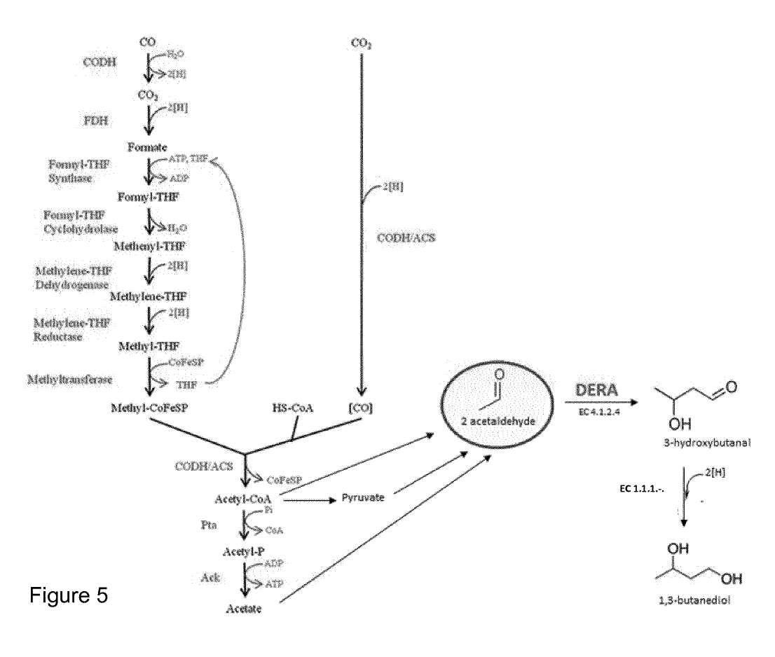

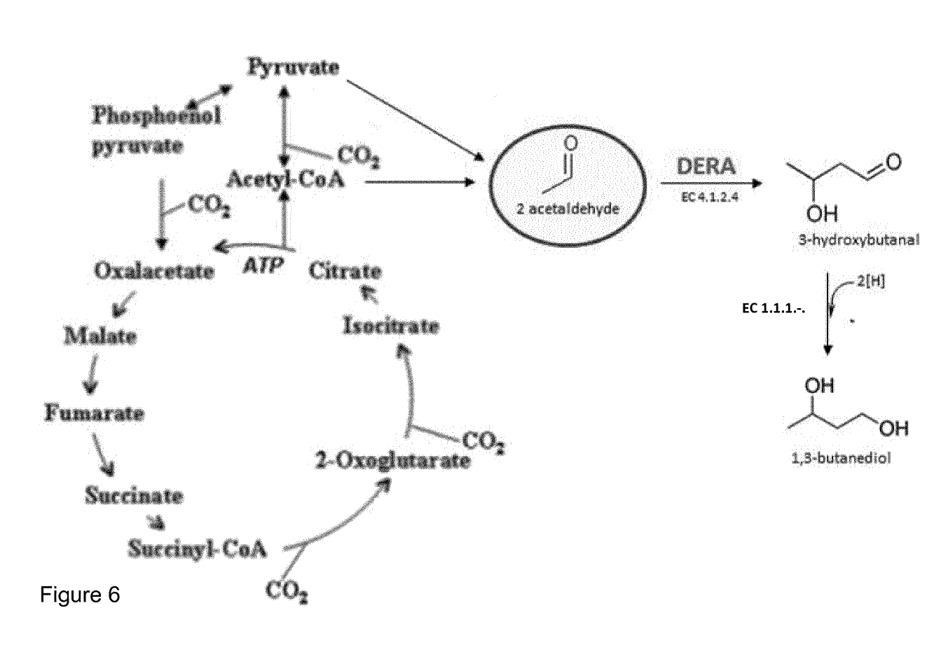

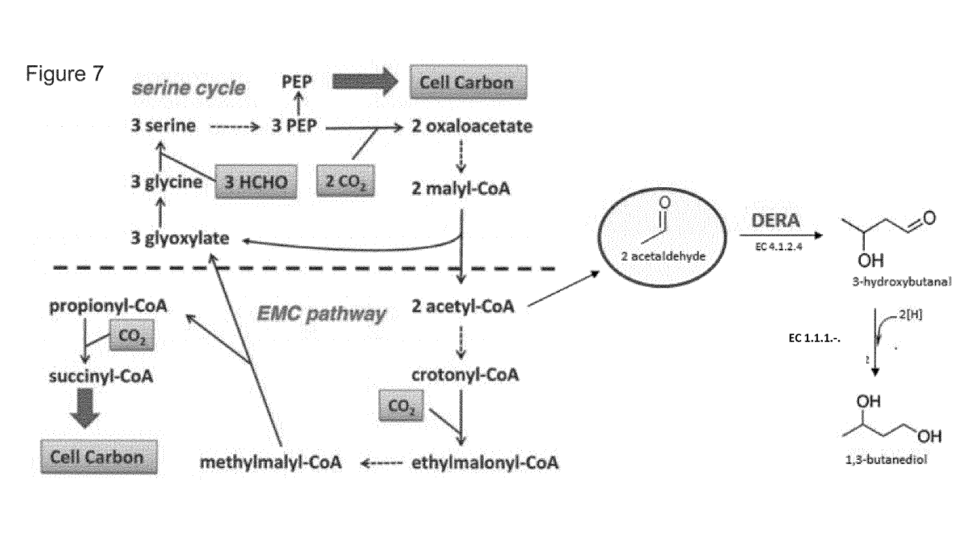

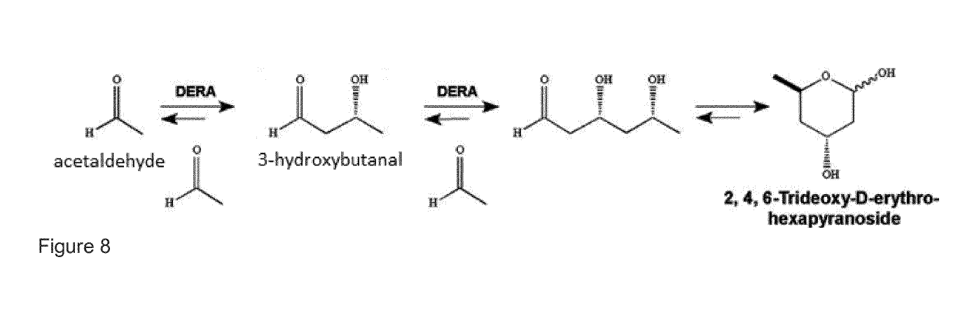

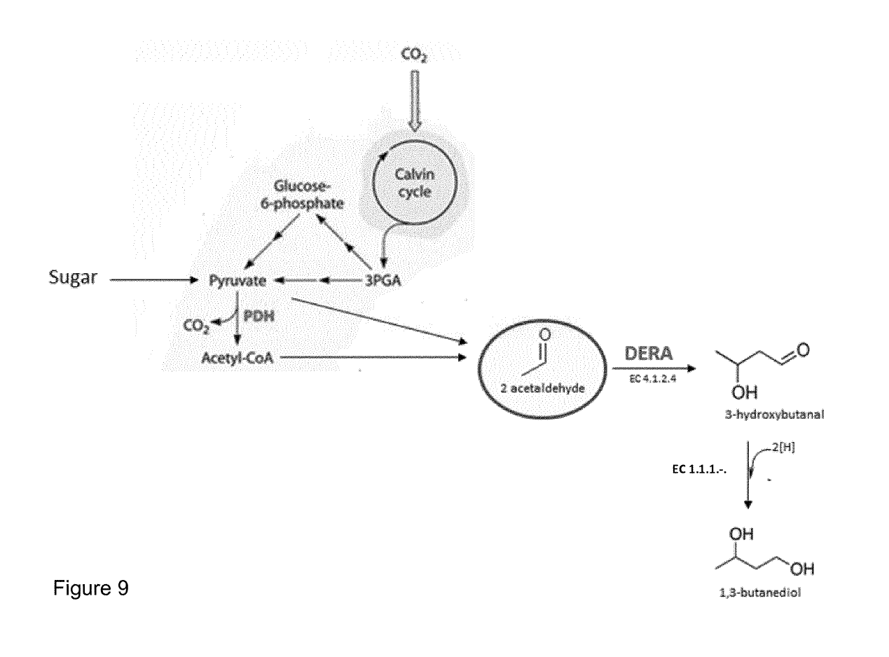

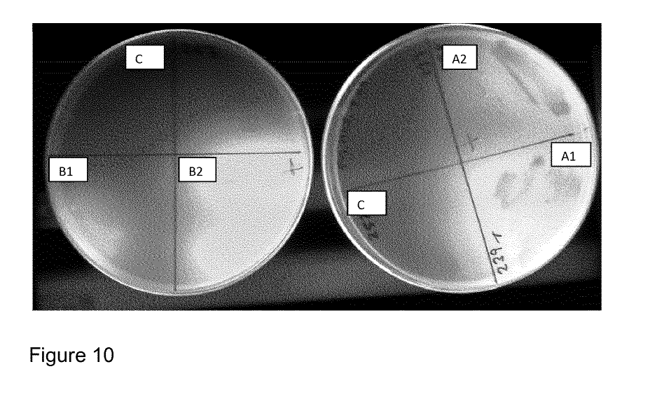

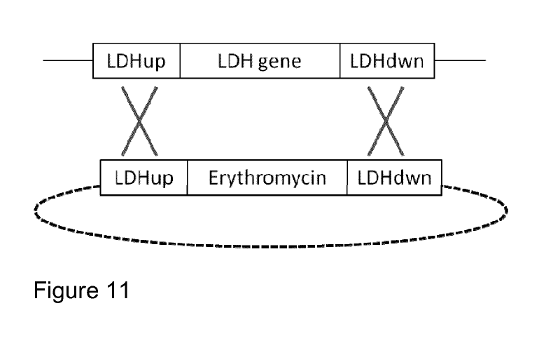

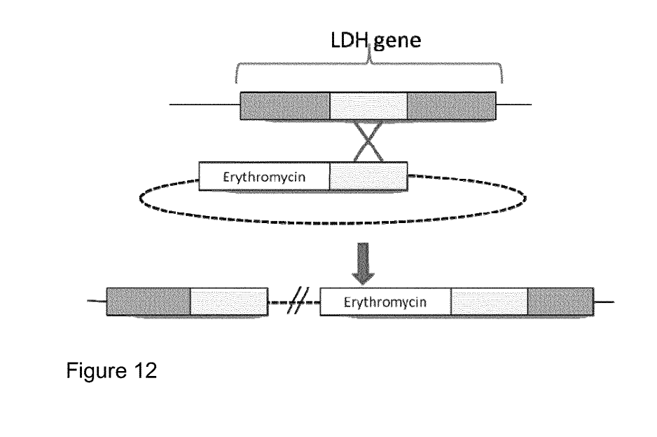

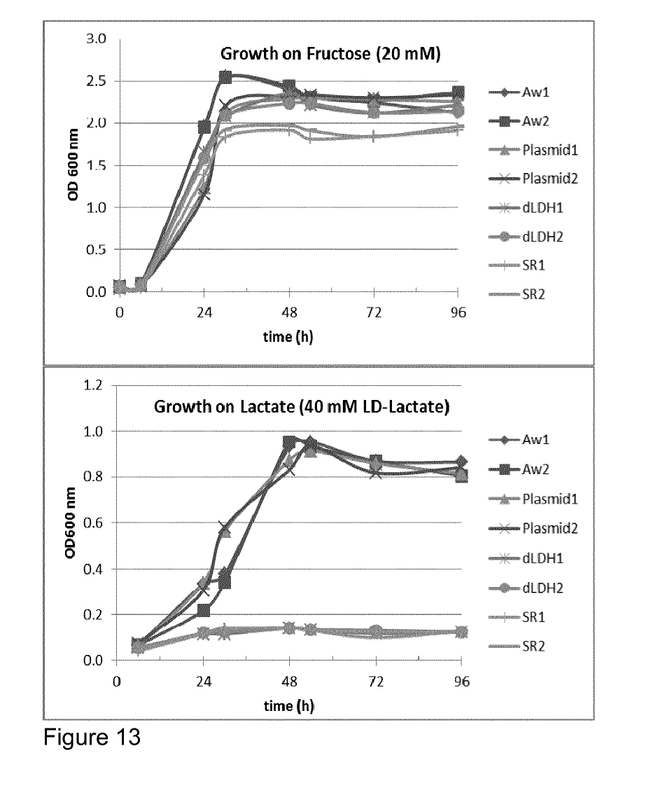

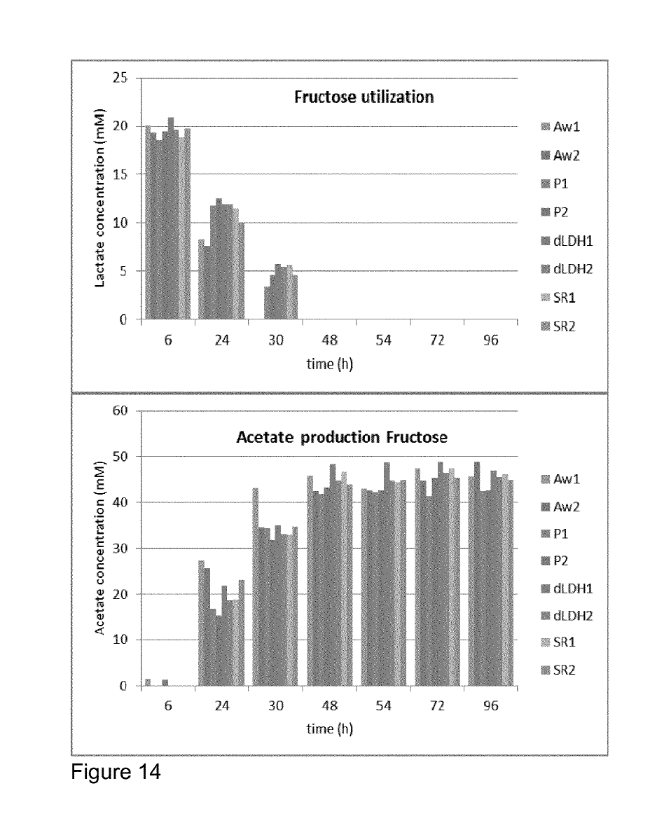

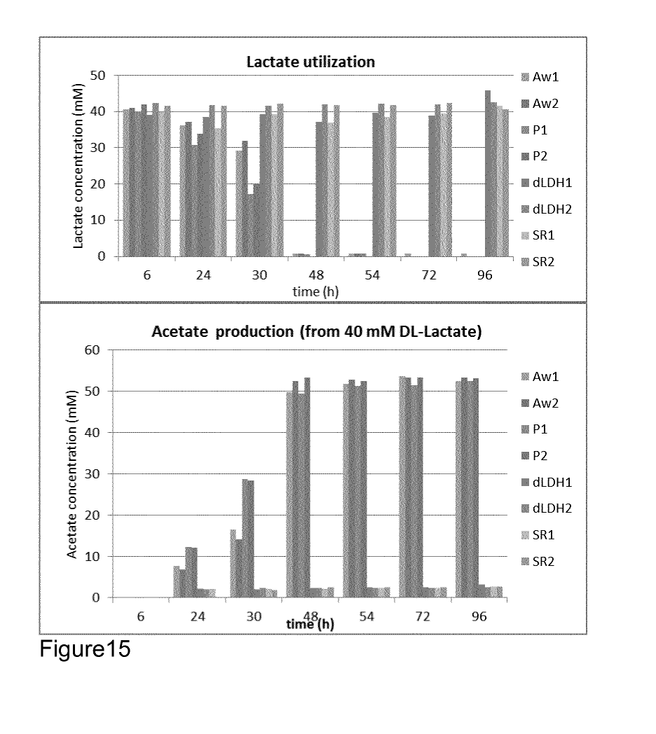

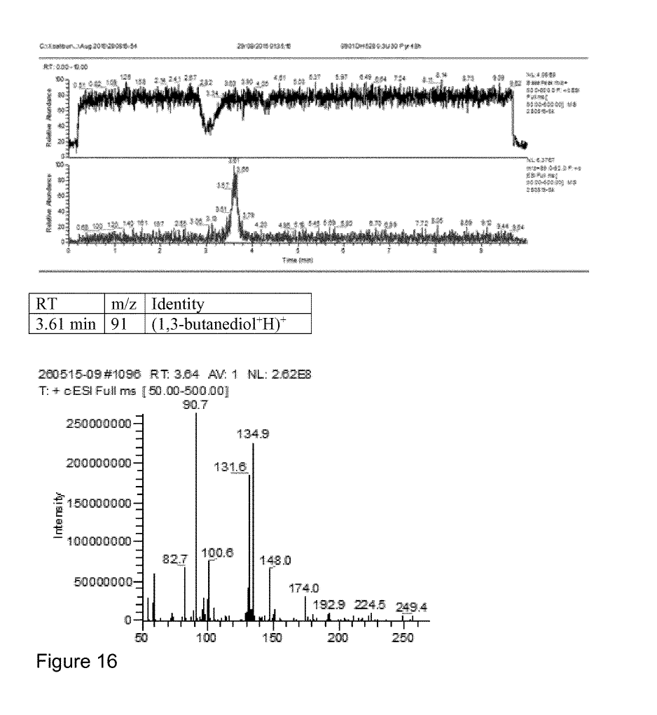

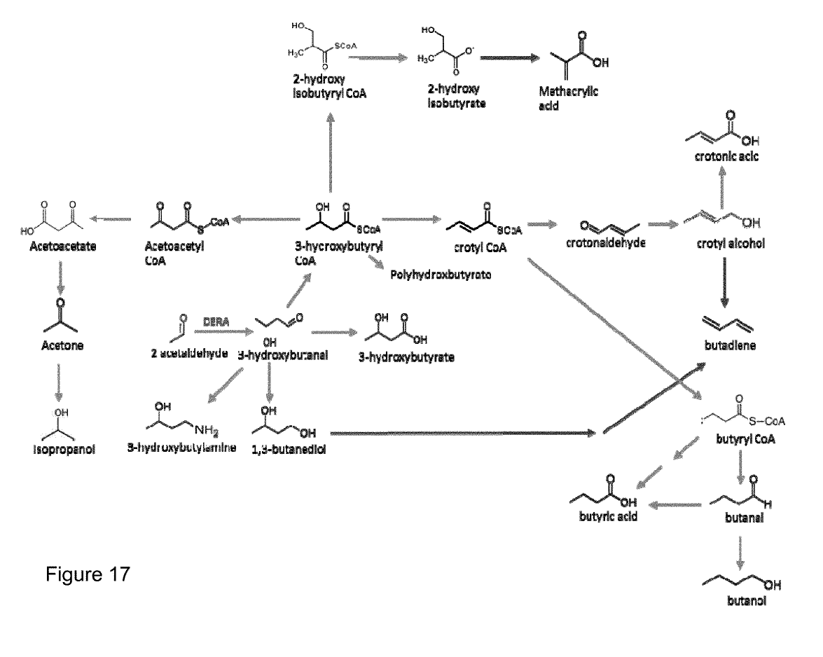

The overall conversion of acetyl CoA to 1,3-butanediol is accomplished in either 3 or 5 steps depending on the route taken (FIG. 3) and in 1 or 3 steps to the common pathway intermediate acetaldehyde. Other products obtainable via acetaldehyde and 3-hydroxybutanal are described above (see also FIG. 17).

The overall conversion of pyruvate to 1,3-butanediol is accomplished in 3 or 4 steps depending on the route taken (FIG. 3) and in 1 or 2 steps to the common pathway intermediate acetaldehyde.

The two steps from acetaldehyde to 1,3-butanediol are common to all 1,3,-butanediol synthetic routes.

The description of the pathways is provided as routes for acetaldehyde synthesis (Route 1,2, 3, 4, 5 and 6) and the subsequent conversion of acetaldehyde to 1,3-butanediol via the aldol condensation catalysed by DERA.

### Route 1—Conversion of Acetyl CoA to Acetaldehyde Via Acetate

Acetogens naturally produce acetate in high yield from sugars, or C1 feedstocks (syngas, CO₂/H₂, CO₂ and methanol) via conversion of acetyl CoA derived from the Wood Ljungdahl pathway. Yields are typically approximately 80% of theoretical or greater, for example, A. E. Bainotti et al., 1988. Journal of fermentation and bioengineering, 85(2), 223-229. Although it is anticipated that even higher yields may be achievable, for example, via modification of the Wood Ljungdahl pathway which converts CO₂, H₂, CO, or methanol to acetyl CoA or via optimisation of the growth medium. Fundamentally, in acetogens the general fate of acetyl CoA is either to go towards formation or maintenance of biomass, or synthesis of acetate which generates ATP. As the Wood Ljungdahl pathway requires an ATP, in most cases (depending on the growth conditions), acetate synthesis is required in order to balance the energy needs of the system. Acetate is a major natural product of most acetogens.

Acetate can be reduced to acetaldehyde using a carboxylic acid reductase enzyme. Such enzyme activity mainly uses either reduced ferredoxin (aldehyde ferredoxin oxidoreductase) or ATP to drive the thermodynamically unfavourable reduction of a carboxylic acid moiety and tend to be classified in EC 1.2.7.5, EC 1.2.1.30, EC 1.2.99.6. or EC 1.2.1.3. The term carboxylic acid reductase and aldehyde oxidoreductase are used interchangeably in the literature. Aldehyde dehydrogenase is also used to describe enzymes capable of carboxylic acid reduction.

An example of a well-studied carboxylic acid reductase can be found in Nocardia iowensis which catalyzes the magnesium, ATP and NADPH-dependent reduction of carboxylic acids to their corresponding aldehydes (Venkitasubramanian et al., J. Biol. Chem. 282:478-485 (2007)). This enzyme is encoded by the car gene and was cloned and functionally expressed in E. coli (Venkitasubramanian et al., J. Biol. Chem. 282:478-485 (2007)). Expression of the npt gene product improved activity of the enzyme via post-translational modification. The npt gene encodes a specific phosphopantetheine transferase (PPTase) that converts the inactive apo-enzyme to the active holo-enzyme. The natural substrate of this enzyme is vanillic acid, and the enzyme exhibits broad acceptance of aromatic and aliphatic substrates as small as lactic acid (Venkitasubramanian et al., in Biocatalysis in the Pharmaceutical and Biotechnology Industries, ed. R. N. Patel, Chapter 15, pp. 425-440, CRC Press LLC, Boca Raton, Fla. (2006)). Activity towards acetate was not discussed. However, high activity towards lactate suggests that the enzyme is capable of accepting molecules containing as few as three carbons. Hence, this enzyme may potentially be used for acetate reduction in either its native form or as an evolved enzyme.

A further well studied enzyme is the example from Mycobacterium marinum which has a wild type substrate preference for C6 to C18 acids (Kalim Akhtar, M. et al. PNAS, 2013, 110, 87). Enzymes capable of carboxylic acid reduction may be evolved or mutated as described above to increase activity towards acetate using enzyme evolution techniques common in the art. The griC and griD genes from Streptomyces also code for a carboxylic acid reductase with diverse capability for acid reduction Suzuki et al. 2007. J. Antibiot. 60 (6) 380.

Aldehyde ferredoxin oxidoreductase enzymes use ferredoxin not ATP to drive the carboxylate reduction and are present in many acetogens and other organisms (White, H et al. Biol. Chem Hoppe Seler 1991, 372 (11) 999; White, H and Simon, H. Arch. Microbiol, 1992, 158, 81; Fraisse. L and Simon, H. Arch. Microbiol. 1988, 150, 381; (Basen et. al. 2014. PNAS, 111 (49), 17618). The carboxylic acid reducing enzyme from Moorella thermoacetica has been purified and characterised, White, H. et al. Eur. J Biochem, 1989, 184, 89. Further, using propionate reduction to propionaldehyde, the specific activity was shown to increase when the corresponding aldehyde was removed during the reaction. In the case of application of such an enzyme to this invention, the product acetaldehyde would be continuously removed by the DERA enzyme and would not be expected to accumulate significantly. Huber, C. et al. Arch. Microbiol, 1995, 64, 110.

Example genes for acetate reduction are shown in Table 1. The aldehyde oxidoreductase (AOR) genes CLJU_20110 and CLJU_20210 from Clostridium ljungdahlii are reported to reduce acetate to acetaldehyde, Kopke, M. et al. PNAS, 2010, 107, 15305. Hence, demonstrating the activity of a wild type enzyme towards the target reduction. Various authors have also described conditions under which AOR enzymes are induced in ethanologenic acetogens for synthesis of ethanol from acetate via acetaldehyde, (Mock et al. 2015, Energy conservation associated with ethanol formation from H₂ and CO₂ in Clostridium autoethanogenum involving electron bifurcation, J. Bacteriol. 197 (18) 2965; Nalakath, H. et al. 2015, Bioresource Technology 186, 122.). As described above, in organisms of the invention, it may be desired to target or knockout alcohol dehydrogenases responsible for ethanol production from the intermediate acetaldehyde to thereby promote synthesis of 3-hydroxybutanal from acetaldehyde catalysed by a DERA enzyme.

A further source of aldehyde ferredoxin oxidoreductase are the hyperthermophiles, Thermococcus sp. (Kesen, J. H. J. Bacteriol. 1995, 177, 4757 and Pyrococcus sp. (Basen et. al. 2014. PNAS, 111 (49), 17618 where this enzyme has been used to effectively synthesise ethanol from acetate via acetataldehyde driven by carbon monoxide. Although described mainly for oxidation of aldehydes to the corresponding acids, reduction of acetate is also mentioned. The Km values for acids appear higher than for the aldehydes, standard enzyme evolution techniques known in the art could be used to improve the enzyme's efficiency for acetate reduction. The use of aldehyde ferredoxin oxidoreductase in the aldehyde oxidation direction is further described by Kletzin, A., et al. J. Bacteriol. 1995, 177, 4817.

An aldehyde dehydrogenase (aldH) from E. coli has been shown to reduce 3-hydroxypropionic acid to the corresponding aldehyde as well as the preferred oxidation of 3-hydroxpropionaldehyde, Ji-Eun, J. et al., Appl. Microbiol. Biotechnol 2008. 81, 51. This enzyme was also shown to oxidise acetaldehyde to acetate. Hence, as these authors have shown the enzyme to be reversible, activity towards reduction of acetate would be expected.

TABLE 1
Examples of genes expressing enzymes for application to the reduction of
acetate to acetaldehyde (Activity A).

| UniProt | NCBI | EC | Gene | | |
|---|---|---|---|---|---|
| Entry | Gene ID | number | names | Protein names | Organism |
| P23883 | 12934534 | 1.2.1.5 | puuC | Aldehyde | Escherichia |
| | 947003 | | aldH | dehydrogenase PuuC | coli (strain |
| | | | b1300 | (EC 1.2.1.5) (3- | K12) |
| | | | JW1293 | hydroxypropionaldehyde | |
| | | | | dehydrogenase) | |
| | | | | (Gamma-glutamyl- | |
| | | | | gamma- | |
| | | | | aminobutyraldehyde | |
| | | | | dehydrogenase) | |
| | | | | (Gamma-Glu-gamma- | |
| | | | | aminobutyraldehyde | |
| | | | | dehydrogenase) | |
| D8GIZ8 | 9445627 | | CLJU_c20110 | Predicted tungsten- | Clostridium |
| | | | | containing aldehyde | ljungdahlii |
| | | | | ferredoxin | (strain ATCC |
| | | | | oxidoreductase | 55383/DSM |
| | | | | | 13528/ |
| | | | | | PETC) |
| D8GJ08 | 9445637 | | CLJU_c20210 | Predicted tungsten- | Clostridium |
| | | | | containing aldehyde | ljungdahlii |
| | | | | ferredoxin | (strain ATCC |
| | | | | oxidoreductase | 55383/DSM |
| | | | | | 13528/ |
| | | | | | PETC) |
| Q2RG52 | 3831332 | 1.2.7.5 | Moth_2300 | Aldehyde ferredoxin | Moorella |
| | | | | oxidoreductase (EC | thermoacetica |
| | | | | 1.2.7.5) | (strain ATCC |
| | | | | | 39073) |
| Q2RKJ9 | 3830998 | 1.2.7.5 | Moth_0722 | Aldehyde ferredoxin | Moorella |
| | | | | oxidoreductase (EC | thermoacetica |
| | | | | 1.2.7.5) | (strain ATCC |
| | | | | | 39073) |
| Q2RM47 | 3831866 | 1.2.7.5 | Moth_0154 | Aldehyde ferredoxin | Moorella |
| | | | | oxidoreductase (EC | thermoacetica |
| | | | | 1.2.7.5) | (strain ATCC |
| | | | | | 39073) |
| C9QU34 | 12705570; | 1.2.7.5 | ydhV | Aldehyde ferredoxin | Escherichia |
| | 12873031 | | EcDH1_1969 | oxidoreductase (EC | coli (strain |
| | | | ECDH1 | 1.2.7.5) (Putative | ATCC 33849/ |
| | | | ME8569_1617 | oxidoreductase) | DSM 4235/ |
| | | | | | NCIB 12045/ |
| | | | | | K12/DH1) |
| E8Y7H0 | 11776942; | 1.2.7.5 | ydhV | Aldehyde ferredoxin | Escherichia |
| | 12763367 | | EKO11_2102 | oxidoreductase (EC | coli (strain |
| | | | KO11_14380 | 1.2.7.5) (Putative | ATCC 55124/ |
| | | | | oxidoreductase) | KO11) |
| B1IQ83 | 6068384; | 1.2.7.5 | EcolC_1958 | Aldehyde ferredoxin | Escherichia |
| | | | | oxidoreductase (EC | coli (strain |
| | | | | 1.2.7.5) | ATCC 8739/ |
| | | | | | DSM 1576/ |
| | | | | | Crooks) |
| E0IXM3 | 12695599; | 1.2.7.5 | ydhV | Aldehyde ferredoxin | Escherichia |
| | 12753870 | | ECW_m1840 | oxidoreductase (EC | coli (strain |
| | | | WFL_09015 | 1.2.7.5) (Predicted | ATCC 9637/ |
| | | | EschWD | oxidoreductase) | CCM 2024/ |
| | | | RAFT_0881 | (Putative | DSM 1116/ |
| | | | | oxidoreductase) | NCIMB 8666/ |
| | | | | | NRRL B-766/ |
| | | | | | W) |
| C6EA37 | 8114754; | 1.2.7.5 | yagT | (2Fe—2S)-binding domain | Escherichia |
| | 8160069; | | B21_00248 | protein (Aldehyde | coli (strain B/ |
| | 8181416 | | ECBD_3371 | ferredoxin | BL21-DE3) |
| | | | ECD_00245 | oxidoreductase, Fe—S | |
| | | | | subunit, subunit of | |
| | | | | aldehyde ferredoxin | |
| | | | | oxidoreductase) (EC | |
| | | | | 1.2.7.5) (Predicted | |
| | | | | xanthine dehydrogenase, | |
| | | | | 2Fe—2S subunit) | |
| C6EA38 | 8114753; | 1.2.7.5 | yagS | Aldehyde ferredoxin | Escherichia |
| | 8160070; | | B21_00247 | oxidoreductase, FAD- | coli (strain B/ |
| | 8181415 | | ECBD_3372 | binding subunit, subunit | BL21-DE3) |
| | | | ECD_00244 | of aldehyde ferredoxin | |
| | | | | oxidoreductase (EC | |
| | | | | 1.2.7.5) (Molybdopterin | |
| | | | | dehydrogenase FAD- | |
| | | | | binding) (Predicted | |
| | | | | oxidoreductase with | |
| | | | | FAD-binding domain) | |
| C6EA39 | 8114752; | 1.2.7.5 | yagR | Aldehyde ferredoxin | Escherichia |
| | 8160071; | | B21_00246 | oxidoreductase: | coli (strain B/ |
| | 8181414 | | ECBD_3373 | molybdenum cofactor- | BL21-DE3) |
| | | | ECD_00243 | binding subunit, subunit | |
| | | | | of aldehyde ferredoxin | |
| | | | | oxidoreductase (EC | |
| | | | | 1.2.7.5) (Aldehyde | |
| | | | | oxidase and xanthine | |
| | | | | dehydrogenase | |
| | | | | molybdopterin binding) | |
| | | | | (Predicted | |
| | | | | oxidoreductase with | |
| | | | | molybdenum-binding | |
| | | | | domain) | |
| C6ECT2 | 8113808; | 1.2.7.5 | ydhV | Aldehyde ferredoxin | Escherichia |
| | 8157240; | | B21_01632 | oxidoreductase (EC | coli (strain B/ |
| | 8183188 | | ECBD_1972 | 1.2.7.5) (Predicted | BL21-DE3) |
| | | | ECD_01642 | oxidoreductase) | |
| K3JYI2 | | 1.2.7.5 | yagR | Aldehyde oxidase and | Escherichia |
| | | | EC3006_0366 | xanthine dehydrogenase | coli 3006 |
| | | | | (EC 1.2.7.5) | |
| A0A024KWW2 | | 1.2.7.5 | PGA_03435 | Aldehyde ferredoxin | Escherichia |
| | | | | oxidoreductase (EC | coli D6- |
| | | | | 1.2.7.5) | 113.11 |
| A0A024LHN1 | | 1.2.7.5 | PGC_20250 | Aldehyde ferredoxin | Escherichia |
| | | | | oxidoreductase (EC | coli D6- |
| | | | | 1.2.7.5) | 117.29 |
| Q56303 | 16548761 | 1.2.7.5 | for | Tungsten-containing | Thermococcus |
| | | | OCC_05029 | formaldehyde ferredoxin | litoralis |
| | | | | oxidoreductase (EC | (strain ATCC |
| | | | | 1.2.7.5) | 51850/DSM |
| | | | | | 5473/JCM |
| | | | | | 8560/NS-C) |
| Q6RKB1 | | 1.2.1.—; | car | Carboxylic acid | Nocardia |
| | | 1.2.1.30 | | reductase (CAR) (EC | iowensis |
| | | | | 1.2.1.—) (ATP/NADPH- | |
| | | | | dependent carboxylic | |
| | | | | acid reductase) (Aryl | |
| | | | | aldehyde | |
| | | | | oxidoreductase) (EC | |
| | | | | 1.2.1.30) | |
| A1YCA5 | | 2.7.8.7 | npt | 4′-phosphopantetheinyl | Nocardia |
| | | | | transferase Npt | iowensis |
| | | | | (PPTase) (EC 2.7.8.7) | |
| Q5YY80 | 3108003 | | NFA_20150 | Putative carboxylic acid | Nocardia |
| | | | | reductase | farcinica |
| | | | | | (strain IFM |
| | | | | | 10152) |
| Q5YSD9 | 3109498 | | NFA_40540 | Putative | Nocardia |
| | | | | phosphopantetheinyl | farcinica |
| | | | | transferase | (strain IFM |
| | | | | | 10152) |
| B1VMZ4 | 6209683 | | SGR_6790 | Putative carboxylic acid | Streptomyces |
| | | | | reductase | griseus |
| | | | | | subsp. |
| | | | | | griseus (strain |
| | | | | | JCM 4626/ |
| | | | | | NBRC 13350) |
| B1VRS6 | 6214265 | | SGR_665 | Putative | Streptomyces |
| | | | | phosphopantetheinyl | griseus |
| | | | | transferase | subsp. |
| | | | | | griseus (strain |
| | | | | | JCM 4626/ |
| | | | | | NBRC 13350) |
| B1VTI3 | 6210972; | | griD | Arylcarboxylate | Streptomyces |
| | | | SGR_4244 | reductase component | griseus |
| | | | | | subsp. |
| | | | | | griseus (strain |
| | | | | | JCM 4626/ |
| | | | | | NBRC 13350) |
| B1VTI2 | 6215140; | | griC | Arylcarboxylate | Streptomyces |
| | | | SGR_4243 | reductase component | griseus |
| | | | | | subsp. |
| | | | | | griseus (strain |
| | | | | | JCM 4626/ |
| | | | | | NBRC 13350) |
| Q51739 | 1468181 | 1.2.7.5 | aor, | Tungsten-containing | Pyrococcus |
| | | | AOR_PYRFU | aldehyde ferredoxin | furiosus |
| | | | | oxidoreductase | (strain ATCC |
| | | | | | 43587/DSM |
| | | | | | 3638/JCM |
| | | | | | 8422/Vc1) |

Additional car and npt genes and other genes coding for enzymes capable of (or involved with) carboxylic acid reduction (Activity A) can be identified based on sequence homology to those examples in Table 1.

### Route 2—Conversion of Acetyl CoA to Acetaldehyde Directly

Acetaldehyde can be synthesised from acetyl CoA via the reversible enzyme acetaldehyde dehydrogenase EC 1.2.1.10.

The gene coding for this enzyme can be found in a wide range of different organisms such as: Acinetobacter sp.; Burkholderia xenovorans; E. coli; Clostridium beijerinckii, (Run-Tao, Y and Jiann-Shin, C. 1990, Appl. Environ. Microbiol. 56, 2591; Appl. Environ Microbiol, 1999, 65 (11) 4973); Clostridium kluyveri; Pseudomonas sp. (Platt, A et al. 1995, Microbiol., 141, 2223; Soonyoung, H. et al. 1999, Biochem. Biophys. Res. Comm. 256, 469) Propionibacterium sp. and Thermoanaerobacter ethanolicus.

Many acetogens also have annotated acetaldehyde dehydrogenase genes e.g. Moorella thermoacetica (Moth_1776). Acetobacterium woodii (Arch. Microbiol, 1992, 158, 132). Clostridium ljungdahlii CLJU_c11960.

The eutE gene from the eut operon also encodes for an acetaldehyde dehydrogenase. The eutE gene from Salmonella enterica has been cloned into E. coli and shown to efficiently produce acetaldehyde from growth on glucose via acetyl CoA reduction (Huilin, Z. et al. 2011. Appl. Environ. Microbiol. 77, 6441). This is an excellent demonstration of an enzyme capable of efficiently providing acetaldehyde substrate for a DERA type enzyme catalysed aldol condensation in a 1,3-butanediol pathway from acetyl CoA. 1,3-Butanediol production using eutE to deliver acetaldehyde to DERA from acetyl CoA in a 1,3-BDO pathway, is shown in Example 10.

TABLE 2
Examples of genes expressing enzymes for the conversion of acetyl CoA to
acetaldehyde.(Activity B).

| UniProt | NCBI | EC | | | |
|---|---|---|---|---|---|
| Entry | Gene ID | number | Gene names | Protein names | Organism |
| H6LJM8 | 11871155 | 1.1.1.1; | adhE | Bifunctional | Acetobacterium |
| | | 1.2.1.10 | Awo_c06310 | acetaldehyde- | woodii (strain |
| | | | | CoA/alcohol | ATCC 29683/ |
| | | | | dehydrogenase | DSM 1030/JCM |
| | | | | (EC 1.1.1.1) (EC | 2381/KCTC |
| | | | | 1.2.1.10) | 1655) |
| Q79AF6 | 4010700 | 1.2.1.10; | bphJ | Acetaldehyde | Burkholderia |
| | | 1.2.1.87 | Bxeno_C1122 | dehydrogenase 4 | xenovorans |
| | | | Bxe_C1188 | (EC 1.2.1.10) | (strain LB400) |
| | | | | (Acetaldehyde | |
| | | | | dehydrogenase | |
| | | | | [acetylating] 4) | |
| | | | | (Propanal | |
| | | | | dehydrogenase | |
| | | | | (CoA- | |
| | | | | propanoylating)) | |
| | | | | (EC 1.2.1.87) | |
| Q143P8 | 4004910 | 1.2.1.10 | Bxeno_A0903 | Acetaldehyde | Burkholderia |
| | | | Bxe_A3547 | dehydrogenase 1 | xenovorans |
| | | | | (EC 1.2.1.10) | (strain LB400) |
| | | | | (Acetaldehyde | |
| | | | | dehydrogenase | |
| | | | | [acetylating] 1) | |
| Q13VU2 | 4002974 | 1.2.1.10 | amnH | Acetaldehyde | Burkholderia |
| | | | Bxeno_A3259 | dehydrogenase 2 | xenovorans |
| | | | Bxe_A1151 | (EC 1.2.1.10) | (strain LB400) |
| | | | | (Acetaldehyde | |
| | | | | dehydrogenase | |
| | | | | [acetylating] 2) | |
| Q13QH7 | 4007178 | 1.2.1.10 | Bxeno_B0694 | Acetaldehyde | Burkholderia |
| | | | Bxe_B2326 | dehydrogenase 3 | xenovorans |
| | | | | (EC 1.2.1.10) | (strain LB400) |
| | | | | (Acetaldehyde | |
| | | | | dehydrogenase | |
| | | | | [acetylating] 3) | |
| Q716S8 | 5294993 | 1.2.1.10 | ald | Aldehyde | Clostridium |
| | | | | dehydrogenase | beijerinckii |
| | | | | (EC 1.2.1.10) | (Clostridium MP) |
| | | | | (Coenzyme A | |
| | | | | acylating aldehyde | |
| | | | | dehydrogenase) | |
| | | | | (EC 1.2.1.10) | |
| | | | | (Coenzyme A- | |
| | | | | acylating aldehyde | |
| | | | | dehydrogenase) | |
| | | | | (EC 1.2.1.10) | |
| D8GIC3 | 9444813; | 1.2.1.— | CLJU_c11960 | Predicted | Clostridium |
| | 9447589 | | CLJU_c39730 | acetaldehyde | ljungdahlii (strain |
| | | | | dehydrogenase | ATCC 55383/ |
| | | | | (EC 1.2.1.—) | DSM 13528/ |
| | | | | | PETC) |
| D8GID4 | 9447600 | 1.2.1.— | CLJU_c39840 | Predicted | Clostridium |
| | | | | acetaldehyde | ljungdahlii (strain |
| | | | | dehydrogenase | ATCC 55383/ |
| | | | | (EC 1.2.1.—) | DSM 13528/ |
| | | | | | PETC) |
| P77580 | 12932628; | 1.2.1.10 | mhpF mhpE | Acetaldehyde | Escherichia coli |
| | 945008 | | b0351 | dehydrogenase | (strain K12) |
| | | | JW0342 | (EC 1.2.1.10) | |
| | | | | (Acetaldehyde | |
| | | | | dehydrogenase | |
| | | | | [acetylating]) | |
| A4IT43 | 4968078 | 1.2.1.10 | nbaJ | Acetaldehyde | Geobacillus |
| | | | GTNG_3152 | dehydrogenase | thermodenitrificans |
| | | | | (EC 1.2.1.10) | (strain NG80- |
| | | | | (Acetaldehyde | 2) |
| | | | | dehydrogenase | |
| | | | | [acetylating]) | |
| Q2RHL2 | 3832442 | 1.2.1.10 | Moth_1776 | Acetaldehyde | Moorella |
| | | | | dehydrogenase | thermoacetica |
| | | | | (EC 1.2.1.10) | (strain ATCC |
| | | | | (Acetaldehyde | 39073) |
| | | | | dehydrogenase | |
| | | | | [acetylating]) | |
| C8CEC3 | | 1.2.1.10 | nahO | Acetaldehyde | Pseudomonas |
| | | | | dehydrogenase | aeruginosa |
| | | | | (EC 1.2.1.10) | |
| | | | | (Acetaldehyde | |
| | | | | dehydrogenase | |
| | | | | [acetylating]) | |
| P41793 | 1253985 | | eutE | Ethanolamine | Salmonella |
| | | | STM2463 | utilization protein | typhimurium |
| | | | | EutE | (strain LT2/ |
| | | | | | SGSC1412/ |
| | | | | | ATCC 700720) |

Additional genes coding for enzymes capable of acetyl CoA conversion to acetaldehyde (Activity B) can be identified based on sequence homology to those examples in Table 2

### Route 3. Conversion of Pyruvate to Acetaldehyde Via Acetyl CoA

The conversion of pyruvate to acetyl CoA can be carried out using an enzyme such as EC 1.2.7.1 (pyruvate synthase, pyruvate:ferredoxin oxidoreductase). These ferredoxin linked enzymes are particularly common in anaerobes such as the acetogens, but are also present in other aerobic or facultatively anaerobic organisms such as Hydrogenobacter thermophilus.

The pyruvate dehydrogenase complex is also a central metabolic enzyme well understood in the art which is responsible for conversion of pyruvate (for example, generated from glycolysis) to acetyl CoA for entry into the TCA cycle.

The subsequent conversion of acetyl CoA to acetaldehyde is described in Route 2.

TABLE 3
Examples of genes expressing enzymes for the conversion of pyruvate to acetyl
CoA (Activity C).

| UniProt | NCBI Gene | | | |
|---|---|---|---|---|
| Entry | ID | Gene names | Protein names | Organism |
| H6LJ55 | 11873437 | porB | Pyruvate:ferredoxin | Acetobacterium |
| | | Awo_c06200 | oxidoreductase, beta | woodii (strain ATCC |
| | | | subunit PorB (EC | 29683/DSM 1030/ |
| | | | 1.2.7.1) | JCM 2381/KCTC |
| | | | | 1655) |
| H6LJ56 | 11873438 | porA | Pyruvate:ferredoxin | Acetobacterium |
| | | Awo_c06210 | oxidoreductase, alpha | woodii (strain ATCC |
| | | | subunit PorA (EC | 29683/DSM 1030/ |
| | | | 1.2.7.1) | JCM 2381/KCTC |
| | | | | 1655) |
| U1VQ53 | | BTCBT_005517 | Pyruvate synthase | Bacillus |
| | | | subunit porA (EC | thuringiensis T01- |
| | | | 1.2.7.1) | 328 |
| U1W7V0 | | BTCBT_005516 | Pyruvate synthase | Bacillus |
| | | | subunit porB (EC | thuringiensis T01- |
| | | | 1.2.7.1) | 328 |
| E5ZLB4 | | nifJ | Pyruvate synthase | Campylobacter |
| | | CSU_1742 | (EC 1.2.7.1) | jejuni subsp. jejuni |
| | | | | 327 |
| A5I7E8 | 5187682; | CBO3423 | Putative subunit of | Clostridium |
| | 5400580 | CLC_3367 | pyruvate:flavodoxin | botulinum (strain |
| | | | oxidoreductase (EC | Hall/ATCC 3502/ |
| | | | 1.2.7.1) | NCTC 13319/Type |
| | | | | A) |
| A5I7E6 | 16691482; | CBO3421 | Putative subunit of | Clostridium |
| | 5186833 | | pyruvate:flavodoxin | botulinum (strain |
| | | | oxidoreductase (EC | Hall/ATCC 3502/ |
| | | | 1.2.7.1) | NCTC 13319/Type |
| | | | | A) |
| A5N1K8 | 5390957 | porB | PorB (EC 1.2.7.1) | Clostridium kluyveri |
| | | CKL_2996 | | (strain ATCC 8527/ |
| | | | | DSM 555/NCIMB |
| | | | | 10680) |
| A5N1L1 | 5393792 | porC | PorC (EC 1.2.7.1) | Clostridium kluyveri |
| | | CKL_2999 | | (strain ATCC 8527/ |
| | | | | DSM 555/NCIMB |
| | | | | 10680) |
| A5N1K9 | 5390958 | porA | PorA (EC 1.2.7.1) | Clostridium kluyveri |
| | | CKL_2997 | | (strain ATCC 8527/ |
| | | | | DSM 555/NCIMB |
| | | | | 10680) |
| A5N1L0 | 5393791 | porD | PorD (EC 1.2.7.1) | Clostridium kluyveri |
| | | CKL_2998 | | (strain ATCC 8527/ |
| | | | | DSM 555/NCIMB |
| | | | | 10680) |
| Q9LBG1 | 12100419; | PorE | Pyruvate:ferredoxin | Hydrogenobacter |
| | 8773721 | | oxidoreductase | thermophilus |
| | | | epsilon subunit (EC | |
| | | | 1.2.7.1) | |
| Q9LBF7 | 12100415; | porG | Pyruvate:ferredoxin | Hydrogenobacter |
| | 8773666 | | oxidoreductase | thermophilus |
| | | | gamma subunit (EC | |
| | | | 1.2.7.1) | |
| Q9LBF8 | 12100416; | porB | Pyruvate:ferredoxin | Hydrogenobacter |
| | 8773723 | | oxidoreductase beta | thermophilus |
| | | | subunit (EC 1.2.7.1) | |
| Q9LBF9 | 12100417; | porA | Pyruvate:ferredoxin | Hydrogenobacter |
| | 8773720 | | oxidoreductase alpha | thermophilus |
| | | | subunit (EC 1.2.7.1) | |
| Q9LBG0 | 12100418; | porD | Pyruvate:ferredoxin | Hydrogenobacter |
| | 8773719 | | oxidoreductase delta | thermophilus |
| | | | subunit (EC 1.2.7.1) | |
| P80900 | 9704020 | porA | Pyruvate synthase | Methanothermobacter |
| | | MTBMA_c03140 | subunit PorA (EC | marburgensis |
| | | | 1.2.7.1) (Pyruvate | (strain DSM 2133/ |
| | | | oxidoreductase alpha | 14651/NBRC |
| | | | chain) (POR) | 100331/OCM 82/ |
| | | | (Pyruvic-ferredoxin | Marburg) |
| | | | oxidoreductase | (Methanobacterium |
| | | | subunit alpha) | thermoautotrophicum) |
| P80901 | 9704019 | porB | Pyruvate synthase | Methanothermobacter |
| | | MTBMA_c03130 | subunit PorB (EC | marburgensis |
| | | | 1.2.7.1) (Pyruvate | (strain DSM 2133/ |
| | | | oxidoreductase beta | 14651/NBRC |
| | | | chain) (POR) | 100331/OCM 82/ |
| | | | (Pyruvic-ferredoxin | Marburg) |
| | | | oxidoreductase | (Methanobacterium |
| | | | subunit beta) | thermoautotrophicum) |
| P80902 | 9704022 | porC | Pyruvate synthase | Methanothermobacter |
| | | MTBMA_c03160 | subunit PorC (EC | marburgensis |
| | | | 1.2.7.1) (Pyruvate | (strain DSM 2133/ |
| | | | oxidoreductase | 14651/NBRC |
| | | | gamma chain) (POR) | 100331/OCM 82/ |
| | | | (Pyruvic-ferredoxin | Marburg) |
| | | | oxidoreductase | (Methanobacterium |
| | | | subunit gamma) | thermoautotrophicum) |
| Q2RH65 | 3830848 | Moth_1924 | Pyruvate ferredoxin | Moorella |
| | | | oxidoreductase, | thermoacetica |
| | | | gamma subunit (EC | (strain ATCC |
| | | | 1.2.7.1) | 39073) |
| Q2RLH9 | 3832620 | Moth_0376 | Pyruvate ferredoxin | Moorella |
| | | | oxidoreductase, | thermoacetica |
| | | | gamma subunit (EC | (strain ATCC |
| | | | 1.2.7.1) | 39073) |
| Q2RI42 | 3832737 | Moth_1591 | Pyruvate ferredoxin | Moorella |
| | | | oxidoreductase, beta | thermoacetica |
| | | | subunit (EC 1.2.7.1) | (strain ATCC |
| | | | | 39073) |
| Q2RH67 | 3830846 | Moth_1922 | Pyruvate ferredoxin | Moorella |
| | | | oxidoreductase, alpha | thermoacetica |
| | | | subunit (EC 1.2.7.1) | (strain ATCC |
| | | | | 39073) |
| Q2RLH7 | 3832622 | Moth_0378 | Pyruvate ferredoxin | MooreIla |
| | | | oxidoreductase, alpha | thermoacetica |
| | | | subunit (EC 1.2.7.1) | (strain ATCC |
| | | | | 39073) |
| Q2RH68 | 3830845 | Moth_1921 | Pyruvate ferredoxin | MooreIla |
| | | | oxidoreductase, beta | thermoacetica |
| | | | subunit (EC 1.2.7.1) | (strain ATCC |
| | | | | 39073) |
| Q51804 | 1468831 | porA PF0966 | Pyruvate synthase | Pyrococcus furiosus |
| | | | subunit PorA (EC | (strain ATCC 43587/ |
| | | | 1.2.7.1) (Pyruvate | DSM 3638/JCM |
| | | | oxidoreductase alpha | 8422/Vc1) |
| | | | chain) (POR) | |
| | | | (Pyruvic-ferredoxin | |
| | | | oxidoreductase | |
| | | | subunit alpha) | |
| Q51805 | 1468830 | porB PF0965 | Pyruvate synthase | Pyrococcus furiosus |
| | | | subunit PorB (EC | (strain ATCC 43587/ |
| | | | 1.2.7.1) (Pyruvate | DSM 3638/JCM |
| | | | oxidoreductase beta | 8422/Vc1) |
| | | | chain) (POR) | |
| | | | (Pyruvic-ferredoxin | |
| | | | oxidoreductase | |
| | | | subunit beta) | |
| O05651 | 896831 | porA TM_0017 | Pyruvate synthase | Thermotoga |
| | | | subunit PorA (EC | maritima (strain |
| | | | 1.2.7.1) (Pyruvate | ATCC 43589/ |
| | | | oxidoreductase alpha | MSB8/DSM 3109/ |
| | | | chain) (POR) | JCM 10099) |
| | | | (Pyruvic-ferredoxin | |
| | | | oxidoreductase | |
| | | | subunit alpha) | |
| O05650 | 896829 | porC porG | Pyruvate synthase | Thermotoga |
| | | TM_0015 | subunit PorC (EC | maritima (strain |
| | | | 1.2.7.1) (Pyruvate | ATCC 43589/ |
| | | | oxidoreductase | MSB8/DSM 3109/ |
| | | | gamma chain) (POR) | JCM 10099) |
| | | | (Pyruvic-ferredoxin | |
| | | | oxidoreductase | |
| | | | subunit gamma) | |
| Q56317 | 896832 | porB TM_0018 | Pyruvate synthase | Thermotoga |
| | | | subunit PorB (EC | maritima (strain |
| | | | 1.2.7.1) (Pyruvate | ATCC 43589/ |
| | | | oxidoreductase beta | MSB8/DSM 3109/ |
| | | | chain) (POR) | JCM 10099) |
| | | | (Pyruvic-ferredoxin | |
| | | | oxidoreductase | |
| | | | subunit beta) | |

Additional genes coding for enzymes capable of the conversion of pyruvate to acetyl CoA (Activity C) can be identified based on sequence homology to those examples in Table 3, or to common sequences for the pyruvate dehydrogenase complex.

### Route 4. Conversion of Pyruvate to Acetaldehyde Directly

The conversion of pyruvate to acetaldehyde is well known in the art. Pyruvate decarboxylase is a homotetrameric enzyme (EC 4.1.1.1) that catalyses the decarboxylation of pyruvic acid to acetaldehyde and carbon dioxide in the cytoplasm of prokaryotes, and in the mitochondria of eukaryotes. It is also called 2-oxo-acid carboxylase, alpha-ketoacid carboxylase, and pyruvic decarboxylase. Under anaerobic conditions, this enzyme is part of the fermentation process that occurs in yeast, especially of the Saccharomyces genus, to produce ethanol by fermentation. Pyruvate decarboxylase starts this process by converting pyruvate into acetaldehyde and carbon dioxide.

The pyruvate ferredoxin oxidoreductase from Pyrococcus furiosus (Table 3) has also been shown to catalyse pyruvate decarboxylation to acetaldehyde (Ma, K. et al. 1997. PNAS, 94, 9608).

Examples 12,13 and 14 show the production of 1,3-butanediol using pyruvate decarboxylase to deliver acetaldehyde to DERA from pyruvate, in a novel, unnatural 1,3-BDO pathway. Table 4. Examples of genes expressing enzymes for application to the decarboxylation of pyruvate to acetaldehyde (Activity D).

| Uniprot | NCBI | | | |
|---|---|---|---|---|
| Entry | GeneID | Gene names | Protein names | Organism |
| O82647 | 829444 | PDC1 At4g33070 | Pyruvate | Arabidopsis thaliana |
| | | F4I10.4 | decarboxylase 1 | (Mouse-ear cress) |
| | | | (AtPDC1) | |
| Q9FFT4 | 835587 | PDC2 At5g54960 | Pyruvate | Arabidopsis thaliana |
| | | MBG8.23 | decarboxylase 2 | (Mouse-ear cress) |
| | | | (AtPDC2) | |
| Q9M039 | 831414 | PDC3 At5g01330 | Pyruvate | Arabidopsis thaliana |
| | | T10O8.40 | decarboxylase 3 | (Mouse-ear cress) |
| | | | (AtPDC3) | |
| Q9M040 | 830867 | PDC4 At5g01320 | Pyruvate | Arabidopsis thaliana |
| | | T10O8.30 | decarboxylase 4 | (Mouse-ear cress) |
| | | | (AtPDC4) | |
| Q2UKV4 | 5991796 | pdcA | Pyruvate | Aspergillus oryzae (strain |
| | | AO090003000661 | decarboxylase | ATCC 42149/RIB 40) |
| | | | | (Yellow koji mold) |
| P51844 | | pdcA pdc | Pyruvate | Aspergillus parasiticus |
| | | | decarboxylase | |
| Q0CNV1 | 4320296 | pdcA ATEG_04633 | Pyruvate | Aspergillus terreus (strain |
| | | | decarboxylase | NIH 2624/FGSC A1156) |
| P83779 | 3642780 | PDC11 PDC1 | Pyruvate | Candida albicans (strain |
| | | CaO19.10395 | decarboxylase | SC5314/ATCC MYA- |
| | | CaO19.2877 | | 2876) (Yeast) |
| Q6FJA3 | 2891742 | PDC1 PDC | Pyruvate | Candida glabrata (strain |
| | | CAGL0M07920g | decarboxylase | ATCC 2001/CBS 138/ |
| | | | | JCM 3761/NBRC 0622/ |
| | | | | NRRL Y-65) (Yeast) |
| | | | | (Torulopsis glabrata) |
| P87208 | 2872690 | pdcA AN4888 | Pyruvate | Emericella nidulans |
| | | | decarboxylase | (strain FGSC A4/ATCC |
| | | | | 38163/CBS 112.46/ |
| | | | | NRRL 194/M139) |
| | | | | (Aspergillus nidulans) |
| P34734 | | PDC | Pyruvate | Hanseniaspora uvarum |
| | | | decarboxylase | (Yeast) (Kloeckera |
| | | | | apiculata) |
| Q12629 | 2894295 | PDC1 | Pyruvate | Kluyveromyces lactis |
| | | KLLA0E16357g | decarboxylase | (strain ATCC 8585/CBS |
| | | | | 2359/DSM 70799/ |
| | | | | NBRC 1267/NRRL Y- |
| | | | | 1140/WM37) (Yeast) |
| | | | | (Candida sphaerica) |
| P33149 | | PDC1 | Pyruvate | Kluyveromyces |
| | | | decarboxylase | marxianus (Yeast) |
| | | | | (Candida kefyr) |
| Q4WXX9 | 3511715 | pdcA | Pyruvate | Neosartorya fumigata |
| | | AFUA_3G11070 | decarboxylase | (strain ATCC MYA-4609/ |
| | | | | Af293/CBS 101355/ |
| | | | | FGSC A1100) |
| | | | | (Aspergillus fumigatus) |
| P33287 | 3875734 | cfp pdc-1 | Pyruvate | Neurospora crassa (strain |
| | | NCU02193 | decarboxylase | ATCC 24698/74-OR23- |
| | | | (8-10 nm | 1A/CBS 708.71/DSM |
| | | | cytoplasmic | 1257/FGSC 987) |
| | | | filament- | |
| | | | associated | |
| | | | protein) (P59NC) | |
| A2Y5L9 | | PDC1 OsI_019612 | Pyruvate | Oryza sativa subsp. |
| | | | decarboxylase 1 | indica (Rice) |
| | | | (PDC) | |
| A2XFI3 | | PDC2 OsI_010826 | Pyruvate | Oryza sativa subsp. |
| | | | decarboxylase 2 | indica (Rice) |
| | | | (PDC) | |
| A2YQ76 | | PDC3 OsI_026469 | Pyruvate | Oryza sativa subsp. |
| | | | decarboxylase 3 | indica (Rice) |
| | | | (PDC) | |
| Q0DHF6 | 4339066 | PDC1 | Pyruvate | Oryza sativa subsp. |
| | | Os05g0469600 | decarboxylase 1 | japonica (Rice) |
| | | LOC_Os05g39310 | (PDC) | |
| | | OsJ_018109 | | |
| | | OSJNBa0052E20.2 | | |
| Q10MW3 | 4332519 | PDC2 | Pyruvate | Oryza sativa subsp. |
| | | Os03g0293500 | decarboxylase 2 | japonica (Rice) |
| | | LOC_Os03g18220 | (PDC) | |
| Q0D3D2 | 4344382 | PDC3 | Pyruvate | Oryza sativa subsp. |
| | | Os07g0693100 | decarboxylase 3 | japonica (Rice) |
| | | LOC_Os07g49250 | (PDC) | |
| | | OsJ_024667 | | |
| P51850 | | PDC1 | Pyruvate | Pisum sativum (Garden |
| | | | decarboxylase 1 | pea) |
| | | | (PDC) | |
| P06169 | 850733 | PDC1 YLR044C | Pyruvate | Saccharomyces |
| | | L2104 | decarboxylase | cerevisiae (strain ATCC |
| | | | isozyme 1 (EC | 204508/S288c) (Baker's |
| | | | 4.1.1.—) | yeast) |
| P16467 | 850825 | PDC5 YLR134W | Pyruvate | Saccharomyces |
| | | L3133 L9606.7 | decarboxylase | cerevisiae (strain ATCC |
| | | | isozyme 2 (EC | 204508/S288c) (Baker's |
| | | | 4.1.1.—) | yeast) |
| P26263 | 852978 | PDC6 YGR087C | Pyruvate | Saccharomyces |
| | | | decarboxylase | cerevisiae (strain ATCC |
| | | | isozyme 3 | 204508/S288c) (Baker's |
| | | | | yeast) |
| O42873 | 2543400 | SPAC3G9.11c | Putative pyruvate | Schizosaccharomyces |
| | | | decarboxylase | pombe (strain 972/ |
| | | | C3G9.11c | ATCC 24843) (Fission |
| | | | | yeast) |
| Q09737 | 3361478 | SPAC13A11.06 | Putative pyruvate | Schizosaccharomyces |
| | | SPAC3H8.01 | decarboxylase | pombe (strain 972/ |
| | | | C13A11.06 | ATCC 24843) (Fission |
| | | | | yeast) |
| Q9P7P6 | 2542602 | SPAC186.09 | Probable | Schizosaccharomyces |
| | | | pyruvate | pombe (strain 972/ |
| | | | decarboxylase | ATCC 24843) (Fission |
| | | | C186.09 | yeast) |
| P28516 | 542376 | PDC1 PDC | Pyruvate | Zea mays (Maize) |
| | | | decarboxylase 1 | |
| | | | (PDC) | |
| P06672 | 3188496 | pdc ZMO1360 | Pyruvate | Zymomonas mobilis |
| | | | decarboxylase | subsp. mobilis (strain |
| | | | (PDC) | ATCC 31821/ZM4/ |
| | | | | CP4) |

Additional genes coding for enzymes capable of pyruvate conversion to acetaldehyde (Activity D) can be identified based on sequence homology to those examples in Table 4.

### Route 5. Conversion of Acetyl CoA to Acetaldehyde Via Pyruvate

The conversion of acetyl CoA to pyruvate (Activity E) can be achieved using the reversible enzyme pyruvate ferredoxin oxidoreductase (pyruvate synthase, EC 1.2.7.1). Gene sequences coding for this enzyme are listed in Table 3.

The subsequent conversion of pyruvate to acetaldehyde is described in Route 4.

### Route 6. Conversion of Acetate to Acetaldehyde Via Acetyl CoA.

The conversion of acetate to acetyl CoA can be achieved using acetyl CoA synthetase or a CoA transferase for example, EC 6.2.1.1 or EC 2.8.3.8 and subsequently converted to acetaldehyde via EC 1.2.1.10 (Route 2.). Examples of gene sequences coding for enzymes capable of the conversion of acetate to acetyl CoA are shown in Table 5.

TABLE 5
Examples of genes expressing enzymes for application to the synthesis of acetyl
CoA from acetate (Activity F).

| UniProt | NCBI | Gene | | |
|---|---|---|---|---|
| Entry | Gene ID | names | Protein names | Organism |
| A4SJM6 | 4995560 | acsA | Acetyl-coenzyme A | Aeromonas |
| | | ASA_0967 | synthetase (AcCoA | salmonicida |
| | | | synthetase) (Acs) (EC | (strain A449) |
| | | | 6.2.1.1) (Acetate--CoA | |
| | | | ligase) (Acyl-activating | |
| | | | enzyme) | |
| Q9KWA3 | 874783 | acsA | Acetyl-coenzyme A | Agrobacterium |
| | | riorf81 | synthetase (AcCoA | rhizogenes |
| | | | synthetase) (Acs) (EC | |
| | | | 6.2.1.1) (Acetate--CoA | |
| | | | ligase) (Acyl-activating | |
| | | | enzyme) | |
| Q8UBV5 | 1134783 | acsA acs | Acetyl-coenzyme A | Agrobacterium |
| | | Atu2745 | synthetase (AcCoA | tumefaciens |
| | | AGR_C_4980 | synthetase) (Acs) (EC | (strain C58/ |
| | | | 6.2.1.1) (Acetate--CoA | ATCC 33970) |
| | | | ligase) (Acyl-activating | |
| | | | enzyme) | |
| Q758X0 | 4620668 | ACS1 | Acetyl-coenzyme A | Ashbya |
| | | ADR408W | synthetase 1 (EC 6.2.1.1) | gossypii (strain |
| | | | (Acetate--CoA ligase 1) | ATCC 10895/ |
| | | | (Acyl-activating enzyme | CBS 109.51/ |
| | | | 1) | FGSC 9923/ |
| | | | | NRRL Y-1056) |
| | | | | (Yeast) |
| | | | | (Eremothecium |
| | | | | gossypii) |
| Q750T7 | 4622812 | ACS2 | Acetyl-coenzyme A | Ashbya |
| | | AGL148C | synthetase 2 (EC 6.2.1.1) | gossypii (strain |
| | | | (Acetate--CoA ligase 2) | ATCC 10895/ |
| | | | (Acyl-activating enzyme | CBS 109.51/ |
| | | | 2) | FGSC 9923/ |
| | | | | NRRL Y-1056) |
| | | | | (Yeast) |
| | | | | (Eremothecium |
| | | | | gossypii) |
| P39062 | 937324 | acsA | Acetyl-coenzyme A | Bacillus |
| | | BSU29680 | synthetase (AcCoA | subtilis (strain |
| | | | synthetase) (Acs) (EC | 168) |
| | | | 6.2.1.1) (Acetate--CoA | |
| | | | ligase) (Acyl-activating | |
| | | | enzyme) | |
| Q89WV5 | 1049589 | acsA | Acetyl-coenzyme A | Bradyrhizobium |
| | | blr0573 | synthetase (AcCoA | diazoefficiens |
| | | | synthetase) (Acs) (EC | (strain JCM |
| | | | 6.2.1.1) (Acetate--CoA | 10833/IAM |
| | | | ligase) (Acyl-activating | 13628/NBRC |
| | | | enzyme) | 14792/USDA |
| | | | | 110) |
| Q8FYQ3 | 1167504; | acsA | Acetyl-coenzyme A | Brucella suis |
| | 12137575 | BR1811 | synthetase (AcCoA | biovar 1 (strain |
| | | BS1330_I1805 | synthetase) (Acs) (EC | 1330) |
| | | | 6.2.1.1) (Acetate--CoA | |
| | | | ligase) (Acyl-activating | |
| | | | enzyme) | |
| Q8NJN3 | 3644652; | ACS2 | Acetyl-coenzyme A | Candida |
| | 3644710 | CaO19.1064 | synthetase 2 (EC 6.2.1.1) | albicans (strain |
| | | CaO19.8666 | (Acetate--CoA ligase 2) | SC5314/ |
| | | | (Acyl-activating enzyme | ATCC MYA- |
| | | | 2) | 2876) (Yeast) |
| Q8KBY0 | 1006138 | acsA acs | Acetyl-coenzyme A | Chlorobium |
| | | CT1652 | synthetase (AcCoA | tepidum (strain |
| | | | synthetase) (Acs) (EC | ATCC 49652/ |
| | | | 6.2.1.1) (Acetate--CoA | DSM 12025/ |
| | | | ligase) (Acyl-activating | TLS) |
| | | | enzyme) | |
| P16928 | 2871910 | facA | Acetyl-coenzyme A | Emericella |
| | | acuA | synthetase (EC 6.2.1.1) | nidulans |
| | | AN5626 | (Acetate--CoA ligase) | (strain FGSC |
| | | | (Acyl-activating enzyme) | A4/ATCC |
| | | | | 38163/CBS |
| | | | | 112.46/NRRL |
| | | | | 194/M139) |
| | | | | (Aspergillus |
| | | | | nidulans) |
| P27550 | 12933681; | acs yfaC | Acetyl-coenzyme A | Escherichia |
| | 948572 | b4069 | synthetase (AcCoA | coli (strain |
| | | JW4030 | synthetase) (Acs) (EC | K12) |
| | | | 6.2.1.1) (Acetate--CoA | |
| | | | ligase) (Acyl-activating | |
| | | | enzyme) | |
| O60011 | 2896335 | ACS1 | Acetyl-coenzyme A | Kluyveromyces |
| | | KLLA0A03333g | synthetase 1 (EC 6.2.1.1) | lactis (strain |
| | | | (Acetate--CoA ligase 1) | ATCC 8585/ |
| | | | (Acyl-activating enzyme | CBS 2359/ |
| | | | 1) | DSM 70799/ |
| | | | | NBRC 1267/ |
| | | | | NRRL Y-1140/ |
| | | | | WM37) |
| | | | | (Yeast) |
| | | | | (Candida |
| | | | | sphaerica) |
| P27095 | | acsA acs | Acetyl-coenzyme A | Methanosaeta |
| | | | synthetase (AcCoA | concilii |
| | | | synthetase) (Acs) (EC | (Methanothrix |
| | | | 6.2.1.1) (Acetate--CoA | soehngenii) |
| | | | ligase) (Acyl-activating | |
| | | | enzyme) | |
| P9WQD1 | 13317276; | acsA acs | Acetyl-coenzyme A | Mycobacterium |
| | 885479 | Rv3667 | synthetase (AcCoA | tuberculosis |
| | | MTV025.015 | synthetase) (Acs) (EC | (strain ATCC |
| | | | 6.2.1.1) (Acetate--CoA | 25618/ |
| | | | ligase) (Acyl-activating | H37Rv) |
| | | | enzyme) | |
| O93730 | 1463659 | acsA acs | Acetyl-coenzyme A | Pyrobaculum |
| | | PAE2867 | synthetase (AcCoA | aerophilum |
| | | | synthetase) (Acs) (EC | (strain ATCC |
| | | | 6.2.1.1) (Acetate--CoA | 51768/IM2/ |
| | | | ligase) (Acyl-activating | DSM 7523/ |
| | | | enzyme) | JCM 9630/ |
| | | | | NBRC |
| | | | | 100827) |
| Q9Z3R3 | 1232358 | acsA1 | Acetyl-coenzyme A | Rhizobium |
| | | R00719 | synthetase 1 (AcCoA | meliloti (strain |
| | | SMc00774 | synthetase 1) (Acs 1) (EC | 1021) (Ensifer |
| | | | 6.2.1.1) (Acetate--CoA | meliloti) |
| | | | ligase 1) (Acyl-activating | (Sinorhizobium |
| | | | enzyme 1) | meliloti) |
| O68040 | 9004945 | acsA acs | Acetyl-coenzyme A | Rhodobacter |
| | | RCAP_rc | synthetase (AcCoA | capsulatus |
| | | c02126 | synthetase) (Acs) (EC | (strain ATCC |
| | | | 6.2.1.1) (Acetate--CoA | BAA-309/ |
| | | | ligase) (Acyl-activating | NBRC 16581/ |
| | | | enzyme) | SB1003) |
| Q01574 | 851245 | ACS1 | Acetyl-coenzyme A | Saccharomyces |
| | | YAL054C | synthetase 1 (EC 6.2.1.1) | cerevisiae |
| | | FUN44 | (Acetate--CoA ligase 1) | (strain ATCC |
| | | | (Acyl-activating enzyme | 204508/ |
| | | | 1) | S288c) |
| | | | | (Baker's yeast) |
| Q8ZKF6 | 1255801 | acs | Acetyl-coenzyme A | Salmonella |
| | | STM4275 | synthetase (AcCoA | typhimurium |
| | | | synthetase) (Acs) (EC | (strain LT2/ |
| | | | 6.2.1.1) (Acetate--CoA | SGSC1412/ |
| | | | ligase) (Acyl-activating | ATCC 700720) |
| | | | enzyme) | |
| Q82EL5 | 1211019 | acsA | Acetyl-coenzyme A | Streptomyces |
| | | SAV_4599 | synthetase (AcCoA | avermitilis |
| | | | synthetase) (Acs) (EC | (strain ATCC |
| | | | 6.2.1.1) (Acetate--CoA | 31267/DSM |
| | | | ligase) (Acyl-activating | 46492/JCM |
| | | | enzyme) | 5070/NCIMB |
| | | | | 12804/NRRL |
| | | | | 8165/MA- |
| | | | | 4680) |
| Q55404 | 951871 | acsA acs | Acetyl-coenzyme A | Synechocystis |
| | | sll0542 | synthetase (AcCoA | sp. (strain |
| | | | synthetase) (Acs) (EC | PCC 6803/ |
| | | | 6.2.1.1) (Acetate--CoA | Kazusa) |
| | | | ligase) (Acyl-activating | |
| | | | enzyme) | |
| F1CYZ5 | | carA | Acetate CoA-transferase | Acetobacterium |
| | | | YdiF (EC 2.8.3.8) | woodii |
| H6LGM4 | 11871862 | carA2 | Acetate CoA-transferase | Acetobacterium |
| | | Awo_c15700 | YdiF (EC 2.8.3.8) | woodii |
| | | | | (strain ATCC |
| | | | | 29683/DSM |
| | | | | 1030/JCM |
| | | | | 2381/KCTC |
| | | | | 1655) |
| G6XSJ6 | | ATCR1_08124 | Acetate CoA-transferase | Agrobacterium |
| | | | YdiF (EC 2.8.3.8) | tumefaciens |
| | | | | CCNWGS0286 |
| V5MSC1 | 17703598 | U712_10415 | Putative coenzyme A | Bacillus |
| | | | transferase subunit beta | subtilis PY79 |
| | | | (EC 2.8.3.8) | |
| V5MSQ3 | 17703599 | U712_10420 | Putative coenzyme A | Bacillus |
| | | | transferase subunit alpha | subtilis PY79 |
| | | | (EC 2.8.3.8) | |
| Q8FY42 | 1167750; | BR2047 | Acetate CoA-transferase | Brucella suis |
| | 12137830 | BS1330_I2041 | YdiF (EC 2.8.3.8) | biovar 1 (strain |
| | | | | 1330) |
| C6Q271 | | Ccar_0559 | Acetate CoA-transferase | Clostridium |
| | | CcarbDRAFT_5139 | YdiF (EC 2.8.3.8) | carboxidivorans |
| | | CLCAR_0656 | | P7 |
| P76458 | 12930185; | atoD | Acetate CoA-transferase | Escherichia |
| | 947525 | b2221 | subunit alpha (EC 2.8.3.8) | coli (strain |
| | | JW2215 | (Acetyl- | K12) |
| | | | CoA:acetoacetate-CoA | |
| | | | transferase subunit alpha) | |
| P76459 | 12933993; | atoA | Acetate CoA-transferase | Escherichia |
| | 946719 | b2222 | subunit beta (EC 2.8.3.8) | coli (strain |
| | | JW2216 | (Acetyl-CoA:acetoacetate | K12) |
| | | | CoA-transferase subunit | |
| | | | beta) | |
| P37766 | 12931296; | ydiF | Acetate CoA-transferase | Escherichia |
| | 946211 | b1694 | YdiF (EC 2.8.3.8) (Short- | coli (strain |
| | | JW1684 | chain acyl-CoA:acetate | K12) |
| | | | CoA-transferase) | |
| A9HGB7 | 5790057; | GDI1530 | Acetate CoA-transferase | Gluconacetobacter |
| | 6975653 | Gdia_2224 | YdiF (EC 2.8.3.8) | diazotrophicus |
| | | | | (strain ATCC |
| | | | | 49037/DSM |
| | | | | 5601/PAI5) |
| M7PTY6 | | G000_09783 | Acetate CoA-transferase | Klebsiella |
| | | Kpn2146_2726 | YdiF (EC 2.8.3.8) | pneumoniae |
| | | | | ATCC BAA- |
| | | | | 2146 |
| Q2RJ16 | 3833054 | Moth_1259 | Acetate CoA-transferase | Moorella |
| | | | YdiF (EC 2.8.3.8) | thermoacetica |
| | | | | (strain ATCC |
| | | | | 39073) |
| A0QZW0 | 4534496 | MSMEG_4168 | Acetate CoA-transferase | Mycobacterium |
| | | MSMEI_4070 | YdiF (EC 2.8.3.8) | smegmatis |
| | | | | (strain ATCC |
| | | | | 700084/ |
| | | | | mc(2)155) |
| B6VK66 | 13861069 | atoD | Acetate CoA-transferase | Photorhabdus |
| | | PAU_01020 | subunit alpha (EC 2.8.3.8) | asymbiotica |
| | | PA- | (Acetate coa-transferase | subsp. |
| | | RVA1- | subunit alpha (Ec 2.8.3.8) | asymbiotica |
| | | 4466 | (Acetyl coa:acetoacetate | (strain ATCC |
| | | | coa transferase subunit | 43949/3105- |
| | | | alpha)) (EC 2.8.3.8) | 77) |
| | | | | (Xenorhabdus |
| | | | | luminescens |
| | | | | (strain 2)) |
| B6VK67 | 13863234 | atoA | Acetate coa-transferase | Photorhabdus |
| | | PAU_01019 | beta subunit (Acetyl- | asymbiotica |
| | | PA- | coa:acetoacetate co | subsp. |
| | | RVA1- | transferase beta subunit) | asymbiotica |
| | | 4467 | (EC 2.8.3.8) | (strain ATCC |
| | | | | 43949/3105- |
| | | | | 77) |
| | | | | (Xenorhabdus |
| | | | | luminescens |
| | | | | (strain 2)) |
| Q8Y265 | 1219275 | RSc0471 | Acetate CoA-transferase | Ralstonia |
| | | | YdiF (EC 2.8.3.8) | solanacearum |
| | | | | (strain |
| | | | | GMI1000) |
| | | | | (Pseudomonas |
| | | | | solanacearum) |
| F6G5B4 | 12627030 | mdcA | Acetate CoA-transferase | Ralstonia |
| | | RSPO_c02923 | YdiF (EC 2.8.3.8) | solanacearum |
| | | | | (strain Po82) |
| Q92YU3 | 1235806 | SMa1409 | Acetate CoA-transferase | Rhizobium |
| | | | YdiF (EC 2.8.3.8) | meliloti (strain |
| | | | | 1021) (Ensifer |
| | | | | meliloti) |
| | | | | (Sinorhizobium |
| | | | | meliloti) |
| Q92KZ4 | 1234998 | R03304 | Acetate CoA-transferase | Rhizobium |
| | | SMc04399 | YdiF (EC 2.8.3.8) | meliloti (strain |
| | | | | 1021) (Ensifer |
| | | | | meliloti) |
| | | | | (Sinorhizobium |
| | | | | meliloti) |
| Q8ZPR5 | | ydiF | Acetate CoA-transferase | Salmonella |
| | | STM1357 | YdiF (EC 2.8.3.8) | typhimurium |
| | | | | (strain LT2/ |
| | | | | SGSC1412/ |
| | | | | ATCC 700720) |
| E1WFM4 | | ydiF | Acetate CoA-transferase | Salmonella |
| | | SL1344_1291 | YdiF (EC 2.8.3.8) | typhimurium |
| | | | | (strain |
| | | | | SL1344) |
| U2NIA4 | | L581_2735 | Acetate CoA-transferase | Serratia |
| | | | YdiF (EC 2.8.3.8) | fonticola AU- |
| | | | | AP2C |
| U2NF66 | | L581_4507 | Acetate CoA-transferase | Serratia |
| | | | YdiF (EC 2.8.3.8) | fonticola AU- |
| | | | | AP2C |
| U2NBM1 | | L580_1393 | Acetate CoA-transferase | Serratia |
| | | | YdiF (EC 2.8.3.8) | fonticola AU- |
| | | | | P3(3) |
| W8UTY0 | | CH52_04550 | Acetate CoA-transferase | Staphylococcus |
| | | DA92_00435 | YdiF (EC 2.8.3.8) | aureus |
| D3ES62 | 12863675 | SA2981_0235 | Acetate CoA-transferase | Staphylococcus |
| | | | YdiF (EC 2.8.3.8) | aureus |
| | | | | (strain 04- |
| | | | | 02981) |

Additional genes coding for enzymes for application to the synthesis of acetyl CoA from acetate (Activity F) can be identified based on sequence homology to those examples in Table 5.

### Example 2—Conversion of Acetaldehyde to 3-Hydroxybutanal

The syntheses described herein via the intermediate compound 3-hydroxybutanal. Synthesis of 3-hydroxybutanal is achieved using an enzyme capable of the aldol condensation of two molecules of acetaldehyde. This reaction is catalysed by deoxyribose phosphate aldolase (DERA, EC 4.1.2.4). This aldolase can be sourced from a wide range of microorganisms with the example from E. coli having been studied in detail for the synthesis of statin intermediates.

The NIH Genbank® database of publicly available nucleotide sequences (http://www.ncbi.nlm.nih.gov/gene) may be used to identify genes encoding proteins classified as EC 4.1.2.4. Bacterial genes annotated with EC 4.1.2.4 number 1137 as of 6 Jul. 2014; by phylum, there are 394 examples in the firmicutes, 387 in proteobacteria, 153 in actinobacteria, 50 in cyanobacteria and 153 in others. There are also 52 archaeal genes annotated with EC 4.1.2.4. These data are summarised in Table 6.

TABLE 6
Distribution of genes annotated with EC 4.1.2.4 in the Bacteria and Archaea
identified using the NIH Genbank® database of publicly available nucleotide sequences;
accessed 6th Jul. 2014. Example sequences for conversion of acetaldehyde to 3-
hydroxybutanal (Activity G).

| Domain | Phylum | Order | Number of genes |
|---|---|---|---|
| Bacteria | | | 1137 |
| | Firmicutes | | 394 |
| | | Bacillales | 168 |
| | | Lactobacillales | 110 |
| | | Clostridiales | 78 |
| | | Thermoanaerobacterales | 26 |
| | | Halanaerobiales | 6 |
| | | Selenomonadales | 4 |
| | | Natranaerobiales | 1 |
| | | Erysipelotrichales | 1 |
| | Proteobacteria | | 387 |
| | | g-proteobacteria | 276 |
| | | a-proteobacteria | 61 |
| | | b-proteobacteria | 36 |
| | | d-proteobacteria | 14 |
| | Actinobacteria | | 153 |
| | | Actinomycetales | 146 |
| | | high GC Gram+ | 7 |
| | Cyanobacteria | | 50 |
| Archaea | | | 52 |

| UniProt | NCBI Gene | | | |
|---|---|---|---|---|
| Entry | ID | Gene names | Protein names | Organism |
| H6LE06 | 11871631; | deoC4 | Deoxyribose- | Acetobacterium |
| | | Awo_c12650 | phosphate aldolase | woodii (strain ATCC |
| | | | DeoC4 (EC 4.1.2.4) | 29683/DSM 1030/ |
| | | | | JCM 2381/KCTC |
| | | | | 1655) |
| H6LE04 | 11871629; | deoC2 | Deoxyribose- | Acetobacterium |
| | | Awo_c12630 | phosphate aldolase | woodii (strain ATCC |
| | | | DeoC2 (EC 4.1.2.4) | 29683/DSM 1030/ |
| | | | | JCM 2381/KCTC |
| | | | | 1655) |
| H6LF13 | 11870761; | deoC1 deoC | Deoxyribose- | Acetobacterium |
| | | Awo_c01090 | phosphate aldolase | woodii (strain ATCC |
| | | | (DERA) (EC 4.1.2.4) | 29683/DSM 1030/ |
| | | | (2-deoxy-D-ribose 5- | JCM 2381/KCTC |
| | | | phosphate aldolase) | 1655) |
| | | | (Phosphodeoxyriboaldolase) | |
| H6LFY1 | 11871799; | deoC5 | Deoxyribose- | Acetobacterium |
| | | Awo_c14870 | phosphate aldolase | woodii (strain ATCC |
| | | | DeoC5 (EC 4.1.2.4) | 29683/DSM 1030/ |
| | | | | JCM 2381/KCTC |
| | | | | 1655) |
| H6LE05 | 11871630; | deoC3 | Deoxyribose- | Acetobacterium |
| | | Awo_c12640 | phosphate aldolase | woodii (strain ATCC |
| | | | DeoC3 (EC 4.1.2.4) | 29683/DSM 1030/ |
| | | | | JCM 2381/KCTC |
| | | | | 1655) |
| C9RDA8 | 8491097 | deoC | Deoxyribose- | Ammonifex degensii |
| | | Adeg_1109 | phosphate aldolase | (strain DSM 10501/ |
| | | | (DERA) (EC 4.1.2.4) | KC4) |
| | | | (2-deoxy-D-ribose 5- | |
| | | | phosphate aldolase) | |
| | | | (Phosphodeoxyriboaldolase) | |
| P39121 | 938608 | deoC dra | Deoxyribose- | Bacillus subtilis (strain |
| | | BSU39420 | phosphate aldolase | 168) |
| | | | (DERA) (EC 4.1.2.4) | |
| | | | (2-deoxy-D-ribose 5- | |
| | | | phosphate aldolase) | |
| | | | (Phosphodeoxyriboaldolase) | |
| | | | (Deoxyriboaldolase) | |
| Q97IU5 | 1117728 | deoC | Deoxyribose- | Clostridium |
| | | CA_C1545 | phosphate aldolase | acetobutylicum (strain |
| | | | (DERA) (EC 4.1.2.4) | ATCC 824/DSM 792/ |
| | | | (2-deoxy-D-ribose 5- | JCM 1419/LMG |
| | | | phosphate aldolase) | 5710/VKM B-1787) |
| | | | (Phosphodeoxyriboaldolase) | |
| | | | (Deoxyriboaldolase) | |
| Q8NTC4 | 1021418 | deoC Cgl0383 | Deoxyribose- | Corynebacterium |
| | | cg0458 | phosphate aldolase | glutamicum (strain |
| | | | (DERA) (EC 4.1.2.4) | ATCC 13032/DSM |
| | | | (2-deoxy-D-ribose 5- | 20300/JCM 1318/ |
| | | | phosphate aldolase) | LMG 3730/NCIMB |
| | | | (Phosphodeoxyriboaldolase) | 10025) |
| | | | (Deoxyriboaldolase) | |
| P0A6L0 | 12934356; | deoC dra thyR | Deoxyribose- | Escherichia coli |
| | 948902 | b4381 | phosphate aldolase | (strain K12) |
| | | JW4344 | (DERA) (EC 4.1.2.4) | |
| | | | (2-deoxy-D-ribose 5- | |
| | | | phosphate aldolase) | |
| | | | (Phosphodeoxyriboaldolase) | |
| | | | (Deoxyriboaldolase) | |
| A4IR26 | 4967361 | deoC | Deoxyribose- | Geobacillus |
| | | GTNG_2435 | phosphate aldolase | thermodenitrificans |
| | | | (DERA) (EC 4.1.2.4) | (strain NG80-2) |
| | | | (2-deoxy-D-ribose 5- | |
| | | | phosphate aldolase) | |
| | | | (Phosphodeoxyriboaldolase) | |
| | | | (Deoxyriboaldolase) | |
| Q8ZXK7 | 1465578 | deoC | Deoxyribose- | Pyrobaculum |
| | | PAE1231 | phosphate aldolase | aerophilum (strain |
| | | | (DERA) (EC 4.1.2.4) | ATCC 51768/IM2/ |
| | | | (2-deoxy-D-ribose 5- | DSM 7523/JCM |
| | | | phosphate aldolase) | 9630/NBRC 100827) |
| | | | (Phosphodeoxyriboaldolase) | |
| | | | (Deoxyriboaldolase) | |
| Q8ZJV8 | 1256093 | deoC | Deoxyribose- | Salmonella |
| | | STM4567 | phosphate aldolase | typhimurium (strain |
| | | | (DERA) (EC 4.1.2.4) | LT2/SGSC1412/ |
| | | | (2-deoxy-D-ribose 5- | ATCC 700720) |
| | | | phosphate aldolase) | |
| | | | (Phosphodeoxyriboaldolase) | |
| | | | (Deoxyriboaldolase) | |
| P99174 | 1124840 | deoC2 | Deoxyribose- | Staphylococcus |
| | | SA1939 | phosphate aldolase 2 | aureus (strain N315) |
| | | | (DERA 2) (EC | |
| | | | 4.1.2.4) (2-deoxy-D- | |
| | | | ribose 5-phosphate | |
| | | | aldolase 2) | |
| | | | (Phosphodeoxyriboaldolase | |
| | | | 2) | |
| | | | (Deoxyriboaldolase 2) | |
| Q99Y51 | 3571313; | deoC | Deoxyribose- | Streptococcus |
| | 902077 | SPy_1867 | phosphate aldolase | pyogenes serotype |
| | | M5005_Spy1585 | (DERA) (EC 4.1.2.4) | M1 |
| | | | (2-deoxy-D-ribose 5- | |
| | | | phosphate aldolase) | |
| | | | (Phosphodeoxyriboaldolase) | |
| | | | (Deoxyriboaldolase) | |
| Q9X1P5 | 897566 | deoC | Deoxyribose- | Thermotoga maritima |
| | | TM_1559 | phosphate aldolase | (strain ATCC 43589/ |
| | | | (DERA) (EC 4.1.2.4) | MSB8/DSM 3109/ |
| | | | (2-deoxy-D-ribose 5- | JCM 10099) |
| | | | phosphate aldolase) | |
| | | | (Phosphodeoxyriboaldolase) | |
| | | | (Deoxyriboaldolase) | |
| Q72JE9 | 2775585 | deoC | Deoxyribose- | Thermus |
| | | TT_C0823 | phosphate aldolase | thermophilus (strain |
| | | | (DERA) (EC 4.1.2.4) | HB27/ATCC BAA- |
| | | | (2-deoxy-D-ribose 5- | 163/DSM 7039) |
| | | | phosphate aldolase) | |
| | | | (Phosphodeoxyriboaldolase) | |
| | | | (Deoxyriboaldolase) | |
| Q8ZGH4 | 1147807; | deoC1 dra | Deoxyribose- | Yersinia pestis |
| | 1174165; | YPO1323 | phosphate aldolase 1 | |
| | 2764428 | y2860 | (DERA 1) (EC | |
| | | YP_1269 | 4.1.2.4) (2-deoxy-D- | |
| | | | ribose 5-phosphate | |
| | | | aldolase 1) | |
| | | | (Phosphodeoxyriboaldolase | |
| | | | 1) | |
| | | | (Deoxyriboaldolase 1) | |
| A2BLE9 | 4781378 | Hbut_0962 | Deoxyribose- | Hyperthermus |
| | | | phosphate aldolase | butylicus (strain DSM |
| | | | (DERA) (EC 4.1.2.4) | 5456/JCM 9403) |
| C0ZUQ6 | 7712431 | deoC, | Deoxyribose- | Rhodococcus |
| | | RER_15930 | phosphate aldolase | erythropolis (strain |
| | | | (DERA) (EC 4.1.2.4) | PR4/NBRC 100887) |
| B4A422 | | deoC | Deoxyribose- | Salmonella enterica |
| | | SNSL317_A2005 | phosphate aldolase | subsp. enterica |
| | | | (DERA) (EC 4.1.2.4) | serovar Newport str. |
| | | | | SL317 |
| Q88264 | 1061480 | deoC lp_0497 | Deoxyribose- | Lactobacillus |
| | | | phosphate aldolase | plantarum (strain |
| | | | (DERA) (EC 4.1.2.4) | ATCC BAA-793/ |
| | | | | NCIMB 8826/ |
| | | | | WCFS1) |
| S8F6M2 | 7901233 | TGME49_318750 | Deoxyribose- | Toxoplasma gondii |
| | | | phosphate aldolase | ME49 |
| | | | (DERA) (EC 4.1.2.4) | |
| Q87710 | 3233914 | deoC TK2104 | Deoxyribose- | Thermococcus |
| | | | phosphate aldolase | kodakaraensis (strain |
| | | | (DERA) (EC 4.1.2.4) | ATCC BAA-918/JCM |
| | | | | 12380/KOD1) |
| | | | | (Pyrococcus |
| | | | | kodakaraensis (strain |
| | | | | KOD1)) |
| B9DS93 | 7393055 | deoC SUB0952 | Deoxyribose- | Streptococcus uberis |
| | | | phosphate aldolase | (strain ATCC BAA- |
| | | | (DERA) (EC 4.1.2.4) | 854/0140J) |
| A4WHP | 5054261 | deoC | Deoxyribose- | Pyrobaculum |
| | | Pars_0301 | phosphate aldolase | arsenaticum (strain |
| | | | (DERA) (EC 4.1.2.4) | DSM 13514/JCM |
| | | | | 11321) |
| A1RU26 | 4617152 | deoC | Deoxyribose- | Pyrobaculum |
| | | Pisl_1295 | phosphate aldolase | islandicum (strain |
| | | | (DERA) (EC 4.1.2.4) | DSM 4184/JCM |
| | | | | 9189) |
| C4M5C6 | 3406093 | EHI_121800 | Deoxyribose- | Entamoeba histolytica |
| | | | phosphate aldolase | |
| | | | (DERA) putative | |
| A8A8B0 | 5593924 | deoC | Deoxyribose- | Escherichia coli |
| | | EcHS_A4616 | phosphate aldolase | O9:H4 (strain HS) |
| | | | (DERA) (EC 4.1.2.4) | |
| Q0SEY5 | 4218140 | deoC | Deoxyribose- | Rhodococcus sp. |
| | | RHA1_ro02094 | phosphate aldolase | (strain RHA1) |
| | | | (DERA) (EC 4.1.2.4) | |
| F8K193 | 11354892 | Dera, | Deoxyribose- | Streptomyces cattleya |
| | 12650565 | SCAT_3805, | phosphate aldolase | (strain ATCC 35852/ |
| | | SCATT_37940 | | DSM 46488/JCM |
| | | | | 4925/NBRC 14057/ |
| | | | | NRRL 8057) |
| A7FU73 | 5395000 | deoC, | Deoxyribose- | Clostridium botulinum |
| | | CLB_1583 | phosphate aldolase | (strain ATCC 19397/ |
| | | | | Type A) |
| B9E4U5 | 7273626 | deoC, | Deoxyribose- | Clostridium kluyveri |
| | | CKR_2469 | phosphate aldolase | (strain NBRC 12016) |
| D8GI14 | 9445430 | deoC | Deoxyribose- | Clostridium ljungdahlii |
| | | CLJU_c18130 | phosphate aldolase | (strain ATCC 55383/ |
| | | | | DSM 13528/PETC) |
| E3GHB9 | 9881953 | deoC | Deoxyribose- | Eubacterium limosum |
| | | ELI_0052 | phosphate aldolase | (strain KIST612) |
| B5Y277 | 6936643 | deoC | Deoxyribose- | Klebsiella |
| | | KPK_4777 | phosphate aldolase | pneumoniae (strain |
| | | | | 342) |
| Q5KY02 | 3184692 | deoC | Deoxyribose- | Geobacillus |
| | | GK_2499 | phosphate aldolase | kaustophilus (strain |
| | | | | HTA426) |
| Q6HK62 | 2856540 | deoC | Deoxyribose- | Bacillus thuringiensis |
| | | BT9727_1732 | phosphate aldolase | subsp. konkukian |
| | | | | (strain 97-27) |
| I3DVS3 | | deoC | Deoxyribose- | Bacillus methanolicus |
| | | PB1_12319 | phosphate aldolase | PB1 |

Additional genes coding for enzymes capable of the condensation of two molecules of acetaldehyde (Activity G) can be identified based on sequence homology to those examples in Table 6.

### Example 3—Reduction of 3-Hydroxybutanal to 1,3-Butanediol

Genes coding for enzymes capable of the reduction of an aldehyde to the corresponding alcohol (EC 1.1.1.-) are widespread in nature and with respect to this application are generally classified in EC 1.1.1.78; 1.1.1.265; 1.1.1.373; 1.1.1.1; 1.1.1.2; 1.1.1.21; 1.1.1.26; 1.1.1.31; 1.1.1.71; 1.1.1.72; 1.1.1.77 and 1.1.1.283 For this application it is desirable that aldehyde reductase or alcohol dehydrogenases enzymes (both terms refer to enzymes capable of aldehyde reduction) show preference towards a C4 aldehyde relative to a C2 aldehyde such as acetaldehyde. Alcohol dehydrogenases involved in ethanol synthesis for example, preferring acetaldehyde as a substrate would not be preferred for this application, but evolution of these well described short chain dehydrogenase or reductases using techniques well known in the art, could be used to alter the substrate preference towards longer chain aldehydes.

This reaction can be catalysed by a medium chain alcohol dehydrogenase which showed preference for alcohols of C4 or greater, for example (gene alrA) see Appl. Environ. Microbiol, 2000, 66, 5231. Further, alcohol dehydrogenases showing preference for longer chain alcohols from Acinebacter calcoaceticus NCIB 8250 and from Saccharomyces cerevisiae D273-10B are described by Wales, M and Fewson, C. Microbiol 1994, 140, 173. Although measured in the oxidative direction, the dehydrogenase also accepts 1,4-butanediol as a substrate. 2,3-butanediol is not a substrate, clearly demonstrating the desired primary alcohol as opposed to secondary alcohol specificity for application to 3-hydroxybutanal reduction.

A further excellent candidate enzyme is bcALD, GRE_2 (EC 1.1.1.265 also classified in EC 1.1.1.283) from S. cerevisiae var. uvarum W34 described by van Iersel, M. F. M et al. Appl. Environ. Microbiol. 1997. 63, 4079. This enzyme shows strong preference for butanal and derivatives with a poor preference for acetaldehyde. Kms are: acetaldehyde 158 mM; butanal 2.76 mM; 2-methylbutanal 1.85 mM; 3-methylbutanal 0.21 mM. The preference of GRE2 derived dehydrogenase for a C4 aldehyde (butanal) relative to acetaldehyde is shown in Example 9. From these data this dehydrogenase would also be expected to show selective preference for 3-hydroxybutanal relative to acetaldehyde.

Another excellent example is the GOX1615 gene from Gluconobacter oxydans (Richter, N. et al. Chembiochem. 2009, 10, 1888.). This enzyme has been characterised and shown to have very poor preference for acetaldehyde reduction compared to longer chain and hydroxysubstituted substrates. 3-Hydroxybutanal was not specifically tested in the reductive direction. However, 1,3-butanediol was tested in the undesired oxidative direction and poor activity was reported. Hence, based on the data presented it is expected that this enzyme would show the desired preference for 3-hydroxybutanal reduction compared to undesired acetaldehyde reduction also with a poor oxidative activity towards the product 1,3-butanediol. The use of GOX1615 for selective reduction of 3-hydroxybutanal to 1,3-butanediol within a novel, unnatural 1,3-BDO pathway, is shown in Example 12,13 and 14. Example 9 also confirms its predicted preference for the DERA product 3-hydroxybutanal relative to acetaldehyde.

Further examples include yqhD from E. coli which is reported as having a preference for alcohols of C3 or greater, Sulzenbacher et al., 2004. J. Mol. Biol. 342:489-502. Alcohol dehydrogenases are understood to be reversible enzymes capable of operating in a reductive or oxidative direction. Genes bdh A and bdh B (proteins bdh I and bdh II) from C. acetobutylicum code for enzymes which convert butanal into butanol (Walter et al. 1992. J. Bacteriol. 174:7149-7158. The use of bdhII (gene bdhB) butanol dehydrogenase for reduction of 3-hydroxybutanal to 1,3-butanediol within a novel, unnatural 1,3-BDO pathway, is shown in Example 12. Further, butanol dehydrogenase examples include bdh from C. saccharoperbutylacetonicum and Cbei_1722, Cbei_2181 and Cbei_2421 in C. Beijerincki (Gene announce. 2012, 194, (19) 5470.). Other gene products classified as methylglyoxal reductases (EC 1.1.1.283) in addition to GRE_2 described above, may also be candidates (Eur. J. Biochem, 1988, 171,213). Additional aldehyde reductase gene candidates in Saccharomyces cerevisiae include the aldehyde reductases GRE3, ALD2-6 and HFD1, glyoxylate reductases GOR1 and YPL113C and glycerol dehydrogenase GCY1 (Atsumi et al., Nature 451:86-89 2008).

TABLE 7
Examples of genes expressing enzymes for the conversion of 3-hydroxybutanal
to 1,3-butanediol (Activity H).

| UniProt | NCBI Gene | | | |
|---|---|---|---|---|
| Entry | ID | Gene names | Protein names | Organism |
| Q9F1R1 | | alrA | NADPH-dependent | Acinetobacter |
| | | | alcohol | sp. M-1 |
| | | | dehydrogenase (EC | |
| | | | 1.1.1.2) | |
| Q04944 | 1119481 | bdhA | NADH-dependent | Clostridium |
| | | CA_C3299 | butanol | acetobutylicum |
| | | | dehydrogenase A | (strain ATCC |
| | | | (EC 1.1.1.—) (BDH I) | 824/DSM 792/ |
| | | | | JCM 1419/LMG |
| | | | | 5710/VKM B- |
| | | | | 1787) |
| Q04945 | 1119480 | bdhB | NADH-dependent | Clostridium |
| | | CA_C3298 | butanol | acetobutylicum |
| | | | dehydrogenase B | (strain ATCC |
| | | | (EC 1.1.1.—) (BDH II) | 824/DSM 792/ |
| | | | | JCM 1419/LMG |
| | | | | 5710/VKM B- |
| | | | | 1787) |
| Q46856 | 12933386; | yqhD b3011 | Alcohol | Escherichia coli |
| | 947493 | JW2978 | dehydrogenase YqhD | (strain K12) |
| | | | (EC 1.1.1.—) | |
| Q12068 | 854014; | GRE2 | NADPH-dependent | Saccharomyces |
| | | YOL151W | methylglyoxal | cerevisiae (strain |
| | | | reductase GRE2 (EC | ATCC 204508/ |
| | | | 1.1.1.283) (3- | S288c) (Baker's |
| | | | methylbutanal | yeast) |
| | | | reductase) (EC | |
| | | | 1.1.1.265) (Genes de | |
| | | | respuesta a estres | |
| | | | protein 2) | |
| | | | (Isovaleraldehyde | |
| | | | reductase) | |
| P00331 | 855349 | ADH2 ADR2 | Alcohol | Saccharomyces |
| | | YMR303C | dehydrogenase 2 (EC | cerevisiae (strain |
| | | YM9952.05C | 1.1.1.1) (Alcohol | ATCC 204508/ |
| | | | dehydrogenase II) | S288c) (Baker's |
| | | | (YADH-2) | yeast) |
| P0A9S1 | 12930229; | fucO b2799 | Lactaldehyde | Escherichia coli |
| | 947273 | JW2770 | reductase (EC | (strain K12) |
| | | | 1.1.1.77) | |
| | | | (Propanediol | |
| | | | oxidoreductase) | |
| P20368 | 3188393 | adhA | Alcohol | Zymomonas |
| | | ZMO1236 | dehydrogenase 1 (EC | mobilis subsp. |
| | | | 1.1.1.1) (Alcohol | mobilis (strain |
| | | | dehydrogenase I) | ATCC 31821/ |
| | | | (ADH I) | ZM4/CP4) |
| A2PYM4 | | bdh | Butanol | Clostridium |
| | | | dehydrogenase | saccharoperbutyl |
| | | | | acetonicum |
| A6LU64 | 5292938 | Cbei_1722 | Iron-containing | Clostridium |
| | | | alcohol | beijerinckii |
| | | | dehydrogenase | (strain ATCC |
| | | | | 51743/NCIMB |
| | | | | 8052) |
| | | | | (Clostridium |
| | | | | acetobutylicum) |
| A6LVG8 | 5293392 | Cbei_2181 | Iron-containing | Clostridium |
| | | | alcohol | beijerinckii |
| | | | dehydrogenase | (strain ATCC |
| | | | | 51743/NCIMB |
| | | | | 8052) |
| | | | | (Clostridium |
| | | | | acetobutylicum) |
| A6LW49 | 5293624 | Cbei_2421 | Iron-containing | Clostridium |
| | | | alcohol | beijerinckii |
| | | | dehydrogenase | (strain ATCC |
| | | | | 51743/NCIMB |
| | | | | 8052) |
| | | | | (Clostridium |
| | | | | acetobutylicum) |
| P38715 | 856504 | GRE3 | NADPH-dependent | Saccharomyces |
| | | YHR104W | aldose reductase | cerevisiae (strain |
| | | | GRE3 (EC 1.1.1.21) | ATCC 204508/ |
| | | | (NADPH-dependent | S288c) (Baker's |
| | | | methylglyoxal | yeast) |
| | | | reductase GRE3) | |
| | | | (Xylose reductase) | |
| | | | (EC 1.1.1.—) | |
| Q5FQJ0 | 3248904 | GOX1615 | Putative | Gluconobacter |
| | | | oxidoreductase (EC | oxydans (strain |
| | | | 1.1.1.—) | 621H) |
| | | | | (Gluconobacter |
| | | | | suboxydans) |
| P28811 | 879097 | mmsB, | EC 1.1.1.31 | Pseudomonas |
| | | PA3569 | 3-hydroxyisobutyrate | aeruginosa |
| | | | dehydrogenase | (strain ATCC |
| | | | | 15692/PAO1/ |
| | | | | 1C/PRS 101/ |
| | | | | LMG 12228) |
| Q5SLQ6 | 3168163 | TTHA0237 | EC 1.1.1.31 | Thermus |
| | | | 3-hydroxyisobutyrate | thermophilus |
| | | | dehydrogenase | (strain HB8/ |
| | | | | ATCC 27634/ |
| | | | | DSM 579) |
| A6LU64 | 5292938 | Cbei_1722 | Iron-containing | Clostridium |
| | | | alcohol | beijerinckii |
| | | | dehydrogenase. | (strain ATCC |
| | | | | 51743/NCIMB |
| | | | | 8052) |
| | | | | (Clostridium |
| | | | | acetobutylicum) |
| B3LMK7 | | SCRG_02216 | Medium chain alcohol | Saccharomyces |
| | | | dehydrogenase | cerevisiae (strain |
| | | | | RM11-1a) |
| | | | | (Baker's yeast) |
| C4QWW0 | 8196620 | PAS_chr1- | Medium chain alcohol | Komagataella |
| | | 1_0357 | dehydrogenase | pastoris (strain |
| | | | (NADPH) | GS115/ATCC |
| | | | | 20864) (Yeast) |
| | | | | (Pichia pastoris) |
| A6ZTC9 | | ADH7, | Medium chain alcohol | Saccharomyces |
| | | SCY_0522 | dehydrogenase | cerevisiae (strain |
| | | | | YJM789) |
| | | | | (Baker's yeast) |
| P27250 | 12934055 | Ahr yjgB | Aldehyde reductase | Escherichia coli |
| | 948802 | b4269, | | (strain K12) |
| | | JW5761 | | |

Additional genes coding for enzymes capable of the conversion of 3-hydroxybutanal to 1,3-butanediol (Activity H) can be identified based on sequence homology to those examples in Table 7.

### Example 4—Culture of Acetogen Strains for Production of 1,3-Butanediol or Other Downstream Product

For the production of downstream products of 3-hydroxybutanal such as 1,3-butanediol the recombinant acetogen strain may be cultured in a defined, semi-defined or undefined medium supplemented with syngas as the only or principle carbon and energy source is well known in the art. Examples of additional sources of energy or carbon may be nitrate, methanol or sugar. It is highly desirable to maintain anaerobic conditions as the acetogen strains of the present Example are strict anaerobes. Initial tests with the wild type organism and with genetically modified organisms before moving to a fermenter can be done in small bottles that are fitted with thick rubber stoppers and aluminium crimps employed to seal the bottles and as those skilled in the art will understand.

Suitable replicates such as triplicate cultures can be grown for each engineered strain and culture supernatants can be tested for products formed. For example, syngas composition in the media, metabolic intermediates, 1,3-butanediol and by-product(s) formed in the engineered production host can be measured as a function of time and can be analysed by methods such as High Performance Liquid Chromatography (HPLC), GC (Gas Chromatography), GC-MS (Gas Chromatography-Mass Spectroscopy) and LC-MS (Liquid Chromatography-Mass Spectroscopy) or other suitable analytical methods using routine procedures well known in the art.

Acetate, pyruvate, acetyl-Co A, 3-hydroxybutanal, 1,3-butanediol and intermediates or other desired products can be quantified by HPLC using as appropriate, a refractive index detector or UV detector or other suitable assay and detection methods well known in the art. The individual enzyme or protein activities expressed from the heterologous DNA sequences or overexpressed endogenous DNA sequences, can also be assayed using methods well known in the art.

Fermentations can be performed in continuous cultures, batch or fed-batch. All of these processes are well known in the art. Important process considerations for syngas fermentation are high biomass concentration and good gas-liquid mass transfer Bredwell et al, (1999), Biotechnol. Prog. 15:834-844. As carbon monoxide has a lower solubility in water compared to oxygen, continuously gas-sparged fermentations are recommended and can be performed in controlled fermentors with constant off-gas analysis by mass spectrometry and periodic liquid sampling and analysis discussed above. Other feedstocks such as methanol or sugar can be fed to the fermentor using traditional approaches.

### Example 5. Generation of a Lactate Dehydrogenase Gene Knockout in Acetobacterium woodii

### Plasmids and Primers

| | | Restriction |
|---|---|---|
| Primer | Sequence | site |
| EryFor | ggGGATCCAATG | BamHI |
| | ATACACCAATCA | |
| | GTGC | |
| EryXbaRev | ggTCTAGATTGA | XbaI |
| | ACCCGTCTCCTT | |
| | ACG | |
| 01For | ggGAATTCatgt | EcoRI |
| (uplacdehyEco) | cgatcatattga | |
| | agg | |
| 01Rev | aaGGATCCctta | BamH1 |
| (uplacdehyBam) | aacgaccatcc | |
| 02For | cctctagagtga | XbaI |
| (dwnlacdehyXba) | gatgatggataa | |
| | c | |
| 02Rev | gcAAGCTTCTGC | PstI, |
| (dwnlacdehyPstHind) | AGtcatccaaat | HindIII |
| | gtgcttaacaac | |
| | c | |
| 16For | aaTCTAGAttag | XbaI |
| | tcggtgcctgtg | |
| 16Rev | ggAAGCTTtctt | HindIII |
| | tacgttctacg | |
| 13b | aaACTAGTggcg | SpeI |
| (LDHantiFor50) | attccaacggcg | |
| | at | |
| 13 | aaCTCGAGctac | XhoI |
| (LDHantiRev) | atctgacagact | |
| | tttttcgg | |
| pAMβ1For | CGATTTCCGATT | — |
| | GATTGCTT | |
| pAMβ1Rev | AAT CCC AAA | — |
| | TGA GCC AACAG | |

| Plasmid | Description |
|---|---|
| pUC19 | ampicillin resistance, suitable for blue white |
| | cloning |
| pTRKH2 | Erythromycin resistance, replicon for gram+ and |
| | E. coli |
| pUC19-Ery(XbaI) | pUC19 vector containing the Erythromycin |
| (pE) | resistance gene in the BamHI and XbaI site |
| pUC19-Ery-LDHup- | pUC19 vector containing the Erythromycin |
| LDHdown | resistance gene in the BamHI and XbaI site and |
| (pE01-02) | the LDH upstream region cloned in EcoRI-BamHI |
| | and LDH downstream region of cloned into |
| | XbaI-HindIII |
| pUC19-Ery-LDHk/o | pUC19 vector containing the Erythromycin |
| (pE16) | resistance gene in the BamHI and XbaI site and |
| | a 728 bp fragment of the LDH gene |
| pUC19-pAMβ1-Ery | pUC19 vector containing the gram+ replicon |
| (pEP) | pAMβ1 in the EcoRI and KpnI site, Erythromycin |
| | resistance gene in the BamHI and XbaI |

Media for Acetobacterium woodii

The medium was prepared using anaerobic techniques and contained under an N₂—CO₂ atmosphere (80:20) the following per 1000 ml:

| | | |
|---|---|---|
| KH₂PO₄ | 1.76 | g |
| K₂HPO₄ | 8.44 | g |
| NH₄Cl | 1.0 | g |
| cysteine hydrochloride | 0.5 | g |
| MgSO₄ × 6H₂O | 0.33 | g |
| NaCl | 2.9 | g |
| yeast extract | 2.0 | g |
| KHCO₃ | 6.0 | g |
| Resazurin | 0.001 | g |
| Trace element solution SL 9 | 1.0 | ml |
| selenite-tungstate solution | 1.0 | ml |
| vitamin solution DSMZ 141 | 2.0 | ml |
| Carbon source | 20-40 | mM |

Carbon source and magnesium were added after autoclaving from an anaerobe, sterile stock solution (2 M Fructose or Lactate and 0.75 M respectively).

For solid media 15 g/L agar was added and the media boiled to remove dissolved oxygen and then cooled down under a stream of gas, before autoclaving.

### Vitamin Solution

| | |
|---|---|
| Biotin | 2.00 mg |
| Folic acid | 2.00 mg |
| Pyridoxine-HCl | 10.00 mg |
| Thiamine-HCl × 2H₂O | 5.00 mg |
| Riboflavin | 5.00 mg |
| Nicotinic acid | 5.00 mg |
| D-Ca-pantothenate | 5.00 mg |
| Vitamin B12 | 0.10 mg |
| p-Aminobenzoic acid | 5.00 mg |
| Lipoic acid | 5.00 mg |
| 1000 ml dH₂O | |

### Selenite-Tungstate Solution

| | |
|---|---|
| NaOH | 0.5 g |
| Na₂SeO₃ × 5H₂O | 3 mg |
| Na₂WO₄ × 2H₂O | 4 mg |
| 1000 ml dH2O | |

### Trace Element Solution SL 9

| | |
|---|---|
| Nitrilotriacetic acid | 12.8 g |
| FeCl₂ × 4H₂O | 2.0 g |
| ZnCl₂ | 0.070 g |
| MnCl₂ × 4H₂O | 0.1 g |
| H₃BO₃ | 0.006 g |
| CoCl₂ × 6H₂O | 0.19 g |
| CuCl₂ × 2H₂O | 0.002 g |
| NiCl₂ × 6H₂O | 0.024 g |
| Na₂MoO4 × 2H₂O | 0.036 g |

1000 ml dH₂O, Nitrilotriacetic acid was dissolved first and the pH adjusted to 6.0 with NaOH before all other Trace elements were added.

### Construction of the LDH Knockout Mutant

To construct a Lactate-dehydrogenase knockout mutant two strategies were followed.

(1) Involving two cross-over recombination processes where the full length LDH gene is replaced with the Erythromycin cassette (FIG. 11)
(2) A single recombination event leading to integration of the complete cloning plasmid and therefore interrupting the LDH gene. (FIG. 12)

To construct the plasmids for the LDH knockout mutants of A. woodii pUC19 was used as backbone plasmid. First a erythromycin antibiotic cassette was cloned into the BamHI and XbaI restriction sites yielding the plasmid pUC19-Ery. The Erythromycin resistance cassette was amplified by PCR using gene specific primers (EryFor, EryXbaRev) and plasmid pTRKH₂ as template. Functionality of the antibiotic resistance was confirmed by growth of E. coli DH10B harbouring this plasmid in the presence of 150-300 μg/ml Erythromycin.

To confirm that the plasmid does not integrate into the A. woodii genome randomly an aliquot was used to transform A. woodii. As expected no erythromycin resistance bacteria were obtained after several attempts.

For strategy (1) approximately 1000 bp flanking region upstream and downstream of the LDH gene were amplified using the gene specific primers (01For, 01Rev) for the upstream region and (02For, 02Rev) for the downstream region with genomic DNA as template. The upstream region was cloned into the EcoRI and BamHI region leading to plasmid pUC19-Ery-LDHup which was then used to clone the downstream region into XbaI and HindIII sites yielding plasmid pUC19-Ery-LDHup-LDHdown (Plasmid pE01-02). In strategy (2) a 728 bp fragment of the LDH gene generated by PCR from genomic DNA using primer 16For/16Rev, This PCR products was cloned into pUC19-Ery into XbaI and HindIII site, giving plasmid (pE16).

Plasmid pE01-02 was used to transform A. woodii. All following procedures were carried out under anaerobic conditions. 1 ml of a fresh 5 ml over-night culture was used to inoculate 10 ml media and A. woodii grown to an OD₆₀₀ of approximately 0.5. Cells were spun down in hungate tubes for 10 min at 4000 rpm at 4° C. and washed twice with 10 ml ice-cold anaerobic 270 mM sucrose solution. The pellet was resuspended in 200 μL sucrose and transferred on ice into the anaerobic chamber. 4 μl of each plasmid was added to 40 μl cells, transferred to a 0.2-cm electroporation cuvette and kept on ice for 5 min. For electroporation the following settings were used: electric pulse of 10 kV, electric resistance of 400 Ω and 25 μF. Following electroporation the cells were kept on ice for another 5 min before 960 μl media was added. The transformed cells were incubated o/n at 30° C. and then transferred to 50 ml media containing the required antibiotic (20 μg/ml erythromycin). Single cross-over resistant cells grew within 48 hours. An aliquot of the liquid culture was plated on solid media with the required antibiotic (50 μg/ml erythromycin). Single colonies were obtained after 4-5 days, which were picked and grown up in 5 ml cultures in the presence of erythromycin.

For the double cross-over knockout the plasmid was cut using the restriction enzymes EcoRI and HindIII to obtain an approximately 3837 bp linear fragment containing the erythromycin gene flanked by the up and downstream LDH region. The linear fragment was gel extracted and used to transform A. woodii. The transformation of A. woodii was performed as described above. An erythromycin resistant culture was obtained after a 3-4 days. The culture were plated on solid media and colonies obtained after 5-6 days.

In both cases the colonies were screened for the presence of the erythromycin gene using primers EryFor, EryXbaRev and in the case of the double cross-over for the absence of the LDH gene using primer set 13For/Rev. In the latter case 2 colonies showed the absence of the LDH gene.

Those 2 colonies, as well as 2 colonies for the single cross-over recombination (SR) were picked and analysed for their growth on Fructose and Lactate. Two cultures of A. woodii wild type (Aw1, Aw2) and A. woodii harbouring plasmid pUC19-pAMβ1-Ery (P1, P2) were used as control strains. Plasmid pUC19-pAMβ1-Ery was constructed by cloning the gram positive replicon pAMβ1 from plasmid pTRKH₂ into the EcoRI and KpnI sites of pUC19 and then cloning the Erythromycin cassette into the XbaI-BamHI site. The plasmid was then transformed into A. woodii as described before and resistance growth obtained after 2-3 days. Single colonies were picked and analyzed for the plasmid by PCR using the specific primers for the erythromycin gene (EryFor, EryXbaRev) as well for the pAMβ1 replicon (pAMβ1 For, pAMβ1 Rev).

For the growth curves 500 μl of a fresh over-night culture was used to inoculate a 50 ml anaerobe culture. Either 20 mM Fructose or 40 mM DL-Lactate ((Lactic acid) were used as a substrate. Erythromycin was prepared as a stock concentration of 2 mg/ml in water. One ml samples were taken twice a day over a period of 4 days. Of this 1 ml, 500 μl were used for OD₆₀₀ measurement. The remaining 500 μl were spun down and the supernatant frozen at −20° C. for HPLC analysis.

FIG. 13 shows the obtained growth curve in Fructose media. Here, all mutants grew similar to the control strains (Aw1, Aw2, P1, and P2). In contrast no growth was obtained for the mutants when grown in Lactate, confirming the expected phenotype for the Lactate Dehydrogenase knockout (FIG. 13).

For HPLC analysis 10 μl samples were injected on the HPLC column Rezex ROA Organic Acid H⁺ (300×7.8 mm, Phenomex). The used mobile Phase was 100% 0.01N H₂₅₀₄. Samples were analyzed for 30 minutes with a flow rate of 0.6 ml/min.

The HPLC analysis of those cultures showed that the fructose is consumed and acetate is produced by those mutants at a similar rate and amount (FIG. 14). In contrast, no Lactate is consumed by the mutants, while the control strains utilize it and produce acetate (FIG. 15).

Additionally, the double cross-over knockouts are stable in contrast to the single cross-over mutants (SR). After 5 days a growth on Lactate is observed, probably due to degradation of the Erythromycin which then allows a second recombination step where the cassette is removed from the genome and a functional LDH gene is obtained. HPLC data also confirmed that Lactate is slowly utilized after 5 days by SR1 and SR2 and acetate is produced (data not shown). Those results confirm that the constructed vector can be used to generate stable knockout mutants.

### Example 6. Generation of a Phosphotransacetylase (PTA) Gene Knockout in Acetobacterium woodii

### Primers:

EryFor
ggGGATCCAATGATACACCAATCAGTGC
EryXbaRev
ggTCTAGATTGAACCCGTCTCCTTACG
Pr1_cisFor
ggACTAGTTGTTATTTGGCGATCAGC
Pr4_iorRev
ggCTGCAGCGCACCCATACAAAGC
Pr5_PTAfrag1Rev
AACATCAACATGCGGCCGCACTTACCAAATTATCTGCGTCG
Pr6_PTAfrag3For
AATTTGGTAAGTGCGGCCGCATGTTGATGTTATTCTCATGC

### Plasmids:

pUC19 Ampicillin resistance
pTRKH₂ Erythromycin resistance, replicon for gram+ and E. coli
pUC19-Ery
pUC19-Ery-ΔPTA2

### Strategy

Erythromycin resistance gene was amplified from plasmid pTRKH₂ using primer Ery for and EryXabRev and cloned into pUC19 yielding a non-replicative plasmid, pUC19-Ery. The PTA knock-out cassette was constructed as following: Primers Pr1_cisFor and Pr5_PTAfrag1Rev were used to amplify the upstream region of the PTA gene as well as the N-terminal part of the PTA gene from genomic A. woodii DNA. Pr4_iorRev and Pr6_PTAfrag3For were used to amplify the C-terminal part of the PTA gene as well as the downstream region. This two fragments were cloned together using SOE PCR and primers Pr1_cisFor and Pr4_iorRev. The so constructed knockout cassette harbors a modified PTA gene sequence, consisting of the N and C-terminal part of the PTA gene only. Both parts are separated by a NotI restriction site which was introduced by previous PCR round. The knockout cassette was cloned into XbaI and PstI site of pUC19-Ery, yielding plasmid pUC19-Ery-ΔPTA2

Plasmid pUC19-Ery-ΔPTA2 was used to transform A. woodii. All following procedures were carried out under anaerobic conditions. A 10 ml culture was inoculated with A. woodii and grown to an OD600 of approximately 0.5. Cells were spun down in hungate tubes for 10 min at 4000 rpm at 4° C. and washed twice with 10 ml 10 ice-cold anaerobic 270 mM sucrose solution. The pellet was resuspended in 200 μL sucrose. 4 μl of plasmid was added to 40 μl cells, transferred to a 0.2-cm electroporation cuvette and kept on ice for 5 min. For electroporation the following settings were used: electric pulse of 10 kV, electric resistance of 400 Ω and 25 μF. Following electroporation the cells were kept on ice for another 5 min. The transformed cells were recovered in media, incubated anaerobe for 6 h at 30° C. and then transferred to 50 ml of medium containing the required antibiotic (20 μg/ml erythromycin). The culture was incubated at 30° C. until growth was obtained. An aliquot of the culture was plated on solid medium. Single colonies were obtained after 5-7 days, which were picked and grown up in 10 ml cultures in the presence of erythromycin.

The cultures were genetically analyzed by specific primers to confirm the integration of the plasmid. The culture was passaged into liquid media without erythromycin to allow looping out of the plasmid and generation of a stable PTA knockout via a second recombination event. Passages were plated on solid media until such an event occurred. PTA knockout clones were screened by replica plating in the presence and absence of Erythromycin. Clones not capable of growing in the presence of Erythromycin were picked and analysed for the PTA genotype by PCR, which was confirmed.

### Example 7. Heterologues Gene Expression and Protein Production in A. Woodii

### Primers

Pr55
gaGTCGACGCAGTATCTTAAAATTTTGTATAATAGGAATTGAAGTTAAA
TTAGATGCTAAAAATTTGTAATTAAGAAGGAGTGATTACATGTTACGTC
CTGTAGAAACC
Pr54
TTGCATGCTCATTGTTTGCCTCCC

### Strategy

The uidA (GUS) from E. coli BL21star(DE3) was amplified using gene specific primers, which included the sequence for constitutive promoters. Primer Pr55 includes the sequence from the Enterococcus faecalis Erythromycin resistance gene promoter, while primer Pr56 include the promoter sequence of the C. ljungdhalii PTA gene.

The amplified fragments were cloned into a plasmid capable of replicating in A. woodii. The replicative plasmid (pEP) carries the Erythromycin gene (described in Example 6) as well as the replicon pAMβ1. However, any other replicon suitable for A. woodii can be used. In this fashion two plasmids where generated, pEP55 carrying the uidA gene under control of the Enterococcus faecalis promoter and pEP56, carrying the uidA gene under control of the Clj promoter.

The generated plasmids were used to transform A. woodii. All following procedures were carried out under anaerobic conditions. A 10 ml culture was inoculate with A. woodii and grown to an OD600 of approximately 0.5. Cells were spun down in hungate tubes for 10 min at 4000 rpm at 4C and washed twice with 10 ml 10 ice-cold anaerobic 270 mM sucrose solution. The pellet was resuspended in 200 μL sucrose. 4 μl of plasmid was added to 40 μl cells, transferred to a 0.2-cm electroporation cuvette and kept on ice for 5 min. For electroporation the following settings were used: electric pulse of 10 kV, electric resistance of 400Ω and 25 μF. Following electroporation the cells were kept on ice for another 5 min. The transformed cells were recovered in media, incubated anaerobe for 6 h at 30 C and then transferred to 50 ml of medium containing the required antibiotic (20 μg/ml erythromycin). The cultures were incubated at 30C until growth was obtained. An aliquot of the cultures was plated on solid medium containing 20 μg/ml erythromycin. Single colonies were obtained after 5-7 days. For each transformation 2 independent colonies were picked and grown up in 10 ml cultures in the presence of erythromycin.

Functionality of uidA was established by restreaking the cultures on anaerobe selection media containing MUG (4-Methylumbelliferyl-β-D-glucopyranosiduronic acid, final concentration of 0.1 g/L) leading to a fluorescent product visible under UV-light as seen in FIG. 10.

The same strategy described above may be applied to the introduction any other heterologous gene (such as DERA, eutE etc) during construction of a 1,3-butanediol pathway and expressing it under a strong constitutive promoter on a replicative plasmid into A. woodii. The above described reporter gene uidA can be used to confirm the expression of any other gene, when cloned in an operon. Further, the expression of uidA can be used to determine promotor strength and hence promotor selection, as the efficiency of expression is related to fluorescence intensity. To enhance genetic stability heterologous genes may be introduced into the genome.

### Example 8. Generation of a Phosphotransacetylase (PTA) Mutant of Moorella thermoacetica ATCC39073 by Homologous Recombination

### Introduction

The M. thermoacetica ATCC39073 genome sequence has been published (Pierce et al., 2008) and is available at the NCBI with accession number NC_007644. The KEGG map of central carbon metabolism for M. thermoacetica ATCC39073 (http://www.genome.jp/kegg-bin/show_pathway?mta01200) was used to identify two putative phosphotransacetylases (PTAs), Moth_0864 and Moth_1181 (EC 2.3.1.8); which appear to be isoenzymes and are identified as being members of the PduL superfamily of bacterial propanediol utilisation proteins, based on sequence homology. Members of the phosphate acetyl/butaryl transferase (PTA/PTB) superfamily were not identified in the M. thermoacetica ATCC39073 genome; a BLASTP search of the partial PTA from Clostridium tyrobutyricum (Zhu et al., 2005) returned no significant alignments.

Construction of Moth_0864 and Moth_1181 Knockout Plasmids

### Construction of the Knockout Plasmid Backbone

The mobilisable shuttle vector pS797 is used as the backbone for construction of M. thermoacetica ATCC39073 knockout plasmids, since it already contains three of the desired genetic elements comprising the final construct; a pMB1 origin of replication for E. coli, an antibiotic selection marker (bla) and an RK4-derived conjugal origin of transfer (oriT) (Yakobson and Guiney, 1984). A thermostable (pJH₁-derived) kanamycin resistance gene for M. thermoacetica ATCC39073 has previously been described in the literature (Iwasaki et al., 2013), and was synthesised without further modification using the gene sequence from the S. faecalis pJH₁ kanamycin resistance gene (Genbank accession number V01547) fused to the native G3PDH promoter. Note that the knockout plasmid backbone does not include a replicon for M. thermoacetica ATCC39073, to ensure that kanamycin resistance can only be maintained in Moorella following a chromosome recombination event. Kanamycin-resistant transconjugants of M. thermoacetica ATC39073 are therefore all presumptive single crossover (SCO) chromosome mutants.

Primers APB57-65 were designed to generate knockout cassettes for Moth_0864 and Moth_1181.

| APB | | |
|---|---|---|
| # | Sequence (5′-3′) | Description |
| 57 | AGCTTTCGAGCGCGGA | 5′ phosphorylated. Universal |
| | AC | EMP R2 to splice knockout |
| | | cassettes into knockout |
| | | plasmids. |
| 58 | GAGTTCCATGTGGTCT | SOE F1 to clone upstream |
| | ACCATAC | region of chromosomal |
| | | homology for Moth_0864. |
| 59 | CATGGAGGTTAAGGCT | SOE R2; pair with APB58. |
| | GAGTTGACGATACACT | 945 bp product. Includes |
| | GTC | 13 bp overhang for assembly |
| | | with APB60/61 PCR product. |
| 60 | GTCAACTCAGCCTTAA | SOE 3F; to clone downstream |
| | CCTCCATGACGACCAG | region of chromosomal |
| | | homology for Moth_0864 |
| | | knockout. Includes 11 bp |
| | | overhang for assembly with |
| | | APB58/59 PCR product. |
| 61 | GACGAGCAAGGCAAGA | SOE 4R; pair with APB60. |
| | CCGGGATCCGACAGTA | 1015 bp product. Includes |
| | ACCGTAGGTACCTTCG | 25 bp 3′ overhang for EMP |
| | | splicing into plasmid |
| | | backbone. |
| 62 | CCAGTGATCTCTTTAT | SOE F1 to clone upstream |
| | CGACCTCC | region of chromosomal |
| | | homology for Moth_1181 |
| 63 | GGTGTGCATGTGCAAG | SOE F2, pair with APB62. |
| | GACACGCACCTTTTCT | 994 bp product. 3′ overhang |
| | AG | for SOE splicing with |
| | | APB64/65. |
| 64 | TGCGTGTCCTTGCACA | SOE 3F. Clone downstream |
| | TGCACACCGATGAGG | region of chromosomal |
| | | homology for Moth_1181. 5′ |
| | | overhang to splice with |
| | | APB62/63. |
| 65 | GACGAGCAAGGCAAGA | SOE 4R. Pair with APB64. |
| | CCGGGATCCGCTTCAA | 994 bp product. Includes |
| | CCCAAGCTTGTAGC | 25 bp 3′ overhang to EMP |
| | | PCR splice into knockout |
| | | plasmid backbone. |

Upstream and downstream regions of approximately 1 kb flanking a 282 bp and 176 bp internal region of Moth_0864 and Moth_1189, respectively were PCR-amplified from M. thermoacetica genomic DNA with compatible overhanging ends. The two flanking regions for each gene were then assembled into a single molecule of approximately 2 kb using SOE (splicing by overlap extension) PCR. These assembled knockout cassettes were independently spliced into the knockout plasmid backbone pDH160 by EMP PCR (Ulrich et al., 2012) to generate two new constructs; pDH177 and pDH180 (Moth_0864 and Moth_1189 knockout plasmids, respectively).

Generation of Independent SCO Mutant Strains of Moth_0864 and Moth_1189 in M. thermoacetica ATCC39073

Knockout plasmids are used to independently transform E. coli conjugal donor strain S17-1 and resulting strains maintained on selective agar media containing 100 μg/ml carbenicillin. For each gene knockout, biomass equivalent to a 10 μl inoculation “loopful” from overnight growth of the conjugal donor strain and the conjugal recipient strain (the latter being wild-type M. thermoacetica ATCC39073 grown on brain-heart infusion agar (BHIA; Oxoid) supplemented with 2% (w/v) fructose (BHIAF) and incubated at 55° C.) are emulsified and spread onto BHIA. The conjugation mix is incubated for 8 hours at 37° C. and is then re-suspended in 1 ml of pre-reduced ATCC medium 1754 using a sterile spreader. The emulsified conjugation mix is diluted 10⁻¹ to 10⁻⁶ in ATCC medium 1754 and 200 μl of each dilution is spread onto selective agar (BHIAF plus kanamycin 150 μg/ml) and incubated at 55° C. in anaerobic jars. Transconjugant Moorella colonies (presumptive SCOs) are typically recovered within 8-10 days. This is believed to be the first account of genetic transformation of Moorella sp. using conjugation.

Generation of Double Crossover (DCO) Stable Mutants of Moth_0864 and Moth_1189 in M. thermoacetica ATCC39073

The following method can be used to isolate chromosomal deletion mutants, generated from SCOs by homologous recombination following sequential passage. Single, isolated transconjugant colonies of M. thermoacetica ATCC39073 can be used to independently inoculate 20 mL aliquots of pre-reduced ATCC medium 1754 in sealed Hungate tubes and are incubated for at least 24 hours, until turbid; this is passage 1. Following incubation, 4 mL of passaged culture is added to 4 mL of 50% (v/v) pre-reduced glycerol in a sealed serum bottle and is stored at −80° C. In addition, 100 μL of passaged culture is diluted 10⁻¹ to 10⁻⁶ in ATCC medium 1754 and 200 μl of each dilution are spread onto selective agar (BHIAF plus kanamycin 150 μg/ml) and incubated at 55° C. in anaerobic jars to isolate single colonies. Finally, 200 μl of passaged culture is used to inoculate a 20 mL aliquot of pre-reduced ATCC medium 1754 in a sealed Hungate tube and is incubated for at least 24 hours, until turbid (passage 2). Passaging of SCOs proceeds until kanamycin-sensitive colonies are isolated (see below).

Single colonies isolated from each passage (approximately 100) are replica-plated onto BHIAF with and without 150 μg/ml kanamycin and are incubated at 55° C. Kanamycin-sensitive colonies are presumptive double-crossover mutants (i.e the knockout plasmid has been lost following a second recombination event). Genomic DNA is prepared from presumptive DCO mutants and the target gene is PCR-cloned and sequenced to check for the designed deletion mutation.

### REFERENCES FOR EXAMPLE 8

Iwasaki, Y., Kita, A., Sakai, S., Takaoka, K., Yano, S., Tajima, T., Kato, J., Nishio, N.,
Murakami, K., and Nakashimada, Y. (2013). Engineering of a functional thermostable kanamycin resistance marker for use in Moorella thermoacetica ATCC39073. FEMS Microbiol. Lett. 343, 8-12.
Pierce, E., Xie, G., Barabote, R. D., Saunders, E., Han, C. S., Detter, J. C., Richardson, P., Brettin, T. S., Das, A., Ljungdahl, L. G., et al. (2008). The complete genome sequence of Moorella thermoacetica (f. Clostridium thermoaceticum). Environ. Microbiol. 10, 2550-2573.
Ulrich, A., Andersen, K. R., and Schwartz, T. U. (2012). Exponential Megapriming PCR (EMP) Cloning—Seamless DNA Insertion into Any Target Plasmid without Sequence Constraints. PLoS ONE 7, e53360.
Yakobson, E. A., and Guiney, D. G. (1984). Conjugal transfer of bacterial chromosomes mediated by the RK2 plasmid transfer origin cloned into transposon Tn5. J. Bacteriol. 160, 451-453.
Zhu, Y., Liu, X., and Yang, S.-T. (2005). Construction and characterization of pta gene-deleted mutant of Clostridium tyrobutyricum for enhanced butyric acid fermentation. Biotechnol. Bioeng. 90, 154-166.

### Example 9. Enzymes for the Reduction of 3-Hydroxybutanal to 1,3-Butanediol

### Introduction

This example describes the ability for selected reductases to demonstrate a preference for a C4 aldehyde (model substrate butanal and target 3-hydroxybutanal) relative to a C2 aldehyde (acetaldehyde) as discussed in Example 3; GOX1615 from Gluconobacter oxydans, BdhB from Clostridium acetobutylicum and GRE2 from Saccharomyces cerevisiae were selected for demonstration of this required principle. There follows a description of cloning, purification and enzyme assay for these three selected enzymes.

### Gene and Protein Information

| | UniProt | | | Size of his |
|---|---|---|---|---|
| Gene Name | entry | NCBI ID | Organism | tagged protein |
| GOX1615 | Q5FQJ0 | 3248904 | Gluconobacter | 39376.7 |
| | | | oxydans | |
| BdhB | Q04945 | 1119480 | Clostridium | 45450 |
| CA_C3298 | | | acetobutylicum | |
| GRE2 | Q12068 | 854014 | Saccharomyces | |
| YOL151W | | | cerevisiae | |
| | | | (strain ATCC | |
| | | | 204508/S288c | |

### a) GOX1615

### Construction of a GOX1615 Expression Plasmid

Primers Pr89 (5′-GCCATATGGCATCCGACACCATCC) and Pr90 (5′-CCGGATCCTCAGTCCCGTGCC) were used to amplify the G. oxydans GOX1615 gene; which had previously been obtained by commercial DNA synthesis and delivered on a plasmid. The amplicon was cloned into pET3a and pET14b (Novagen); with the latter construct adding an N-terminal 6-His tag to the GOX1615 coding sequence in order to facilitate purification of the enzyme by nickel-affinity chromatography.

PCR was performed using Q5 proofreading DNA polymerase (New England Biolabs) following the manufacturer's protocol and using an annealing temperature of 55° C. The resulting PCR product (1008 bp) was purified by gel extraction, and was then digested using NdeI and BamHI restriction endonucleases (New England Biolabs). Following heat inactivation of the restriction enzymes (manufacturer's protocol), the digested PCR product was ligated into pET14b and pET3a and an aliquot of the ligation mix was used to transform E. coli DH10B. Transformants were screened for presence of the GOX16515 gene by colony PCR using T7 forward and reverse primers (using a Taq polymerase with annealing at 55° C.). Two positive clones from each transformation were picked for plasmid DNA extraction and the correct constructs further confirmed by restriction digest. The positive clones were stored in 15% glycerol at −80° C. (pET14b-GOX1615: pDH358 and pDH359; pET3a-GOX1615 pDH351, pDH353). Expression plasmid pDH358 was subsequently confirmed by sequencing using primers pET3a-F and pET3a-R.

### Expression and Purification of GOX1615

Plasmid pDH358 (pET14b-GOX) was used to transform E. coli BL21 Star (DE3) with the resulting strain (DH369) stored in 15% glycerol at −80° C. A single colony of DH369, and vector control strain DH228, were inoculated into 5 mL auto inducing medium (per litre: 6 g Na₂HPO₄, 3 g KH₂PO₄, 5 g Yeast extract, 5 g NaCl with 10 mL 60% v/v glycerol, 5 mL 10% w/v glucose, 25 mL 8% w/v lactose filter sterilised and added post autoclaving; Studier, F. W. 2005) in 50 mL tubes and the cultures grown overnight at 37° C. with continuous shaking at 225 rpm. 1 mL aliquots of cells were harvested by centrifugation and the cell pellets resuspended in 200 μl of Bugbuster (Novagen). After incubation at room temperature for 20 minutes, the mix was centrifuged for 5 min at 14000× g and the supernatant retained. The resulting pellet was resupended in 200 μl 50 mM Tris-HCl pH 7.0. Supernatant and pellet samples were mixed 1:1 was 2×SDS buffer, boiled for 10 min, centrifuged for 2 min at 14000×g and subsequently 5 μl of each sample was loaded on a 12% gel SDS PAGE gel to confirm presence of GOX1615 protein.

For purification of His-tagged GOX1615, 1 mL of an overnight DH369 culture grown in LB containing 100 μg/mL carbenicillin was used to inoculate 100 mL of the same medium. Cells were grown at 37° C. to an OD600 nm of 0.6-0.9. Gene expression was induced with 0.4 mM IPTG and cells incubated overnight with shaking at 200 rpm at 18° C.

The culture was harvested by centrifugation and samples were maintained at 4° C. for subsequent steps. The pellet was resuspended in 5 mL binding buffer (50 mM Na-phosphate pH 8.0, 0.5 M NaCl, 5 mM Imidazole) and sonicated on ice: 5×30 sec; amplitude 10% with a 30 sec break between pulses. The lysed cell suspension was clarified by centrifugation at 14000×g for 15 mins, 4° C. and GOX1615 purified from the cleared cell lysate by affinity purification using a HisTrap™ HP 5 mL column and AKTA start chromatography (GE Healthcare Life Sciences) system following manufacturers protocols. Purified protein was stored at −80° C.

Assay of GOX1615 Reductase Activity with C2 and C4 Substrates

Assays were performed in a volume of 1 mL directly in 1.5 mL UV cuvettes. Consumption of NAD(P)H was measured at 340 nm. The reaction was started by addition of 100 μL of substrate solution and measured over 2-5 min.

3-Hydroxybutanal (technical grade) was purchased from BOC. Activities are therefore minimal activities when in the linear range as the standard was not pure.

The results shown below indicate the desired preference GOX1615 displays for a C4 aldehyde including 3-hydroxybutanal compared with activity towards acetaldehyde as substrate:

| | | | |
|---|---|---|---|
| Concentration | 3-hydroxybutanal | Butanal | Acetaldehyde |

| | |
|---|---|
| Substrate (mM) | (units) |

GOX1615, 0.15 mM NADPH, 50 mM Na-phosphate pH 7.0, 25° C.

| | | | |
|---|---|---|---|
| 1 | 0.1 | 9.63 | 0.006 |
| 5 | 2.49 | 17.7 | |
| 10 | 3.75 | 23.3 | 0.12 |
| 20 | 5.28 | | |

GOX1615, 0.15 mM NADH, 50 mM Na-phosphate pH 7.0, 25° C.

| | |
|---|---|
| 10 | 0.37 |

1 unit = 1 μmol/min/mg

### b) BdhB

### Construction of BdhB Expression Plasmids

The bdhB gene of Clostridium acetobutylicum was obtained by commercial DNA synthesis and was independently spliced into expression vectors pET3a and pET14b; in-frame with the 3′ sequence encoding a 6-His tag of the latter, using EMP PCR. Sequenced clones pDH365, pDH366 (pET14b-BdhB) and pDH380, pDH381 (pET3a-BdhB) were used to transform E. coli DH10B and were stored in 15% Glycerol at −80 C.

### Expression and Purification of BdhB

Plasmid pDH365 (pET14b-BdhB, MP1) was used to transform BL21*(DE3). The generated culture was stored in LB containing 15% glycerol at −80° C. (DH372). A 400 mL auto-inducing media (Foremedia) culture was inoculated from glycerol stock and grown for 20-24 h at 30° C., 250 rpm shaking. The culture was spun down (4000 rpm, 30 min, 4=C), the pellet was washed twice with 10 mM sodium phosphate buffer pH 7.0 and resuspended in 5 mL Binding buffer with ZnSO4 and DTT (10 mM Na-phosphate pH 7.0, 5 mM Imidazole, 0.1 mM ZnSO₄, 1 mM DTT and protease inhibitors). Cells were lysed by glass beads (four cycles of 20 s, 5.5 m/s, two minutes on ice between each cycle) cell lysate was cleared by centrifugation at 4000 rpm, 15 min, 4° C. and purified using a 5 ml HisTrap column and AKTA 900 system as described previously for purification of GOX1615. All procedures were carried out under anaerobic conditions

Assay of BdhB Reductase with C2 and C4 Substrates

The activity of BdhB against acetaldehyde and butanal was measured at 25° C. at 1 ml reaction volume under anaerobic conditions. Enzyme assays were performed in 50 mM MES buffer pH 6.5 containing 1 mM DTT and 0.1 mM ZnSO4. Reactions were carried out in disposable UV cuvette sealed with a rubber stopper. Consumption of NAD(P)H was measured at 340 nm. The reaction was started by adding 100 μl of the substrate. The linear reaction was measured over a range of 10 min. The following data were obtained.

| | | |
|---|---|---|
| Concentration | Butanal | Acetaldehyde |

| | |
|---|---|
| Substrate (mM) | (units) |

BdhB, 0.15 mM NADH, 50 mM MES pH 6.5, 0.1 mM ZnSO₄ 25° C.

| | | |
|---|---|---|
| 1 | 0.03 | No measured |
| | | activity |
| 5 | 0.06 | No measured |
| | | activity |

BdhB, 0.15 mM NADPH, 50 mM MES pH 6.5, 0.1 mM ZnZnSO₄ 25° C.

| | | |
|---|---|---|
| 1 | 0.14 | No measured |
| | | activity |
| 5 | 0.27 | No measured |
| | | activity |

1 unit = 1 μmol/min/mg

In a separate experiment 3-hydroxybutanal was also shown to be a substrate. At 5 mM 3-hydroxybutanal with co factor NADH the rate was 0.012 μmol/min/mg protein

### c) GRE2

### Construction of a GRE2 Expression Plasmid

GRE2 was PCR-amplified from Saccharomyces cerevisiae genomic DNA using a proofreading DNA polymerase and primer pair Pr91 (5′-GCCATATGTCAGTTTTCGTTTCAGG) and Pr92 (5′-CGGATCCTTATATTCTGCCCTC). The 1038 bp PCR product was purified by gel extraction and was then restriction-cloned into expression plasmids pET14b and pET3a via 5′ NdeI and 3′ BamHI enzyme cleavage sites; a method well-known in the art. The resulting ligation mixes were used to independently transform aliquots of chemically-competent E. coli DH10B. Successful clones for each ligation were identified by colony PCR and further confirmed by restriction analysis of plasmid minipreps. Resulting plasmids were assigned the following IDs: pET14b-GRE2: pDH360; pET3a-GRE2: pDH376.

### Expression and Purification of GRE2

Plasmid pDH360 was used to transform E. coli BL21 Star (DE3). For protein production, the resulting strain (DH370) was used to inoculate an LB medium containing 100 μg/mL carbenicillin and incubated at 37° C. to an OD600 of 0.6-0.9. GRE2 expression was induced with 0.4 mM IPTG and incubation at 18° C. with shaking at 200 rpm for 18 hours.

Induced bacteria were recovered by centrifugation and protein purification was carried out as follows: bacteria were resuspended in 5 ml binding buffer (50 mM Na-phosphate pH 8.0, 0.5 M NaCl, 5 mM Imidazole) and sonicated on ice for 5×30 sec, amplitude 10% with a 30 sec break between pulses. Lysed bacteria were recovered by centrifugation at 15000 rpm and 4° C. An aliquot of the supernatant was kept for analysis by SDS PAGE before the remaining supernatant was loaded on a 3 or 5 mL nickel affinity column (Qiagen, NTA), which had been equilibrated with 10 column volumes of binding buffer. The flow-through was collected for analysis by SDS PAGE. Unbound protein was washed from the column with 15 ml of binding buffer and 15 ml of wash buffer (50 mM Na-phosphate pH 8.0, 0.5 M NaCl, 100 mM Imidazole). Again the flow-through was collected for SDS gel analysis. Finally bound protein was eluted with 10 ml of elution buffer (50 mM Na-phosphate pH 8.0, 0.5 M NaCl, 400 mM Imidazole). The presence of protein in eluted fractions was rapidly confirmed by Bradford assay and were then further analysed by SDS PAGE for the presence of GRE2. Enzyme-containing fractions were buffer-exchanged to 50 mM Na-phosphate pH 7.0 immediately after purification using PD10 columns. Purified enzyme was stored at 4° C. until assay.

Assay of GRE2 Reductase Activity with C2 and C4 Substrates

The activity of GRE2 against acetaldehyde and butanal was studied. Reactions were carried out 25° C. at 1 mL reaction volume. Enzyme assays were performed in disposable UV cuvette sealed with a rubber stopper. Consumption of NAD(P)H was measured at 340 nm. The reaction was started by adding 100 μl of the substrate. The linear reaction was measured over a range of 2-5 min. The following data were obtained and show the required preference for the longer chain aldehyde butanal relative to acetaldehyde. As shown for the examples above, GRE2 would be expected to be active on 3-hydroxybutanal.

GRE2, 0.15 mM NAD(P)H, 50 mM Na-phosphate pH 7.0, 25 C.

| | |
|---|---|
| NADPH | NADH |

| Butanal (mM) | units | Butanal (mM) | units |
|---|---|---|---|
| 1 | 5.5 | 1 | 0.14 |
| 2 | 13.8 | 5 | 0.63 |
| 5 | 24 | 10 | 0.84 |
| 10 | 21.6 | 20 | 1.35 |

NADPH

| | |
|---|---|
| Acetaldehyde | NADH |

| (mM) | units | Acetaldehyde (mM) | units |
|---|---|---|---|
| 10 | 0.57 | 5 | 0.029 |
| 40 | 1.64 | 10 | 0.032 |

1 unit = 1 μmol/min/mg

### REFERENCES FOR EXAMPLE 9

Studier, F. W. Protein production by auto-induction in high density shaking cultures. Protein Expr Purif. 2005 May; 41(1):207-34.

### Example 10. Production of 1,3-Butanediol from Acetyl CoA (Route 2, FIG. 3)

Described below is an in vitro example of a pathway where DERA is supplied with acetaldehyde from an acetaldehyde dehydrogenase and where the DERA product 3-hydroxybutanal is reduced to 1,3-butanediol using GOX1615 reductase. Further methodology is described in Example 11 below. These data demonstrate how DERA can be supplied acetaldehyde from a preceding pathway enzyme to effect synthesis of hydroxybutanal and a downstream product (here: 1,3-butanediol).

| | UniProt | | | Size of his |
|---|---|---|---|---|
| Gene Name | entry | NCBI ID | Organism | tagged protein |
| eutE | P41793 | 1253985 | Salmonella | 50 KDa |
| | | | enterica subsp. | |
| | | | enterica | |
| | | | serovar | |
| | | | Typhimurium | |
| | | | str. LT2 | |

### Expression and Preparation of Lysates Containing EutE

E. coli BL21 Star (DE3) cells bearing either an empty pET3a vector (DH228) or a DERA:EutE fusion (DH357; Example 11 below) were inoculated as a seed culture in 5 ml LB medium (10 g/l tryptone, 5 g/l yeast extract, 10 g/L NaCl) containing 100 μg/ml carbenicillin. After overnight growth at 37° C., cultures were diluted to OD590 nm 0.1 and grown at 37° C. in 50 ml of the same medium to an OD590 nm of 0.4 to 0.6. Protein expression was then induced by adding 0.4 mM IPTG, followed by incubation at 18° C. overnight with shaking.

Following overnight growth, induced bacteria were recovered by centrifugation at 4,000 rpm for 10 min at 4° C. The pellet was then washed twice with 10 mM sodium phosphate buffer, pH 7.0 and resuspended in 2 ml of lysis buffer (10 mM sodium phosphate buffer pH 7.0 containing 1 mM DTT and protease inhibitor cocktail (SigmaFast, Sigma S8820)). Cells were lysed by sonication as described previously. The resulting lysate was clarified by a centrifugation step at 15,000×g for 5 min at 4° C. and the supernatant was recovered.

Production of 1,3-butanediol was carried out for an arbitrary 120 hours at 25° C. using the reagents shown below with a lysate volume of 1/10th the assay volume.

Reagents and Concentrations Used for the Production of 1,3-BDO from Acetyl-CoA.

Cofactor recycle was achieved using glucose and glucose dehydrogenase (GDH)

| Reagent | Concentration in assay | |
|---|---|---|
| Acetyl-coA | 10 | mM |
| NADH | 0.15 | mM |
| NADPH | 0.15 | mM |
| GDH | 0.1 | mg/ml |
| GOX1615 | 0.011 | mg/ml |
| Glucose | 10 | mM |
| EcDERA (Sigma 91252), added where | 12 mg/ml (3.6 units/mg, Sigma) | |
| indicated | | |
| Buffer | qs 250 | μl |

A control reaction comprised 10 mM acetaldehyde, 0.15 mM NADH, 0.15 mM NADPH, 0.1 mg/mL GDH and 0.011 mg/mL GOX1615 in a 10 mM sodium phosphate buffer, pH 7.0 without addition of DERA or lysate. This control confirmed that no 1,3-BDO was produced via abiotic chemical condensation of acetaldehyde to 3-hydroxybutanal Detection of 1,3-BDO, ethanol and acetaldehyde was carried out using HPLC (Phenomenex Rezex OA column organic acid H+300×7.8 mm).

### Results

| | | | |
|---|---|---|---|
| | [Acetaldehyde] | [1,3 Butanediol] | [Ethanol] |
| Incubation Conditions | mM | mM | mM |

DH228 (Empty Vector no eutE) + EcDERA

| | | | |
|---|---|---|---|
| −GOX1615 | ND | ND | ND |
| +GOX1615 | ND | ND | ND |

DH357 (eutE-EcDERA fusion) + EcDERA

| | | | |
|---|---|---|---|
| −GOX1615 | ND | ND | ND |
| +GOX1615 | ND | 0.31 | ND |

ND = None detected

The control reaction (containing 10 mM acetaldehyde alone) exhibited no detectable 1,3-butanediol.

1,3-Butanediol was confirmed by LC/mass spectrometry. Representative mass spectrometry data are shown in FIG. 16.

### Example 11. Construction of a EutE:DERA Fusion Protein

### Introduction

A fusion of heterologous pyruvate dehydrogenase (PDC) and alcohol dehydrogenase (ADHE), expressed in E. coli has been previously shown to exhibit improved ethanol production when compared to individually expressed enzymes alone, despite the fusion enzyme having a 20-fold less specific activity for ADH (Lewicka et al 2014). Substrate channeling of acetaldehyde (a cytotoxic, volatile intermediate of relevance to this invention) was attributed to the observed improvement in ethanol titre.

A functional enzyme fusion of DERA and an acetaldehyde dehydrogenase (e.g eutE), or DERA and pyruvate decarboxylase or DERA and an enzyme capable of acetate reduction for example, would be expected to work in a comparable way to improve the conversion of acetyl-CoA to 3-hydroxybutanal; (or pyruvate or acetate to 3-hydroxybutanal) either by substrate channeling of the acetaldehyde intermediate, or by locally increasing the substrate concentration around the DERA active site. In principle any component of a complete DERA pathway could be introduced as a fusion protein to optimise pathway performance.

Proteins comprising a biosynthetic pathway may also be linked by other approaches whereby the enzymes are not fused, but are retained in close proximity. For example, the localisation to a bacterial microcompartment by the use of an N-terminal targeting peptide in order to generate an “ethanol bioreactor” within the cell (Lawrence et al 2014); or the potential for use of bacterial scaffoldins to position proteins into a complex (Ding et al 2003). These techniques could equally be applied to the current invention.

### Construction of a EutE:DERA Fusion

An enzyme fusion comprising the E. coli K12 DERA (GenBank: CAA26974.1) and EutE (Salmonella typhimurium LT2; GenBank: AAL21357.1) was constructed by removing the corresponding start and stop codons from an existing polycistronic expression operon using inverse PCR without the addition of a linker, with the resulting fusion enzyme found to be functional. The following method for creating enzyme fusions may be applied to one or more of the enzymes comprising a metabolic pathway containing DERA for the purpose of the synthesis of 1,3-butanediol or other chemicals.

A divergent primer pair APB142F (5′-AATCAACAGGATATTGAACAGGTGGTG; 5′ phosphorylated) and APB143R (5′-GTAGCTGCTGGCGCTCTTAC) were designed to remove the intergenic region between adjacent DERA and EutE genes (including the stop codon of the DERA coding sequence and the start codon of the downstream EutE coding sequence in plasmid pDH291, pET3a-DERA-EutE-GOX1615) by inverse PCR; such that the two coding sequences would be fused into one continuous open reading frame when the PCR product (comprising the entire expression plasmid) was re-ligated. The linear 7.8 kb APB142/APB143 inverse PCR product was purified, ligated and used to transform chemically-competent E. coli JM109. Two carbenicillin-resistant transformants were subcultured on selective media and assigned strain IDs DH337 and DH338, respectively. Strains DH356 (E. coli BL21 Star (DE3)/pDH337), DH357 (E. coli BL21 Star (DE3)/pDH338), DH301 (E. coli BL21 Star (DE3)/pDH291) and DH228 (E. coli BL21 Star/pET3a; negative control) were used to independently inoculate 6 mL of auto-inducing media (Studier 2005) with 100 μg/L carbenicillin and were incubated overnight (16-18 h) at 37° C., 225 rpm. Following incubation, biomass was recovered by centrifugation and lysed at 4×5.5 m/s in a FastPrep bead beater, using 0.1 mM acid-washed glass beads. The soluble fraction (supernatant) was recovered by centrifugation (13.4 krpm at 4° C. in a bench-top centrifuge) and proteins resolved by 10% SDS PAGE to confirm expression of the 76.76 KDa DERAE-EutE fusion protein in both strains DH356 and DH357.

The fusion protein was expressed as described in Example 10. The fusion protein was confirmed to be active with respect to both acetaldehyde dehydrogenase activity (eutE) and deoxyribose-5-P-phosphate aldolase (DERA) activity. Assays were carried out as described in Example 13 using an alcohol dehydrogenase linked assay to detect the product acetaldehyde from either acetyl CoA or deoxyribose-5-P-phosphate respectively. Measured activities were 4.2 μmol/min/mg and 5 μmol/min/mg.

### REFERENCES FOR EXAMPLE 11

Ding S Y, Lamed R, Bayer E A, Himmel M E. The bacterial scaffoldin: structure, function and potential applications in the nanosciences. Genet Eng (N Y). 2003; 25:209-25.
Lawrence A D, Frank S, Newnham S, Lee M J, Brown I R, Xue W F, Rowe M L, Mulvihill D P, Prentice M B, Howard M J, Warren M J. Solution structure of a bacterial microcompartment targeting peptide and its application in the construction of an ethanol bioreactor. ACS Synth Biol. 2014 Jul. 18; 3(7):454-65.
Lewicka A J, Lyczakowski J J, Blackhurst G, Pashkuleva C, Rothschild-Mancinelli K, Tautvai\check{s}as D, Thornton H, Villanueva H, Xiao W, Slikas J, Horsfall L, Elfick A, French C. Fusion of pyruvate decarboxylase and alcohol dehydrogenase increases ethanol production in Escherichia coli. ACS Synth Biol. 2014 Dec. 19; 3(12):976-8

### Example 12. Production of 1,3-Butanediol from Pyruvate Using Pyruvate Decarboxylase (Isolated Enzymes). Route 4, FIG. 3

### Introduction

Described below are in vitro examples of a pathway where DERA is supplied acetaldehyde from pyruvate decarboxylase and where the DERA product 3-hydroxybutanal is reduced to 1,3-butanediol using GOX1615 reductase or bdhB dehydrogenase. This work provides detailed data regarding the production of 1,3-butanediol from pyruvate. Increasing pyruvate concentration provides increasing amounts of acetaldehyde supply to the DERA enzyme.

These data demonstrate how DERA can be supplied acetaldehyde from a preceding pathway enzyme to effect synthesis of hydroxybutanal and a downstream product (here: 1,3-butanediol).

1,3-Butanediol Production with Increasing Pyruvate Concentration

Acetaldehyde was supplied to the enzyme system (1 ml) via decarboxylation of pyruvate using S. cerevisiae pyruvate decarboxylase (PDC1) 0.5 U/ml (Sigma P9474). Pyruvate was added at 5, 10, 15, 20, 30 and 50 mM. The reaction also contained 12 mg E. coli deoxyribose-5-P aldolase (DERA, 3.6 units/mg, Sigma) and 0.011 mg/ml of purified GOX1615). Recycling of the cofactor NADPH (0.15 mM) was provided by glucose dehydrogenase (GDH) from Pseudomonas sp. (Sigma 19359) added at a final concentration of 0.1 mg/ml. The concentration of reactants and products were monitored by HPLC (Phenomenex Rezex OA column organic acid H+300×7.8 mm).

The reaction was incubated at 25° C. for an arbitrary 96 hours.

1,3-Butanediol Production from Increasing Concentration of Pyruvate.

| Initial | | | [Acetaldehyde] |
|---|---|---|---|
| [Pyruvate] mM | [1,3 Butanediol] mM | [Ethanol] mM | mM |
| | Test Reaction (PDC + DERA + GOX1615) | | |
| 5 | 0.65 | ND | ND |
| 10 | 1.28 | ND | ND |
| 15 | 1.84 | ND | 3.06 |
| 20 | 2.28 | 1.18 | 3.17 |
| 30 | 2.56 | 1.60 | 7.64 |
| 50 | 4.17 | 1.53 | 14.86 |
| | Control PDC + GOX1615 (No DERA) | | |
| 5 | ND | 1.01 | 3.51 |
| 10 | ND | 2.19 | 7.23 |
| 15 | ND | 2.84 | 9.86 |
| 20 | ND | 3.53 | 13.07 |
| 30 | ND | 5.16 | 22.81 |
| 50 | ND | 4.70 | 33.61 |
| | Control PDC + DERA (No GOX 1615) | | |
| 5 | ND | ND | 1.37 |
| 10 | ND | ND | 1.73 |
| 15 | ND | ND | 2.34 |
| 20 | ND | ND | 3.66 |
| 30 | ND | ND | 7.17 |
| 50 | ND | ND | 14.70 |

An example using bdhB dehydrogenase is given below. The same method was used except that pyruvate was provided at 30 mM and bdhB added at 0.07 mg/ml

| 96 Hour | BdhB Test reaction | | |
|---|---|---|---|
| Incubations | (PDC + DERA + GOX1615) | | |
| Initial [Pyruvate] | | [1,3 Butanediol] | [Ethanol] |
| mM | [Acetaldehyde] mM | mM | mM |
| 5 | 1.35 | ND | 1.73 |
| 30 | 9.34 | 0.4 | 2.46 |
| | Control PDC + BdhB (No DERA) | | |
| Initial [Pyruvate] | | [1,3 Butanediol] | [Ethanol] |
| mM | [Acetaldehyde] mM | mM | mM |
| 5 | 4.64 | ND | 1.14 |
| 30 | 32.87 | ND | 1.45 |

ND Not detected

1,3-butanediol was confirmed in all cases by LC/mass spectrometry. Representative mass spectrometry data is shown in FIG. 16.

1,3-Butanediol Production from 5 mM or 30 mM Pyruvate at Different DERA Concentrations

Reactions containing 5 mM or 30 mM pyruvate as substrate and GDH cofactor recycling for GOX1615 were set up as above except the amount of E. coli DERA (3.6 units/mg, Sigma) was varied at, 12, 6, 3, 1.5, 0.75 mg. Reactants and products were monitored as above.

The reaction was incubated at 25° C. for an arbitrary 96 hours.

### 1,3-Butanediol Production

Pyruvate supplied at 5 mM

| | [Acetaldehyde] | [1,3 Butanediol] | [Ethanol] |
|---|---|---|---|
| DERA mg/ml | mM | mM | mM |
| 0.75 | 4.07 | 0.17 | ND |
| 1.5 | 3.26 | 0.24 | ND |
| 3 | 1.89 | 0.36 | ND |
| 6 | 1.64 | 0.43 | ND |
| 12 | 1.35 | 0.53 | ND |

Pyruvate supplied at 30 mM

| | [Acetaldehyde] | [1,3 Butanediol] | [Ethanol] |
|---|---|---|---|
| DERA mg/ml | mM | mM | mM |
| 0.75 | 31.32 | 0.47 | 2.52 |
| 1.5 | 29.62 | 0.80 | 2.11 |
| 3 | 26.62 | 1.36 | 1.69 |
| 6 | 22.01 | 1.93 | 1.71 |
| 12 | 14.76 | 2.48 | 1.25 |

Control PDC and GOX1615 no DERA

| [Pyruvate] | [Acetaldehyde] | [1,3 Butanediol] | [Ethanol] |
|---|---|---|---|
| mM | mM | mM | mM |
| 5 | 4.61 | ND | ND |
| 30 | 30.68 | ND | 2.14 |

ND = none detected

1,3-butanediol was confirmed in all cases by LC/mass spectrometry. A representative mass spectrum is shown in FIG. 16.

Overall, the production of ethanol as a by-product can be improved either by improvement of the DERA enzyme (e.g evolution for better kinetics) or further evolution of a selective reductase towards reduction of 1,3-butanediol.

### Example 13. Production of 1,3-Butanediol from Pyruvate Using Selected DERAs from Different Microbial Sources

### Introduction

This example demonstrates that DERA's from a range of microorganisms can be suitable for condensation of two molecules of acetaldehyde to the intermediate 3-hydroxybutanal as part of a novel, in vivo unnatural metabolic pathway.

### Target Gene and Protein Information

| | | | | Size of |
|---|---|---|---|---|
| Name | UniProt entry | NCBI ID | Source organism | protein |
| EcDERA | P0A6L0 | 948902 | Escherichia coli | 27.73 KDa |
| AwDERA | H6LF13 | WP_014354523.1 | Acetobacterium | 23.84 KDa |
| | | | woodii | |
| PaDERA | Q8ZXK7 | 1465578 | Pyrobaculum | 24.54 KDa |
| | | | aerophilum | |
| GtDERA | A4IR26 | WP_008879914.1 | Geobacillus | 23.3 KDa |
| | | | thermodenitrificans | |
| | | | NG80-2 | |

Isolation and Sequencing of a Geobacillus thermodenitrificans Strain NG80-2 DERA Homolog from Geobacillus thermodenitrificans Strain K1041

A homolog of the gene GTNG_2435 from G. thermodenitrificans strain NG80-2 (for which there is a published genome sequence) was identified, PCR cloned and sequenced from G. thermodenitrificans K1041. PCR primers APB106F (5′-ATGACGGTGAATATTGCTAAAATGATCG) and APB107R (5′-TTAATAGTCAGCGCCGCCGGTTTG) were designed based on the GNTG_2435 sequence as a template, and were used along with Q5 High-Fidelity DNA Polymerase (New England Biolabs) and the manufacturer's recommended PCR reaction conditions to PCR-clone an approximate 672 bp product from G. thermodenitrificans K1041 genomic DNA; confirmed by agarose gel electrophoresis. The PCR product was directly ligated into cloning vector pJET1.2 using the CloneJET PCR Cloning Kit (Thermo Scientific) according to the manufacturer's protocol for blunt-ended PCR products, with the resulting ligation mix used to transform chemically-competent E. coli DH10B, with transformants selected by incubation on Luria Agar (LA; Sigma) plus carbenicillin at a concentration of 100 μg/mL. Transformant E. coli DH10B colonies recovered following 16 hours incubation at 37° C. were replica-plated onto LA plus carbenicillin 100 μg/mL and checked for the presence of cloned APB106F/APB107R PCR product using primer pair APB106F and pJET1.2 reverse sequencing primer (Thermo Scientific) with DreamTaq DNA Polymerase (Thermo Scientific) in a colony PCR reaction; with the presence of an approximate 729 bp product in PCR-positive transformant colonies confirmed by agarose gel electrophoresis. Two of these were stored with strain IDs DH208 and DH209, respectively. Plasmids were isolated from these strains by alkaline lysis and were sequenced using pJET1.2 forward and reverse sequencing primers (Thermo Scientific) to derive the sequence of G. thermodenitrificans strain K1041 GNTG_2435 homolog. This gene was subsequently identified as encoding a putative DERA by both nucleotide sequence homology with GNTG_2435 and identification of conserved domains within the translated primary amino acid sequence using the NCBI BLAST web server. The sequence of the K1041 homolog is reproduced below:

ATGACGGTGAATATTGCTAAAATGATCGATCATACGTTGCTTAAGCC
AGAAGCGACGGAAGAGCAAATCATTCAACTATGCGACGAAGCAAAGC
AACACGGCTTCGCCTCGGTGTGCGTCAACCCAGCGTGGGTGAAAACA
GCGGCACGCGAGCTTTCCGACACTGATGTCCGCGTCTGCACGGTCAT
CGGCTTTCCGCTTGGGGCGACGACGCCGGAAACAAAGGCGTTTGAAA
CGAACAACGCTATCGAAAACGGCGCCCGCGAAGTCGATATGGTAATC
AACATCGGCGCGTTAAAAAGTGGTAACGATGAACTCGTTGAGCGCGA
CATTCGTGCGGTTGTTGAGGCGGCGTCCGGGAAAGCGCTTGTGAAAG
TGATCATCGAAACGGCCTTGTTGACTGATGAGGAAAAAGTGCGCGCC
TGCCAATTGGCGGTGAAAGCGGGCGCCGATTACGTAAAAACGTCGAC
CGGATTCTCAGGCGGCGGAGCGACGGTCGAAGACGTGGCGCTGATGC
GCCGGACAGTTGGCGATAAAGCAGGTGTCAAAGCCTCAGGAGGCGTC
CGCGACCGAAAAACAGCCGAAGCGATGATTGAAGCTGGGGCCACGCG
CATTGGGACGAGCTCCGGGGTGGCGATCGTCAGCGGCCAAACCGGCG
GCGCTGACTATTAA

Construction of Expression Vectors Containing E. coli, A. woodii, P. Aerophilum and G. thermodenitrificans Deoxyribose-5-P Aldolases (DERAs)

PaDERA was obtained by commercial DNA synthesis using the published gene sequence as a template (NCBI GID: 1465578) and was supplied on a plasmid. The G. thermodenitrificans DERA was isolated as described above. E. coli and A. woodii were directly isolated from their respective genomic DNA with primers designed using the published genome sequences as templates. Using methods well known in the art in order to generate the final expression constructs; each of the target DERAs was PCR-amplified with 5′ NdeI and 3′ BamHI restriction sites and then independently subcloned using standard restriction enzyme-based cloning methods into the corresponding sites of the pET3a expression plasmid backbone, such that they were in frame with the plasmid-encoded T7 inducible promoter.

### Expression and Preparation of Lysates Containing DERAs for 1,3-Butanediol Production

Strains of E. coli BL21 Star (DE3) bearing either an empty pET3a vector or a cloned DERA from E. coli, G. thermodenitrificans, A. woodii and P. aerophilum were grown in 50 mL of commercial auto-induction medium (Formedium) containing 100 μg/ml carbenicillin, at 30° C. with shaking at 250 rpm. Following overnight growth, bacteria were lysed by bead-beating as described previously. The resulting lysates were clarified by centrifugation prior to activity assays. DERA activity for each lysate was determined in the retro aldol direction against deoxyribose-5-phosphate using a NADH linked assay for detection of the product acetaldehyde. The assay was carried out using 0.15 mM NADH, 5 mM 2-Deoxyribose 5-phosphate (Sigma: D3126) and 10 U/ml alcohol dehydrogenase (Sigma: A7011)

Lysates were diluted and reactions were run at the following volumetric and specific activities:

| | Volumetric | Specific |
|---|---|---|
| Cloned DERA | activity | Activity |
| Escherichia coli | 2 U/ml and | 0.09 U/mg and |
| | 20 U/ml | 0.9 U/mg |
| Geobacillus | 2 U/ml and | 0.09 U/mg and |
| thermodenitrificans | 20 U/ml | 0.9 U/mg |
| Acetobacterium | 2 U/ml and | 0.09 U/mg and |
| woodii | 20 U/ml | 0.9 U/mg |
| Pyrobaculum | 0.3 U/ml | 0.021 U/mg |
| aerophilum | | |

Acetaldehyde was supplied to the enzyme system (1 mL) via decarboxylation of pyruvate using yeast pyruvate decarboxylase (PDC) 0.5 U/ml (Sigma: P9474). Pyruvate was added at 5 and 30 mM. The reaction also contained the cloned DERA at either 2, 20 or 0.3 U/ml as appropriate and 0.033 mg/ml of purified GOX1615 (Example 9). The assays were carried out in 10 mM sodium phosphate buffer, pH 7 containing 0.1 mM thiamine pyrophosphate, 1 mM MgSO₄ and 1 mM DTT. Recycling of the cofactor NADPH for GOX1615 (0.15 mM) was provided by glucose dehydrogenase (GDH) from Pseudomonas sp. (Sigma: 19359) added at a final concentration of 0.1 mg/ml and 10 mM glucose. The reaction was incubated at 25° C., shaking at 250 rpm for an arbitrary 96 hr and was cooled on ice prior analysis. The concentration of reactants and products were monitored by HPLC (Phenomenex Rezex OA column organic acid H+300×7.8 mm).

A control comprised 5 and 30 mM pyruvate, 0.5 U/ml PDC, 0.15 mM NADPH, 0.1 mg/mL GDH, 10 mM glucose and 0.033 mg/ml GOX 1615 in buffer, pH 7.0 without addition of DERA lysate. This control confirmed that no 1,3-butanediol was produced via abiotic chemical condensation of acetaldehyde to 3-hydroxybutanal.

### Results

1,3-Butanediol Production (Escherichia coli DERA)

Pyruvate supplied at 5 mM

| | [Acetaldehyde] | [1,3 Butanediol] | [Ethanol] |
|---|---|---|---|
| DERA U/ml | mM | mM | mM |
| 2 | ND | 0.23 | 1.86 |
| 20 | ND | 0.31 | 3.67 |

Pyruvate supplied at 30 mM

| | [Acetaldehyde] | [1,3 Butanediol] | [Ethanol] |
|---|---|---|---|
| DERA mg/ml | mM | mM | mM |
| 2 | 23.80 | 0.54 | 5.68 |
| 20 | ND | 2.32 | 7.74 |

Control PDC and GOX1615 no DERA

| [Pyruvate] | [Acetaldehyde] | [1,3 Butanediol] | [Ethanol] |
|---|---|---|---|
| mM | mM | mM | mM |
| 5 | 3.56 | ND | 1.37 |
| 30 | 26.11 | ND | 5.62 |

1,3-Butanediol Production (Acetobacterium woodii DERA)

Pyruvate supplied at 5 mM

| | [Acetaldehyde] | [1,3 Butanediol] | [Ethanol] |
|---|---|---|---|
| DERA U/ml | mM | mM | mM |
| 2 | ND | 0.32 | 1.54 |
| 20 | ND | 0.37 | 2.69 |

Pyruvate supplied at 30 mM

| | [Acetaldehyde] | [1,3 Butanediol] | [Ethanol] |
|---|---|---|---|
| DERA mg/ml | mM | mM | mM |
| 2 | 23.4 | 0.95 | 5.31 |
| 20 | ND | 3.19 | 6.32 |

Control PDC and GOX1615 no DERA

| [Pyruvate] | [Acetaldehyde] | [1,3 Butanediol] | [Ethanol] |
|---|---|---|---|
| mM | mM | mM | mM |
| 5 | 3.56 | ND | 1.37 |
| 30 | 26.11 | ND | 5.62 |

1,3-Butanediol Production (Geobacillus thermodenitrificans DERA)

Pyruvate supplied at 5 mM

| | [Acetaldehyde] | [1,3 Butanediol] | [Ethanol] |
|---|---|---|---|
| DERA U/ml | mM | mM | mM |
| 2 | ND | 0.64 | 3.00 |
| 20 | ND | 0.24 | 7.45 |

Pyruvate supplied at 30 mM

| | [Acetaldehyde] | [1,3 Butanediol] | [Ethanol] |
|---|---|---|---|
| DERA mg/ml | mM | mM | mM |
| 2 | ND | 3.39 | 5.46 |
| 20 | ND | 2.19 | 14.55 |

Control PDC and GOX1615 no DERA

| [Pyruvate] | [Acetaldehyde] | [1,3 Butanediol] | [Ethanol] |
|---|---|---|---|
| mM | mM | mM | mM |
| 5 | 3.61 | ND | 0.82 |
| 30 | 26.83 | ND | 6.41 |

1,3-Butanediol Production (Pyrobaculum aerophilum DERA)

Pyruvate supplied at 5 mM

| | [Acetaldehyde] | [1,3 Butanediol] | [Ethanol] |
|---|---|---|---|
| DERA U/ml | mM | mM | mM |
| 0.3 U/ml | ND | 1.03 | 3.27 |

Pyruvate supplied at 30 mM

| | [Acetaldehyde] | [1,3 Butanediol] | [Ethanol] |
|---|---|---|---|
| DERA mg/ml | mM | mM | mM |
| 0.3 | ND | 4.36 | 3.25 |

Control PDC and GOX1615 no DERA

| [Pyruvate] | [Acetaldehyde] | [1,3 Butanediol] | [Ethanol] |
|---|---|---|---|
| mM | mM | mM | mM |
| 5 | 3.63 | ND | 1.98 |
| 30 | 27.6 | ND | 6.09 |

ND = none detected

1,3-butanediol was confirmed in all cases by LC/mass spectrometry. A representative mass spectrum is shown in FIG. 16.

### Example 14. Production of 1,3-Butanediol from Pyruvate Using a Cloned Full Pathway Operon

### Introduction

For exemplification of a complete 1,3-BDO biosynthetic pathway expressed as an operon, EcDERA, PDC1 and GOX1615 genes were assembled as a single polycistronic operon, under a lactose-inducible T7 promoter (in expression vector pET3a) and were actively expressed to produce 1,3-BDO in E. coli BL21 Star (DE3) cell lysate.

The aldehyde oxidoreductase (eutE) from Salmonella enterica subsp. enterica serovar Typhimurium strain LT2 (Uniprot ID: P41793, Genbank GID: 1253985) was initially used as an endogenous source of acetaldehyde substrate for EcDERA. In a later embodiment cloned PDC1 from Saccharomyces cerevisiae was used to replace eutE in this construct, as described below.

Construction of EcDERA and PDC with GOX1615 Expression Vectors

The PDC1 gene was PCR cloned from S. cerevisiae genomic DNA (SG ID S000004034 and Candy et al. 1991) with 24 bp of homology for 5′ UTR of GOX1615 in plasmid pDH384 (pET3a-EcDERA-EutE-GOX1615). The purified PCR product was then spliced into pDH384 using EMP PCR; such that PDC1 would replace the eutE coding sequence in the final construct and would also be cloned in-frame with the original ribosome-binding site; creating expression plasmid pDH527 (pET3a-EcDERA-PDC1-GOX1615).

### Expression and Preparation of Lysates Containing the Expressed Operon

Cell lysates from induced strains of E. coli BL21 Star (DE3) were prepared as described in Example 13.

The assays were carried out in 10 mM sodium phosphate buffer, pH 7 containing 0.1 mM thiamine pyrophosphate, 1 mM MgSO₄ and 1 mM DTT. Pyruvate was added to a final concentration of 5 mM and 30 mM. The lysate was diluted to contain units of expressed PDC, DERA and GOX 1615 as described below. The reaction was incubated at 25° C., shaking at 250 rpm for an arbitrary 96 hr and was cooled on ice prior to analysis by HPLC as described in Example 13.

The activity of the cloned DERAs was carried out using a NADH linked assay using 2-deoxyribose-5-phosphate as the substrate. The assay was carried out using 0.15 mM NADH, 5 mM 2-Deoxyribose 5-phosphate (Sigma: D3126) and 10 U/ml alcohol dehydrogenase (Sigma: A7011). The activity of PDC was carried out using a linked assay using 10 mM sodium pyruvate, 0.15 mM NADH and 10 U/ml alcohol dehydrogenase (Sigma: A7011). The activity of GOX 1615 was carried out using 10 mM butanal and 0.15 mM NADPH.

| | Volumetric | |
|---|---|---|
| Activity of each | activity in | |
| pathway enzyme | each reaction | Specific |
| in the reaction | as appropriate | Activity |
| DERA measured | 2 U/ml or | 0.22 U/mg or |
| against 2- | 20 U/ml | 2.2 U/mg |
| deoxyribose-5- | | |
| phosphate) | | |
| PDC | 0.28 U/ml or | 0.03 U/mg or |
| measured against | 2.8 U/ml | 0.3 U/mg |
| pyruvate | | |
| GOX1625 | 0.06 U/ml and | 0.007 U/mg and |
| measured against | 0.6 U/ml | 0.07 U/mg |
| butanal | | |

### Results

Pyruvate supplied at 5 mM

| | [Acetaldehyde] | [1,3 Butanediol] | [Ethanol] |
|---|---|---|---|
| DERA U/ml | mM | mM | mM |
| 2 | ND | 0.15 | 2.00 |
| 20 | ND | 0.45 | 3.63 |

Pyruvate supplied at 30 mM

| | [Acetaldehyde] | [1,3 Butanediol] | [Ethanol] |
|---|---|---|---|
| DERA U/ml | mM | mM | mM |
| 2 | ND | 0.82 | 5.21 |
| 20 | 3.24 | 4.01 | 7.67 |

Control PDC and GOX1615 no DERA)

| [Pyruvate] | [Acetaldehyde] | [1,3 Butanediol] | [Ethanol] |
|---|---|---|---|
| mM | mM | mM | mM |
| 5 | ND | ND | 4.05 |
| 30 | ND | ND | 7.94 |

ND = none detected

1,3-butanediol was confirmed in all cases by LC/mass spectrometry. A representative mass spectrum is shown in FIG. 16.

These data successfully demonstrate the ability for synthesis of 1,3-butanediol from a fully cloned novel, unnatural metabolic pathway, containing the key enzyme DERA for condensation of two molecules of acetaldehyde.

### REFERENCES FOR EXAMPLE 14

Candy J M, Duggleby R G, Mattick J S. Expression of active yeast pyruvate decarboxylase in Escherichia coli. J Gen Microbiol. 1991 December; 137(12):2811-5.

1. A non-naturally occurring microbial organism which includes a genetic modification in its genome which enhances production of 3-hydroxybutanal or a downstream product of 3-hydroxybutanal by the microbial organism from at least one endogenous central metabolic intermediate via a 3-hydroxybutanal synthetic pathway in which two molecules of acetaldehyde are condensed to form said 3-hydroxybutanal using an aldolase capable of accepting acetaldehyde as both the acceptor and donor in an aldol condensation.

2. (canceled)

3. A non-naturally occurring microbial organism as claimed in claim 1 wherein
the genetic modification:
(i) introduces a heterologous gene encoding an enzyme having an activity utilised in generation of acetaldehyde from one or more of the central metabolic intermediates;
(ii) up-regulates at least one endogenous enzyme having an activity utilised in generation of acetaldehyde from one or more of the central metabolic intermediates; and/or
(iii) down-regulates or inactivates an endogenous enzyme which utilises acetaldehyde as a substrate,
thereby increasing production or availability to the aldolase of the acetaldehyde, thereby increasing production of the 3-hydroxybutanal from the aldolase, and/or
wherein the genetic modification is either:
(i) the introduction of at least one heterologous gene encoding the aldolase, or
(ii) the up-regulation of at least one endogenous gene encoding the aldolase, optionally wherein the heterologous gene encodes the aldolase as a fusion protein encoding also one or more other enzymes involved in the 3-hydroxybutanal pathway.

4. A microbial organism as claimed in claim 1 wherein the genetic modification confers on the microorganism the capability to produce an increased amount of 3-hydroxybutanal or a downstream product of 3-hydroxybutanal, wherein the downstream product is obtained directly or indirectly from reduction, oxidation and\or acylation with Coenzyme A of 3-hydroxybutanal,
wherein the downstream product is:
(i) selected from 1,3-BDO; 2-hydroxisobutyrate, Crotyl alcohol, Crotonic acid, Butanol, Butyrate, 3-hydroxybutyrate, 3-hydroxybutylamine, Polyhydroxybutyrate, Acetone, Isopropanol, 2-methylsuccinic acid, and\or
(ii) is obtained via an intermediate selected from: 3-hydroxybutyryl CoA, 2-hydroxyisobutyryl CoA, Crotonyl CoA, Crotonaldehyde, Butyryl CoA, Butanal, Acetoacetyl CoA, and acetoacetate.

5. (canceled)

6. A microbial organism as claimed in claim 1 wherein the microorganism lacks the ability to produce the downstream product in the absence of said modification.

7-8. (canceled)

9. A microbial organism as claimed in claim 3 wherein said one or more other enzymes of the aldolase fusion protein provide an activity enhancing the provision of the aldolase substrate acetaldehyde or provide an activity converting the 3-hydroxybutanal to the downstream product.

10-11. (canceled)

12. A microbial organism as claimed in claim 3 wherein the aldolase is deoxyribose phosphate aldolase (DERA).

13. (canceled)

14. A microbial organism as claimed in claim 1 wherein the downstream product is 1,3-BDO and the genetic modification causes down-regulation or inactivation of an endogenous alcohol dehydrogenase with a preference for reduction of acetaldehyde to ethanol relative to reduction of 3-hydroxybutanal to 1,3-BDO.

15. A microbial organism as claimed in claim 1 wherein the central metabolic intermediate is selected from one or both of: acetyl CoA or pyruvate.

16-17. (canceled)

18. A microbial organism as claimed in claim 1 wherein the genetic modification comprises the introduction of a heterologous gene encoding one or more of the following 3-hydroxybutanal pathway enzymes:
(i) an enzyme having an activity utilised in generation of the central metabolic intermediates from feedstock;
(ii) an enzyme having an activity utilised in generation of acetaldehyde from the central metabolic intermediates;
(iii) an enzyme having an activity utilised in generation of the downstream product of 3-hydroxy butanal.

19. A microbial organism as claimed in claim 1 wherein acetyl CoA and pyruvate are generated in the organism by one or more of the following metabolic pathways encoded in the organism genome: the Wood-Ljungdahl pathway; the ribulose monophosphate (RuMP) pathway; the reverse TCA cycle; the serine cycle; glycolysis; the pentose phosphate pathway, the Calvin cycle, the 3-hydroxypropionate cycle; the dicarboxylate cycle/4-hydroxybutyrate cycle.

20. (canceled)

21. A microbial organism as claimed in claim 19 which is an acetogen,
which has the Wood-Ljungdahl pathway naturally encoded in its genome, wherein the genetic modification confers the ability to produce 3-hydroxybutanal or the downstream product thereof or the ability to produce an increased flux of 3-hydroxybutanal or the downstream product thereof from a feedstock selected from: syngas, CO₂, CO, and H₂, methanol, sugar or combinations thereof, optionally
wherein the genetic modification causes down-regulation or inactivation of an endogenous enzyme converting acetyl CoA to acetate, such as phosphotransacetylase or an endogenous acetate kinase.

22. A microbial organism as claimed in claim 19 which encodes in its genome one or more methyltransferase enzymes utilised in generation of the central metabolic intermediates from methanol feedstock, wherein said methyltransferase enzyme or enzymes are optionally heterologous to the microbial organism,
wherein the methyltransferase enzyme or enzymes are selected from the list consisting of:
methanol methyltransferase (MtaB); Corrinoid protein (MtaC);
Methyltetrahydrofolate: corrinoid protein methyltransferase (MtaA);
Methyltetrahydrofolate:corrinoid protein methyltransferase (AcsE); Corrinoid iron-sulfur protein (AcsD).

23.-28. (canceled)

29. A microbial organism as claimed in claim 1 wherein the genetic modification comprises the introduction of a heterologous gene or up-regulates at least one endogenous gene which encodes an enzyme activity which confers the ability to produce 3-hydroxybutanal or a downstream product thereof or the ability to produce an increased flux of 3-hydroxybutanal or a downstream product thereof from acetate, or the central metabolic intermediates acetyl CoA or pyruvate such that the 3-hydroxybutanal or a downstream product thereof accumulates and can be recovered or further converted enzymatically or chemically.

30. (canceled)

31. A microbial organism as claimed in claim 29 wherein the enzyme activity is used in the generation of acetyl CoA from acetate and is an acetyl CoA synthetase or a CoA transferase optionally selected from EC 6.2.1.1 or EC 2.8.3.8.

32. (canceled)

33. A microbial organism as claimed in claim 29 wherein the enzyme activity is a carboxylic acid reductase activity, optionally wherein the carboxylic acid reductase activity is selected from an enzyme of Table 1 or a variant of an enzyme of Table 1 capable of the reduction of acetate to acetaldehyde.

34.-35. (canceled)

36. A microbial organism as claimed in claim 29 wherein the enzyme activity is utilised in generation of acetaldehyde from acetyl CoA, optionally
wherein the enzyme activity is an aldehyde dehydrogenase, optionally an acetaldehyde dehydrogenase.

37.-41. (canceled)

42. A microbial organism as claimed in claim 29 wherein the enzyme activity is utilised in generation of acetaldehyde from pyruvate, optionally
wherein the enzyme activity is a pyruvate decarboxylase.

43.-46. (canceled)

47. A microbial organism as claimed in claim 42 wherein the genetic modification causes down-regulation or inactivation of an endogenous enzyme capable of converting pyruvate to lactate or other products.

48. A microbial organism as claimed in claim 29 wherein the endogenous enzyme capable of converting pyruvate to lactate or other products is an LDH or malate dehydrogenase or pyruvate formate lyase, optionally selected from 1.1.1.27 or 1.1.1.37 or 2.3.1.54.

49. A microbial organism as claimed in claim 47 wherein the genetic modification comprises the introduction of a heterologous gene or up-regulates at least one endogenous gene which encodes an enzyme activity which confers the ability to convert 3-hydroxybutanal to 1,3-BDO, optionally
wherein the enzyme has alcohol dehydrogenase activity or has aldehyde reductase activity optionally classified as EC 1.1.1.-.

50.-52. (canceled)

53. A process method for producing 3-hydroxybutanal or a downstream product thereof, which process comprises culturing a microbial organism as claimed in claim 1 for a sufficient period of time to produce said 3-hydroxybutanal or the downstream product thereof.

54.-59. (canceled)

60. A process for producing a microbial organism according to claim 1, which comprises introducing said genetic modification into a parent strain.

61.-65. (canceled)


SEQUENCE LISTING (36 sequences)

SEQ ID NO: 1 - DNA, 28 bp, Artificial sequence
  Synthetic primer EryFor of PCT/EP2015/072552
ggggatccaatgatacaccaatcagtgc

SEQ ID NO: 2 - DNA, 27 bp, Artificial sequence
  Synthetic primer EryXbaRev of PCT/EP2015/072552
ggtctagattgaacccgtctccttacg

SEQ ID NO: 3 - DNA, 27 bp, Artificial sequence
  Synthetic primer 01For(uplacdehyEco) of PCT/EP2015/072552
gggaattcatgtcgatcatattgaagg

SEQ ID NO: 4 - DNA, 23 bp, Artificial sequence
  Synthetic primer 01Rev(uplacdehyBam) of PCT/EP2015/072552
aaggatcccttaaacgaccatcc

SEQ ID NO: 5 - DNA, 25 bp, Artificial sequence
  Synthetic primer 02For(dwnlacdehyXba) of PCT/EP2015/072552
cctctagagtgagatgatggataac

SEQ ID NO: 6 - DNA, 37 bp, Artificial sequence
  Synthetic primer 02Rev(dwnlacdehyPstHind) of PCT/EP2015/072552
gcaagcttctgcagtcatccaaatgtgcttaacaacc

SEQ ID NO: 7 - DNA, 24 bp, Artificial sequence
  Synthetic primer 16For of PCT/EP2015/072552
aatctagattagtcggtgcctgtg

SEQ ID NO: 8 - DNA, 23 bp, Artificial sequence
  Synthetic primer 16Rev of PCT/EP2015/072552
ggaagctttctttacgttctacg

SEQ ID NO: 9 - DNA, 26 bp, Artificial sequence
  Synthetic primer 13b(LDHantiFor50) of PCT/EP2015/072552
aaactagtggcgattccaacggcgat

SEQ ID NO: 10 - DNA, 32 bp, Artificial sequence
  Synthetic primer 13(LDHantiRev) of PCT/EP2015/072552
aactcgagctacatctgacagacttttttcgg

SEQ ID NO: 11 - DNA, 20 bp, Artificial sequence
  Synthetic primer pAMbeta1For of PCT/EP2015/072552
cgatttccgattgattgctt

SEQ ID NO: 12 - DNA, 20 bp, Artificial sequence
  Synthetic primer pAMbeta1Rev of PCT/EP2015/072552
aatcccaaatgagccaacag

SEQ ID NO: 13 - DNA, 26 bp, Artificial sequence
  Synthetic primer Pr1_cisFor of PCT/EP2015/072552
ggactagttgttatttggcgatcagc

SEQ ID NO: 14 - DNA, 24 bp, Artificial sequence
  Synthetic primer Pr4_iorRev of PCT/EP2015/072552
ggctgcagcgcacccatacaaagc

SEQ ID NO: 15 - DNA, 41 bp, Artificial sequence
  Synthetic primer Pr5_PTAfrag1Rev of PCT/EP2015/072552
aacatcaacatgcggccgcacttaccaaattatctgcgtcg

SEQ ID NO: 16 - DNA, 41 bp, Artificial sequence
  Synthetic primer Pr6_PTAfrag3For of PCT/EP2015/072552
aatttggtaagtgcggccgcatgttgatgttattctcatgc

SEQ ID NO: 17 - DNA, 109 bp, Artificial sequence
  Synthetic primer Pr55 of PCT/EP2015/072552
gagtcgacgcagtatcttaaaattttgtataataggaattgaagttaaattagatgctaaaaatttgtaattaagaaggagtgattacatgttacgtcctgtagaaacc

SEQ ID NO: 18 - DNA, 24 bp, Artificial sequence
  Synthetic primer Pr54 of PCT/EP2015/072552
ttgcatgctcattgtttgcctccc

SEQ ID NO: 19 - DNA, 18 bp, Artificial sequence
  Synthetic primer APB57 of PCT/EP2015/072552
agctttcgagcgcggaac

SEQ ID NO: 20 - DNA, 23 bp, Artificial sequence
  Synthetic primer APB58 of PCT/EP2015/072552
gagttccatgtggtctaccatac

SEQ ID NO: 21 - DNA, 35 bp, Artificial sequence
  Synthetic primer APB59 of PCT/EP2015/072552
catggaggttaaggctgagttgacgatacactgtc

SEQ ID NO: 22 - DNA, 32 bp, Artificial sequence
  Synthetic primer APB60 of PCT/EP2015/072552
gtcaactcagccttaacctccatgacgaccag

SEQ ID NO: 23 - DNA, 48 bp, Artificial sequence
  Synthetic primer APB61 of PCT/EP2015/072552
gacgagcaaggcaagaccgggatccgacagtaaccgtaggtaccttcg

SEQ ID NO: 24 - DNA, 24 bp, Artificial sequence
  Synthetic primer APB62 of PCT/EP2015/072552
ccagtgatctctttatcgacctcc

SEQ ID NO: 25 - DNA, 34 bp, Artificial sequence
  Synthetic primer APB63 of PCT/EP2015/072552
ggtgtgcatgtgcaaggacacgcaccttttctag

SEQ ID NO: 26 - DNA, 31 bp, Artificial sequence
  Synthetic primer APB64 of PCT/EP2015/072552
tgcgtgtccttgcacatgcacaccgatgagg

SEQ ID NO: 27 - DNA, 46 bp, Artificial sequence
  Synthetic primer APB65 of PCT/EP2015/072552
gacgagcaaggcaagaccgggatccgcttcaacccaagcttgtagc

SEQ ID NO: 28 - DNA, 24 bp, Artificial sequence
  Synthetic primer Pr89 of PCT/EP2015/072552
gccatatggcatccgacaccatcc

SEQ ID NO: 29 - DNA, 21 bp, Artificial sequence
  Synthetic primer Pr90 of PCT/EP2015/072552
ccggatcctcagtcccgtgcc

SEQ ID NO: 30 - DNA, 25 bp, Artificial sequence
  Synthetic primer Pr91 of PCT/EP2015/072552
gccatatgtcagttttcgtttcagg

SEQ ID NO: 31 - DNA, 22 bp, Artificial sequence
  Synthetic primer Pr92 of PCT/EP2015/072552
cggatccttatattctgccctc

SEQ ID NO: 32 - DNA, 27 bp, Artificial sequence
  Synthetic primer APB142F of PCT/EP2015/072552
aatcaacaggatattgaacaggtggtg

SEQ ID NO: 33 - DNA, 20 bp, Artificial sequence
  Synthetic primer APB143R of PCT/EP2015/072552
gtagctgctggcgctcttac

SEQ ID NO: 34 - DNA, 28 bp, Artificial sequence
  Synthetic primer APB106F of PCT/EP2015/072552
atgacggtgaatattgctaaaatgatcg

SEQ ID NO: 35 - DNA, 24 bp, Artificial sequence
  Synthetic primer APB107R of PCT/EP2015/072552
ttaatagtcagcgccgccggtttg

SEQ ID NO: 36 - DNA, 672 bp, Geobacillus thermodenitrificans
atgacggtgaatattgctaaaatgatcgatcatacgttgcttaagccagaagcgacggaagagcaaatcattcaactatgcgacgaagcaaagcaacacggcttcgcctcggtgtgcgtcaacccagcgtgggtgaaaacagcggcacgcgagctttccgacactgatgtccgcgtctgcacggtcatcggctttccgcttggggcgacgacgccggaaacaaaggcgtttgaaacgaacaacgctatcgaaaacggcgcccgcgaagtcgatatggtaatcaacatcggcgcgttaaaaagtggtaacgatgaactcgttgagcgcgacattcgtgcggttgttgaggcggcgtccgggaaagcgcttgtgaaagtgatcatcgaaacggccttgttgactgatgaggaaaaagtgcgcgcctgccaattggcggtgaaagcgggcgccgattacgtaaaaacgtcgaccggattctcaggcggcggagcgacggtcgaagacgtggcgctgatgcgccggacagttggcgataaagcaggtgtcaaagcctcaggaggcgtccgcgaccgaaaaacagccgaagcgatgattgaagctggggccacgcgcattgggacgagctccggggtggcgatcgtcagcggccaaaccggcggcgctgactattaa